(19) United States
(12) Patent Application Publication
(10) Pub. No.: US 2008/0311589 A1
(43) Pub. Date: Dec. 18, 2008

(54) METHOD OF HIGH-THROUGHPUT SCREENING OF MOLECULES AND COMPOUNDS FOR THEIR EFFECTS ON BIOLOGICAL AND CHEMICAL PROCESSES

(76) Inventors: Brent R. Stockwell, Boston, MA (US); Stuart L. Schreiber, Boston, MA (US); Timothy J. Mitchison, Brookline, MA (US); Tarun M. Kapoor, Cambridge, MA (US); Thomas Mayer, Brookline, MA (US); Stephen J. Haggarty, Somerville, MA (US)

Correspondence Address: CHOATE, HALL & STEWART LLP, TWO INTERNATIONAL PLACE, BOSTON, MA, 02110, US

(21) Appl. No.: 12/107,042

(22) Filed: Apr. 21, 2008

Related U.S. Application Data
(63) Continuation of application No. 09/361,576, filed on Jul. 27, 1999, now abandoned.

(51) Int. Cl. G01N 33/53

(52) U.S. Cl. 435/7.1

(57) ABSTRACT

The present invention provides a system for high-throughput analysis of chemical compounds. Assays are performed in a high density platform, and compounds having pre-determined desirable effects are identified. Preferably, the compounds have biological effects, more preferably, the assays and detection are performed on whole cells.

This application claims priority under 35 U.S.C. 119(e) to the provisional application U.S. Ser. No. 60/094,305 entitled “Method of High-throughput Screening of Small Molecules for Their Effects on Cellular Activity” filed Jul. 27, 1998 and hereby incorporated in its entirety by reference.

This application also claims priority under 35 U.S.C. 119(e) to the provisional application U.S. Ser. No. 60/131,765 entitled “Novel Cell Cycle Inhibitors that Affect the Cytoskeleton and Mitosis” filed Apr. 30, 1999, which is also hereby incorporated in its entirety by reference.

This application also claims priority under 35 U.S.C. 119(e) to the provisional application U.S. Ser. No. 60/137,039 filed Jun. 1, 1999 entitled “Metal Binding Agents”, which is also incorporated herein in its entirety by reference.

### BACKGROUND OF THE INVENTION

A major goal of biomedical research is the identification of molecules and compounds that can modulate specific biological processes. Currently, molecules are being designed to interact with the active sites of proteins whose structures have been elucidated (Ferenczy, G. Acta. Pharm. Hung. 68:21-31. 1998; Blundell, T. L. Nature 384:23-26. 1996). In addition, methods are being developed to identify protein-small molecule interactions (Borchardt et al. Chem. Biol. 4:961-968. 1997).

The development of high-throughput assays to screen large collections of molecules and identify those that can interact with a specific protein target has been a major goal of academic and industrial research laboratories. However, the majority of assays employed in these screens either detect specific protein-ligand interactions using recombinant proteins or study the effects of small molecules on the growth of cells, without concern for the specific signaling pathways involved (Borchardt et al. Chem. Biol. 4:961-968. 1997; Huang & Schreiber. Proc Natl Acad Sci, USA 94:13396-13401. 1997; Combs et al. J. Am. Chem. Soc. 118:287-288. 1996).

Detection of cell growth and proliferation has traditionally been performed by tedious and labor-intensive methods such as direct cell counting, determination of the mitotic index or clonogenic assays (see Product Specifications for Cell Proliferation ELISA, BrdU chemiluminescence; Boehringer Mannheim Corporation, Roche Molecular Biochemicals, Basel, Switzerland, Cat.# 1-669-915 and references therein). These methods are not practical for high-throughput screens, where a large number of samples are assayed. Other cell growth assays employ tetrazolium dyes, such as MTT, XTT, or WST-1, whose metabolism can be used as an indicator of cellular activity. This approach can be applied to standard 96-well multiwell formats, but its low sensitivity restricts its usefulness in higher density arrays where the number of metabolically active cells may be very low in comparison to the sample size.

Appropriately designed cell-based assays have the potential to identify small molecules that affect specific signaling pathways in vivo. In particular, a recent report has described a method for identifying molecules that disrupt a specific biological process, activation of endothelial cells by interleukin (IL)-1β (Rice et al. Anal. Biochem. 241:254-259. 1996). IL-1β activation results in E-selectin production, so that activated endothelial cells have E-selectin molecules on their surfaces. The Rice et al. assay screened for the absence of E-selectin on cell surfaces after cells had been exposed to small molecules. The assay was performed in 96-well plates containing approximately 20,000 cells per well and detected E-selectin with a monoclonal antibody that was subsequently detected with a secondary antibody coupled to horse radish peroxidase (HRP) and reacted with o-phenylenediamine (OPD). The authors screened approximately 113,000 compounds over a period of three months.

Although the Rice et al. assay was successful in the one context in which it was employed, there is no indication that the approach could be generalized to higher density formats. Moreover, the strategy is limited to the detection of cell surface markers and cannot analyze processes occurring inside cells.

Other biological assays that have been developed in high-throughput formats use immobilized cell lysates in the bottom of assay plates as the source of antigens for detection in an enzyme-linked immunosorbent assay (ELISA). For example, this method has been used to screen fractionated cell extracts for phosphatase activity through detection of reduced reactivity of an antibody (MPM-2) directed towards a phosphospecific epitope generated in mitotic cell extracts of Xenopus laevis (Che et al. FEBS Lett. 424:225-33. 1998).

There remains a need for the development of high throughput formats for screening compounds that can participate in or disrupt biological processes. There is a particular need for the development of high throughput systems that allow the analysis of events occurring inside cells.

### SUMMARY OF THE INVENTION

The present invention provides a system for the high throughput screening of chemical compounds. The system is particularly applicable to analysis of compounds that affect biological processes. In preferred embodiments, the invention detects events that occur inside cells. For example, the inventive system may be applied the detection of compounds that alter the intracellular concentration of a target biological compound. Alternatively or additionally, the inventive system may be employed to identify compounds that suppress or enhance a specific biological phenotype. In preferred embodiments, the compounds analyzed comprise compounds synthesized by combinatorial chemistry.

In one preferred embodiment, the inventive system is used to determine functional profiles for chemical compounds, assaying their activities in multiple different contexts. To give but one example, a particular compound's effects may be determined in various cell types of different genetic backgrounds, tissue origins, and/or stages of development.

One of the advantages of the present invention is that it allows the rapid analysis of large numbers of chemical compounds. The systems described herein are miniaturizable, allowing reduced sample size and therefore reduced reagent cost. Furthermore, large numbers of reactions can be performed simultaneously. The inventive assays can be performed with high stringency to facilitate high throughput screening of large libraries and to increase the probability of “hits”. The assays and detection systems can also be highly specific to ensure that any identified hits are relevant to the biological or chemical reaction of interest.

In certain embodiments, the present invention utilizes an assay format containing a plurality of reaction vessels arranged with sufficient density such that individual vessels are separated from one another by no more than about 5 millimeters. Preferably, the vessels are separated by no more than about 2 millimeters. More preferably, the vessels are separated by no more than about 1 millimeter. Most preferably, the vessels are separated by no more than about 0.25 millimeters.

Preferably, the present invention is employed to screen chemical compounds for their effects on biological systems. Preferably, the biological system includes at least one cell. More preferably, the cell is a eukaryotic cell. Even more preferably the cell is a mammalian cell. Most preferably, the cell is a human cell. In preferred embodiments, approximately 8000 mammalian cells are assayed in reaction; more preferably, fewer cells, such as 2000, 500, 100, or fewer, are employed.

In one preferred embodiment, the present invention screens chemical compounds for their effects on chemical and/or biological systems by detecting the present or amount of a component present or produced by the system, which component acts as a marker for the chemical or biological process of interest. Preferably, the component is detected by means of its interaction with a binding partner ligand. Preferably, the binding is specific. In certain preferred embodiments, the binding partner ligand is an antibody.

Interaction of a ligand and component is preferably detected through analysis of a detectable entity association with the ligand. In certain preferred embodiments, the detectable entity comprises a luminescent moiety. For example, the ligand may include a peroxidase that is capable of generating a chemiluminescent compound which can be detected.

In certain preferred embodiments, the present invention provides for a system of identifying compounds capable of affecting a biological or chemical process wherein the system comprises a high density array of reaction vessels containing at least 100 reaction vessels and a collection of compounds for screening. Preferably, the array of reaction vessels contains at least 300 reaction vessel and each vessel preferably has a volume less than or equal to approximately 50 microliters. More preferably, the array of reaction vessels contains at least 1000 reaction vessels and/or each vessel has a volume less than or equal to approximately 2 microliters. Even more preferably, the array of reaction vessels contains at least 5000 reaction vessels and/or each vessel has a volume less than or equal to approximately 250 nanoliters.

In another preferred embodiment, the present invention provides a system for identifying compounds capable of affecting a biological or chemical process comprising a high density array of reaction vessels containing at least 100 reaction vessels and an assay solution containing at least one reagent for detecting levels of component in a biological or a chemical process or resulting from a biological or a chemical process. Preferably, the array of reaction vessels contains at least 300 reaction vessels, and/or each vessel has a volume less than or equal to approximately 50 microliters, and/or the assay solution includes a component that is detected using chemiluminesce. More preferably, the array of reaction vessels contains at least 1000 reaction vessels, each vessel has a volume less than or equal to approximately 2 microliters, and/or the detected chemiluminescent compound is produced by a peroxidase. Most preferably, the array of reaction vessels contains at least 5000 reaction vessels, each vessel has a volume less than or equal to approximately 250 nanoliters, and/or the peroxidase is horseradish peroxidase.

The present invention also provides a method of stimulating expression of TGFβ-responsive genes by providing a system including one or more genes under the control of one or more TGFβ-responsive elements and contacting the system with a compound having a structure as set forth in FIG. 16 or FIG. 17.

The present invention also provides for a method of altering metal concentration in a system by providing a system in which metal concentration is to be adjusted, and contacting the system with a compound having a structure as set forth in FIG. 16 or FIG. 17.

The present invention further provides compounds and compositions that are useful as microtubule stabilizers and/or as specific effectors of the cytoskeleton, and well as methods for using such compounds and compositions.

### DESCRIPTION OF THE DRAWINGS

FIG. 1. An immunodetection assay for DNA synthesis in high density arrays of mammalian cells. (a) Cartoon depiction of an anti-BrdU cytoblot. The thymine analog 5-bromodeoxyuridine (BrdU) is incorporated into the DNA of cells that are actively replicating their DNA. The cells are in the well, and BrdU is detected with a two step antibody binding procedure. The second antibody is conjugated to the enzyme horseradish peroxidase. In the presence of the chemiluminescent substrate luminol as well as hydrogen peroxide, light of wavelength 428 nm is generated. The light emission can be detected by exposing the plate to film. (b) A cytoblot can detect TGF-β's ability to prevent BrdU incorporation in mink lung epithelial cells. 2000 Mv1Lu mink lung epithelial cells were seeded in each well of a white, opaque 384 well plate. The cells were seeded in the indicated concentrations of TGF-β in 50 μL of DMEM with 1% fetal bovine serum (FBS), 100 units/mL penicillin G sodium, 100 μg/mL streptomycin sulfate and 100 μM each of the amino acids alanine, aspartic acid, glutamine, glycine, asparagine and proline (referred to throughout as 1% mink medium) and allowed to incubate at 37° C. with 5% CO₂. After 16 hours, 5.5 μL of 100 μM BrdU in 1% mink medium was added to each well, for a final concentration of 10 μM BrdU. The cells were incubated at 37° C. with 5% CO₂ for an additional 16 hours and then an anti-BrdU cytoblot protocol was performed (see protocol). Wells shown are magnified 4×.

FIG. 2. A cytoblot can detect the ability of numerous antiproliferative agents to inhibit BrdU incorporation. 2000 Mv1Lu mink lung epithelial cells were seeded in each well of a white, opaque 384 well plate. The cells were seeded in 40 μL of 1% mink media and immediately 40 μL of 2× stocks of the reagents shown was added to each well and the cells were allowed to incubate at 37° C. with 5% CO₂. After 24 hours, 9 μL of 100 μM BrdU in 1% mink medium was added to each well, for a final concentration of 10 μM BrdU. The cells were incubated at 37° C. with 5% CO₂ for an additional 16 hours and then an anti-BrdU cytoblot protocol was performed (see protocol). Wells shown are magnified 2.5×.

FIG. 3. BrdU incorporation can be efficiently detected with a cytoblot in 1536 well plates in 2 μL droplets. 500 Mv1Lu mink lung epithelial cells were seeded in each well of a white, opaque 1536 well plate (Corning/Costar, Corning, N.Y.). The cells were seeded with or without 400 μM TGF-β in 2 μL of 1% mink medium and allowed to incubate at 37° C. with 5% CO₂. After 24 hours, 0.5 μL of 50 μM BrdU in 1% mink medium was added to the indicated wells, for a final concentration of 10 μM BrdU. The plate were incubated at 37° C. with 5% CO₂ for an additional 12 hours and then an anti-BrdU cytoblot protocol was performed (see protocols). Wells are magnified as indicated.

FIG. 4. BrdU incorporation is detected in 6144 well plates with a cytoblot. (a) The actual size of the wells of a 6144 well plate (nanowells; You et al. Chem. Biol. 4:969-975. 1997) is shown. (b) 25× magnification of the blot shown in (a). The indicated number of cells (Mv1Lu) were seeded in 250 mL volumes in duplicate in 1% mink medium with or without 10 μM BrdU or 400 pM TGF-β, as indicated. The cells were cultured for 24 hours at 37° C. with 5% CO₂ and then a 6144 well cytoblot was performed (see protocols). (c) Two separate nanowell plates (You et al. Chem. Biol. 4:969-975. 1997) were seeded with 20,000 Mv1Lu cells/mL in 1% mink medium with or without 500 pM TGF-13. After 26 hours at 37° C. with 5% CO₂ BrdU was added to a final concentration of 10 μM. The cells were incubated for an additional 18 hours at 37° C. with 5% CO₂ and then a 6144 well cytoblot was performed (see protocols).

FIG. 5. An immunodetection assay for the accumulation of hyperacetylated histone H4 in high density arrays of mammalian cells. A549 human lung carcinoma cells were seeded at a density of 4000 cells in 40 μL of DMEM+ in each well of an opaque 384 well plate and incubated overnight at 37° C. with 5% CO₂. Cells were either untreated, washed once and treated with 0.5% serum, 80 pM TGF-β, 300 nM trichostatin A, 100 nM trapoxin or 250 nM nocodazole and incubated for 24 hours at 37° C. with 5% CO₂ in a final volume of 50 μL. A cytoblot was performed (see Protocol) and the presence of the hyperacetylated form of histone H4 was detected using a two step antibody binding procedure using an anti-acetylated H4 antibody and a secondary antibody conjugated to the enzyme horseradish peroxidase. Wells are shown magnified 25×.

FIG. 6. (a) An immunodetection assay for the accumulation of phosphonucleolin as a marker of mitosis in high density arrays of mammalian cells. Varying densities of adherent HeLa cells were seeded in 40 μL of Dulbecco's modified Eagle's medium (DMEM) supplemented with 10% fetal bovine serum (FBS), 100 units/mL penicillin G sodium, 100 μg/mL streptomycin sulfate and 2 mM L-glutamine (referred to as DMEM+) in each well of an opaque white 384 well plate and incubated overnight at 37° C. with 5% CO₂. Either an equivalent amount of DMSO (0.08%) or 500 nM (final concentration) nocodazole in DMSO was added to each well for a final volume of 50 μL and cells incubated for an additional 24 hour period at 37° C. with 5% CO₂. A cytoblot was performed (see Protocol) and the presence of the phosphorylated form of nucleolin was detected using a two step antibody binding procedure using TG-3 and a secondary antibody conjugated to the enzyme horseradish peroxidase. Wells are shown magnified 4×. Similar results were obtained using other colchicine and other cell lines (primary and transformed; data not shown). (b) An immunodetection assay for the accumulation of phosphorylated histone H3 as a marker of mitosis in high density arrays of mammalian cells. A549 human lung carcinoma cells were seeded at a density of 4000 cells in 40 μL of DMEM+ in each well of an opaque 384 well plate and incubated overnight at 37° C. with 5% CO₂. Cells were either untreated or treated with nocodazole at a final concentration of 100 nM and incubated for 16 hours at 37° C. with 5% CO₂ in a final volume of 50 μL. A cytoblot was performed (see Protocol) and the presence of the phosphorylated form of histone H3 was detected using a two step antibody binding procedure using anti-phospho histone H3 mitosis marker and a secondary antibody conjugated to the enzyme horseradish peroxidase. Wells are shown magnified 25×. (c) The accumulation of phosphonucleolin as a result of nocodazole treatment can be efficiently detected with cytoblots in 1536 well plates. A549 human lung carcinoma cells at a density of 300,000 cells/mL were overlaid on 2 cm square portion of a 1536 well plate (You et al. Chem. Biol. 4:969-975. 1997.) to an approximate depth of 5 mm in DMEM+ and final density of approximately 1000 cells and incubated overnight at 37° C. with 5% CO₂. Either an equivalent amount of DMSO (0.08%) or 250 nM (final concentration) nocodazole in DMSO was added to each plate and cells incubated for an additional 24 hour period at 37° C. with 5% CO₂. A cytoblot was performed (see Protocol). Wells are shown magnified 4×. (d) Cytoblot assays for the accumulation of hyperacetylated histone H4, phosphonucleolin and phosphorylated histone H3. (e) 4000 A549 human lung carcinoma cells were seeded in 40 μL in a white 384 well plate, allowed to attach overnight and then either untreated (NT), or washed once and treated with 0.5% serum, 80 pM TGF-β, 300 nM trichostatin A (TSA), 100 nM trapoxin (trap) or 250 nM nocodazole (ncdz) and incubated for 24 hours at 37° C. with 5% CO₂ in a final volume of 50 μL. A cytoblot was performed and the presence of the hyperacetylated form of histone H4 was detected using anti-acetylated H4 antibody and a secondary antibody conjugated to HRP. (f) Human HeLaS3 cells were seeded in 40 μL in a white 384 well plate, allowed to attach overnight and either untreated (NT) or treated with trapoxin at a final concentration of 100 nM for the times indicated and incubated at 37° C. with 5% CO₂ in a final volume of 50 μL. A cytoblot was performed as in (a). (g) A549 cells were seeded in 40 μL in a white 384 well plate, allowed to attach overnight and either untreated (NT) or treated with nocodazole (ncdz) at a final concentration of 250 nM for the times indicated and incubated at 37° C. with 5% CO₂ in a final volume of 50 μL. A cytoblot was performed and the presence of the phosphorylated form of histone H3 detected using anti-phospho histone H3 mitosis marker and a secondary antibody conjugated to HRP. (h) HeLaS3 cells were seeded in 40 μL in a white 384 well plate, allowed to attach overnight and either untreated (NT) or treated with nocodazole (ncdz) at a final concentration of 500 nM for the times indicated and incubated at 37° C. with 5% CO₂ in a final volume of 50 μL. A cytoblot was performed and the presence of the phosphorylated form of nucleolin detected using the TG-3 antibody and a secondary antibody conjugated to HRP. (i) A sample of wells from a 6144-well plate were collectively incubated in 1 mL of A549 cells (500 cells per well) and were either untreated (NT) or treated with nocodazole (ncdz) at a final concentration of 500 nM and incubated for 24 hours at 37° C. with 5% CO₂ in a final volume of 50 μL. A cytoblot was performed. A 4 mm scale bar is shown for (a)-(d) and a 1 mm scale bar is shown for (e).

FIG. 7. FK506 suppresses the antiproliferative effect of rapamycin but not trapoxin. 2000 6F mink lung epithelial cells (6F cells, a stable cell line in which the small molecule FK1012 activates TGF-β signaling (Stockwell & Schreiber. Curr. Biol. 8:761-770. 1998), are more responsive to the growth inhibitory effects of rapamycin than the parental Mv1Lu cell line) were seeded in each well of a white, opaque 384 well plate. The cells were seeded in the indicated concentrations of rapamycin or trapoxin in 40 μL of 1% mink medium and immediately 40 μL of 2× stocks of the indicated concentrations of FK506 was added to each well and the cells were allowed to incubate at 37° C. with 5% CO₂. After 24 hours, 9 μL of 100 μM BrdU in 1% mink medium was added to each well, for a final concentration of 10 μM BrdU. The cells were incubated at 37° C. with 5% CO₂ for an additional 16 hours and then an anti-BrdU cytoblot protocol was performed (see protocol). Wells shown are magnified 2×.

FIG. 8. BrdU incorporation can be efficiently detected with cytoblots in high density plates using multiple cell lines. (a) 500 Mv1Lu mink lung epithelial cells were seeded in 2 μL in each well of a white, opaque 1536-well plate (Corning/Costar). The cells were seeded with or without 400 pM TGF-β in 2 μL of 1% mink medium and allowed to incubate at 37° C. with 5% CO₂. After 24 hours, 0.5 μL of 50 μM BrdU in 1% mink medium was added to the indicated wells, yielding a final concentration of 10 μM BrdU. The plate was incubated at 37° C. with 5% CO₂ for an additional 12 hours and then an anti-BrdU cytoblot protocol was performed. A 1.5 mm scale bar is shown. (b) Mv1Lu cells were seeded on plasma-cleaned 6144-well polydimethylsiloxane (PDMS) plates (Randy King, unpublished results) at a density of 100,000 cells/mL in 1% mink medium. After 6.5 hours the cells had attached and new medium with or without 500 pM TGF-β1 was added to the plates and the excess removed, leaving approximately 250 mL per well. The cells were incubated for 18.5 hours at 37° C. with 5% CO₂, then the medium was washed out and new 1% mink medium with 10 μM BrdU was added. After 90 minutes an anti-BrdU cytoblot was performed. A 1 mm scale bar is shown. (c) The indicated number of mouse embryonic stem cells were seeded in 90 μL of ES medium (DMEM, 15% characterized FBS (Hyclone), 0.1 mM β-mercaptoethanol (Sigma), 0.1 mM non-essential amino acids (GibcoBRL), 100 units/mL penicillin G sodium, 100 μg/mL streptomycin sulfate, 2 mM glutamine (GibcoBRL), 250 U/mL leukemia inhibitory factor (ESGRO, GibcoBRL)) on a 384 well plate that had been precoated with nothing (NT), poly-L-lysine (lysine) or 0.1% gelatin (gelatin). The cells were incubated for 24 hours at 37° C. with 5% CO₂ and then BrdU was added to a final concentration of 10 μM. After 12 hours an anti-BrdU cytoblot was performed. (d) 2500 MEFs (p53^{-/-} or p21^{Kip1-/-}) were seeded in MEF medium in 384 well white plates and cultured overnight at 37_C with 5% CO₂. Marine sponge extracts (from Professor Phillip Crews and Miranda Sanders) were pin-transferred (1.19 mm 96 pin array, V&P Scientific) in duplicate rows from 10 mg/mL dimethylsulfoxide (DMSO) stock solutions into 40 μL MEF medium for both cell lines. After 24 hours BrdU was added from a 10 mM PBS stock to a final concentration of 10 μM and the cells were cultured for an additional 8.5 hours. A BrdU cytoblot was performed on each plate. The film images of the results were scanned into Photoshop 5.0 (Adobe) and converted to inverse white/red and white/green color scales and merged, with one layer 50% transparent.

FIG. 9. Genetic-like screens using small molecules (a) Cartoon depiction of the ability of excess FK506 to suppress the anti-proliferative effect of rapamycin. Excess FK506 binds all available FKBP and thereby prevents rapamycin from binding FKBP. Rapamycin can not bind FRAP, and therefore does not inhibit proliferation, in the absence of FKBP. (b) FK506 suppresses the anti-proliferative activity of rapamycin, but not trapoxin. 2000 6F mink lung epithelial cells (6F cells, a stable cell line in which the small molecule FK1012 activates TGF-β signaling, are more responsive to the growth inhibitory effects of rapamycin than the parental Mv1Lu cell line (BRS and SLS, unpublished results)) were seeded in each well of a white, opaque 384-well plate. The cells were seeded in the indicated concentrations of rapamycin or trapoxin in 40 μL of 1% mink medium and immediately 40 μL of 2× stocks of the indicated concentrations of FK506 was added to each well and then the cells were allowed to incubate at 37° C. with 5% CO₂. After 24 hours, 9 μL of 100 μM BrdU in 1% mink medium was added to each well, for a final concentration of 10 μM BrdU. The cells were incubated at 37° C. with 5% CO₂ for an additional 16 hours and then an anti-BrdU cytoblot protocol was performed. A 4 mm scale bar is shown. (c) Identification of crude organic marine sponge extracts that are capable of suppressing rapamycin's anti-proliferative effect. 2000 6F mink lung epithelial cells were seeded in 50 μL of 1% mink media containing 20 nM rapamycin in each well of a white 384 well plate. 192 marine natural product extracts (10 mg/mL stock solution in DMSO) were assayed in duplicate rows by transferring approximately 50 nL to each assay well using a 96 pin array (V&P Scientific, cat. # VP409). After 49 hours, 10 μL of 6×BrdU was added to each well, yielding a final concentration of 10 μM BrdU. 13 hours later an anti-BrdU cytoblot was performed. A 4 mm scale bar is shown.

FIG. 10. The ability of anti-proliferative agents such as juglone to suppress the effects of nocodazole-induced mitotic arrest can be detected in a cytoblot. (a) Cartoon depiction of the ability of juglone to inhibit the effects of the cis/trans peptidyl-prolyl isomerase Pin1. As the activity of Pin1 is required for proper mitotic progression in yeast and as inhibition of Pin1 activity results in an interphase arrest, juglone treatment should prevent the entry of cells into mitosis. (b) A549 human lung carcinoma cells were seeded at a density of 4000 cells in 40 μL of DMEM+ in each well of a white, opaque 384-well plate and incubated overnight at 37° C. with 5% CO₂. Cells were either untreated or pretreated with juglone at the indicated concentrations for 8 hour. Subsequently, nocodazole at a final concentration of 250 nM was added to all wells in a final volume of 50 μL and the cells incubated for a further 12 hours. A phosphonucleolin cytoblot was performed. Equivalent concentrations of methanol had no effect on phosphonucleolin levels (data not shown). A 4 mm scale bar is shown.

FIG. 11. Cytoblots can be used to screen for small molecule suppressors of anti-proliferative agents using the presence of phosphonucleolin, and for small molecules that induce exit from mitosis using the absence of phosphonucleolin. (a) Cartoon depiction of the topoisomerase II (Top2)-dependent change in chromatin conformation required for entry into mitosis and the ability of caffeine and 2-aminopurine to suppress the effects of the Top2 inhibitor ICRF-193. (b) The ability of caffeine and 2-aminopurine to suppress the DNA damage-independent, topoisomerase inhibitor-induced G₂-checkpoint arrest can be detected in a cytoblot. A549 human lung carcinoma cells were seeded at a density of 4000 cells in 40 μL of DMEM+ in each well of an opaque 384-well plate and incubated for 24 hours at 37° C. with 5% CO₂. Cells were then either left untreated (NT) or treated with 250 nM nocodazole (ncdz), 20 μM roscovitine, 1 mM 2-aminopurine, or 2 mM caffeine, and simultaneously treated with either DMEM+ (NT), 250 nM nocodazole (ncdz), 14 μM ICRF-193, or both 250 nM nocodazole and 14 μM ICRF-193 (ICRF-193+ncdz) in a final volume of 50 μL. Cells were then incubated for 18 hours at 37° C. with 5% CO₂ and a phosphonucleolin cytoblot was performed. (c) HeLaS3 cells were seeded at a density of 4000 cells in 40 μL of DMEM+ in each well of an opaque 384-well plate and incubated for 24 hours at 37° C. with 5% CO₂. Cells were either left untreated or treated with 554 nM nocodazole for 14 hours to arrest cells in mitosis. Roscovitine was then added to the final concentrations indicated in a final volume of 50 μL and cells were incubated for 4 hours at 37° C. with 5% CO₂. Finally, BrdU was added to a final concentration of 10 μM to those wells that were assayed for BrdU incorporation, and the cells incubated for an additional 6 hours at 37° C. with 5% CO₂. BrdU and phosphonucleolin cytoblots were performed. (d) Nocodazole prevents the incorporation of BrdU and induces the accumulation of phosphonucleolin and roscovitine suppresses only the accumulation of phosphonucleolin. HeLa cells were seeded at a density of 4000 cells per well in 40 μL of DMEM+ in each well of an opaque 384 well plate and incubated overnight at 37° C. with 5% CO₂. Cells were then treated with either an equivalent amount of DMSO or nocodazole to a final concentration of 415 nM and the cells incubated for an additional 14 hours at 37° C. with 5% CO₂. Roscovitine was then added to the final concentrations indicated and final volume of 50 μL and cells incubated for 4 hours 37° C. with 5% CO₂. Finally, BrdU was added to a final concentration of 10 μM to those wells the be assayed for BrdU incorporation and the cells incubated for an additional 6 hours at 37° C. with 5% CO₂. BrdU and TG-3 cytoblots were performed (see Protocols). Wells are shown magnified 4×. (e) Caffeine and 2-aminopurine suppress a DNA damage-independent topoisomerase inhibitor-induced G2-checkpoint arrest. A549 human lung carcinoma cells were seeded at a density of 4000 cells in 40 uL of DMEM+ in each well of an opaque 384 well plate and incubated for 24 hours at 37° C. with 5% CO₂. Cells were then treated with additional DMEM+, nocodazole (250 nM), okadaic acid 100 (100 μM), caffeine (2 mM). 2-aminopurine (1 mM), roscovitine (20 uM), trapoxin (100 nM), ICRF-193 (4 μg/mL), campothecin (1 μg/mL), Hoescht 33258 (0.1 μg/mL), SB 203580 (20 μM) or juglone (667 μM) at the indicated final concentration and simultaneously treated with either DMEM+ alone, nocodazole (250 nM), ICRF-193 (4 μg/mL), or nocodazole (250 nM) and ICRF-193 (4 μg/mL) in a final volume of 50 μL. Cells were then incubated for 18 hours at 37° C. with 5% CO₂ and a TG-3 cytoblot performed (see Protocol).

FIG. 12. Screening for small molecules that affect the mammalian cell division cycle. a) Schematic of cell cycle events involved in mitotic chromosome segregation, b) summary of screening steps, c) division of small molecules into three groups based on their effects on the stability of purified microtubules.

FIG. 13. Cartoon depiction of functional fingerprinting of a test compound with 24 different antibodies. A cytoblot with 24 different antibodies is used to profile the activities of 14 different known bioactive agents (e.g. TGF-β, trapoxin, rapamycin, hydroxyurea, nocodazole etc.) in a 384 well plate. In addition, a no treatment row and a test compound row are included in the experiment. In this hypothetical experiment, we see directly the effect of the test compound on each of the 24 cellular components detected by the antibodies. For example, the cellular components detected by antibodies A, B and S are detected in untreated cells but not in cells treated with the test compound. Conversely, the cellular components detected by antibodies D, F and X are detected in cells treated with the test compound but not untreated cells. In addition to revealing this information directly, this cytoblot also allows a comparison with the known bioactive agents. Note that compound 1 and the test compound have the same profile with regard to these 24 antibodies. Thus, it is likely that the test compound and compound 1 have a similar mechanism of action. The ability to functionally categorize the test compound in this way is directly related to the number of antibodies available in the cytoblot format. However, since each antibody can divide bioactive agents into two classes (those that cause a signal with the antibody and those that do not), N antibodies can divide bioactive agents into 2^{N} classes. Thus, using just 24 antibodies, bioactive agents can be divided into more than 16 million functional categories, indicating that even a small number of antibodies is capable of providing a useful functional fingerprint of biologically active molecules.

FIG. 14 shows a schematic representation of the TGFβ signal transduction pathway.

FIG. 15 depicts various factors that participate in the TGFβ signaling pathway.

FIG. 16 presents the structure of certain preferred chemical compounds according to the present invention.

FIG. 17 presents the structure of other preferred chemical compounds according to the present invention.

FIG. 18 presents structures of four particular compounds that mimic TGFβ activity according to the present invention.

FIG. 19 graphs the dose-response of transcriptional activation of compounds 1a and 2 in the presence (*) or absence (•) of 400 pM TGFβ1. 20,000 6F mink lung cells ( ) were seeded in 384 well plates, allowed to attach for 16 hours in 10% mink medium, and were treated with the indicated concentrations of 1a or 2.

FIG. 20 depicts the reporter gene specificity of 1a and 2.

FIG. 21 shows inhibition of BrdU incorporation in mink lung epithelial cells for 1a, 2, and Cu(II).

FIG. 22 shows the effect of metal ions on activity of 1a and 2.

FIG. 23 shows activation of a TGFβ-responsive reporter gene by copper. Left panel compares activation in the presence of 64 μM 2 (•) with that in the presence of CuCl₂ (*); right panel compares activation in the presence of 10 μM ZnCl₂ (•), 100 μM Zn Cl₂ (▪), and CuCl₂ (*).

FIG. 24. Small molecules that directly affect the stability of microtubules within cells. (a) Compounds that destabilize microtubules, (b) compounds that stabilize microtubules.

FIG. 25. Additional compounds related to structure 2 of group I that also destabilize microtubules in cells.

FIG. 26. Monastrol reversibly inhibits recombinant Eg5 driven microtubule gliding in vitro. Conventional kinesin motility was not inhibited in the same assay. A. Model for spindle bipolarity. Plus-end directed motors, such as Eg5 are thought to be involved in the separation of the centrosomes and the establishment of a symmetric spindle axis. B. Inhibition of the kinesin Eg5 results in monastrol spindles. Microtubules, green; chromosomes, blue. Eg5 is depicted as homotetramer (9). C. Monastrol inhibition is reversible in vitro. After imaging Eg5 driven microtubule gliding in the presence of monastrol (200 μM), the same assay chamber was depleted of compound and the microtubule gliding recorded (Washout). At 200 μM, DHP2, a related dihydropyrimidine, does not significantly inhibit Eg5 driven microtubule gliding. D. Chemical structures of monastrol and DHP2. E. Monastrol inhibits the Eg5 driven microtubule gliding with an IC₅₀ of 14 μM. F. 200 μM monastrol does not inhibit the microtubule gliding driven by conventional kinesin.

FIG. 27 depicts particularly preferred compounds A, B, C, D and E, for use in the present invention.

### DEFINITIONS

Associated with—In certain embodiments of the present invention, a detectable entity is “associated with” a ligand. Any association that is sufficiently stable that the presence or level of the detectable entity becomes correlated with the presence or level of the ligand binding partner (i.e., with the detection target) is sufficient for the purposes of the present invention. Preferably, the association is noncovalent. However, covalent association of the detectable entity with a ligand may also be used in accordance with the present invention. More preferably, the association is electrostatic. However, additional noncovalent associations, such as hydrophobic interaction, ionic interaction, hydrogen bonding, van der Waals interaction, magnetic interaction, and combinations thereof, are also acceptable.

Biological component—Certain embodiments of the present invention involve detecting the presence or amount of a “biological component” in a reacting solution. Preferably, the biological component is detected inside a cell. A biological component may be any detectable compound or portion of a compound that (i) is found in a cell; (ii) participates in one or more biological reactions; and/or (iii) is produced by one or more biological reactions. For example, a biological component may be a protein, nucleic acid, lipid, a carbohydrate, or a complex of two or more thereof. Alternatively or additionally, the biological component may be an atom (such as a phosphate that is added to a protein as a result of a biological reaction), a moiety (such as a carbohydrate group), a metal, a salt, or even a three-dimensional structure (e.g., a conformational epitope recognized by an antibody).

Biological reaction—A “biological reaction”, as that term is used herein, means a reaction that occurs in nature, preferably one that occurs inside a living cell. For the purposes of the present invention, the biological reaction may be reproduced in a reaction vessel in a context different from that in which the reaction occurs in nature. For example, a reaction that occurs inside of a cell in nature may be reproduced in the absence of cells (e.g., in a cell extract) in the inventive system. However, it is generally preferred that the biological reactions employed in the practice of the present invention occur inside cells.

Detection target—The “detection target” is the compound or entity whose detection reveals the effect(s) of the test compound(s) on the reaction(s) of interest. Typically, but not necessarily the detection target will be a product of or participant in the reaction being studied. Any compound or entity whose presence or level can be correlated with an event of interest may be selected as a detection target.

Library—In general, a “library” of chemical compounds is any collection of compounds. However, the term “library” is also used in a more specific context to mean the collection of compounds that is produced in a particular combinatorial synthesis. Which meaning of the term applies in any particular case will be readily apparent from context.

Reacting solution—A “reacting solution” is any solution undergoing one or more chemical or biological reactions. The solution may be aqueous or organic but for the purposes of the present invention is preferably aqueous. It is particularly preferred that the solution contain one or more cells and that the reaction of interest be taking place within the cell(s).

Reaction—It will be understood that the term “reaction”, as used herein, includes but is not limited to processes through which a substrate is chemically modified to produce a product. Any biological or chemical process or event may be considered to be a reaction in accordance with the present invention. To give but a few illustrative examples, DNA replication, protein phosphorylation, cell division, signal transduction, gene expression, etc. may all be considered reactions as that term is used herein.

Reaction vessel—A “reaction vessel”, as that term is used herein, is any container that can containing a reacting solution. For example, test tubes, petri dishes, and wells can all constitute reaction vessels. Preferably, a reaction vessel is a well in a multiwell plate or other multivessel format.

Specific binding—According to the present invention, a ligand “binds specifically” to a detection target if it discriminates between that detection target and other components present during the period of contact between the ligand and the detection target. Typically, the ligand will need to be able to discriminate between the detection target and other components of the reacting solution. In preferred embodiments, the ligand has a strong affinity for the detection target, reflected in a Kd less than or equal to approximately 10⁻⁶, preferably less than or equal to approximately 10⁻⁹. The affinity of the ligand for any other component of the solution in which the ligand contacts the detection target should not be greater than the affinity of the ligand for the detection target. Preferably the affinity of the ligand for any other component does not have a dissociation constant smaller than a Kd of approximately 10⁻³. More preferably, the affinity of the ligand for any other component does not have a dissociation constant smaller than a Kd of approximately 10⁻⁴. Most preferably, the affinity of the ligand for any other component does not have a dissociation constant smaller than a Kd of approximately 10⁻⁵. In some cases, the ligand comprises two or more molecules that are or become non-covalently associated with one another (e.g., a primary and secondary antibody). Under such circumstances, the interaction of the two or more molecules should be sufficiently specific and/or stable that the combination meets the requirements of a ligand, as defined herein, under the conditions in which the two or more molecules are contacted with its binding partner. For example, where the ligand comprises primary and secondary antibodies, it is possible that the secondary antibody will be contacted with the first antibody under very different conditions that those under which the primary antibody was contacted with the detection target (e.g., after several washes). Thus, the secondary antibody need only be able to bind specifically to the first antibody under the conditions in which it is contacted with that antibody; it is immaterial whether the secondary antibody could have reacted specifically with the first antibody under the conditions of contact between the first antibody and the detection target.

Test compound/test chemical—The terms “test compound” and “test chemical” are used herein to refer to chemical compounds whose function(s) is are/to be assayed through the practice of the present invention.

### DESCRIPTION OF CERTAIN PREFERRED EMBODIMENTS

The present invention provides a system for high-throughput analysis of chemical compounds. Assays are performed in a high density platform, and compounds having pre-determined desirable effects are identified. Preferably, the compounds have biological effects, more preferably, the assays and detection are performed on whole cells. Various elements of the inventive system are discussed more fully below.

### Platform

Assays may be performed in any of a variety of high-throughput platforms according to the present invention. It is generally desirable that many assays be performed simultaneously, preferably in small volumes. Given that standardized instrumentation is available for performing manipulations of multi-well trays of particular dimensions, such trays are preferred for use in practicing the inventive methods.

Miniaturization of reaction vessels saves reagent costs and also increases the number of reaction vessels that can be incorporated into a particular platform. Preferably, reaction vessels hold about 200 microliters, more preferably reaction vessels hold approximately 50 microliters, still more preferably reaction vessels hold approximately 2 microliters, and most preferably about 250 nanoliters. For biological assays of whole cells, of course, it is necessary that the reaction vessels be sized to accommodate at least one cell, preferably at most 8000 cells, more preferably at most 2000 cells, even more preferably at most 500 cells, and most preferably at most 100 cells.

Preferably, the cells used reaction vessels described in the preceding paragraph are mammalian cells. However, any biological or chemical system may be utilized in the reaction vessels in accordance with the present invention. For a non-limiting example of another biological system, other cells such as bacteria, yeast, plant and insect cells may be used. The number of cells for these example that are used in miniaturized reaction vessels will differ from mammalian cells depending on the size of the cells.

According to the present invention, assays are preferably performed in dense arrays of reaction vessels. Preferably, the center-to-center distance between reaction vessels is less than about 8.5 mM. More preferably, the distance is less than 4.5 mM. Even more preferably the distance is less than approximately 2.25 mM. Most preferably, the distance is less than approximately 1 mM.

Of course, conventional high throughput screens are often performed in commercially available 96-well plates (see, for example, Rice et al. Anal. Biochem. 241:254-259. 1996). Such plates may be utilized according to the present invention. However, denser arrays are generally preferred, though it is appreciated that such arrays may desirably have the same external dimensions of a standard 96 well plate in order to facilitate automation using available equipment. Plates containing 384 (Nalge Nunc International, Naperville, Ill.; Greiner America, Lake Mary, Fla.; Corning Costar, Corning, N.Y.) or 1536 (Greiner America, Lake Mary, Fla.) wells have recently become commercially available and may be used in the practice of the present invention. Still denser plates, such as the 6144 well plates described by You et al. (Chem. Biol. 4:969-975. 1997; U.S. Ser. No. 09/184,449 entitled “Casting of Nanowell Plates” each of which is incorporated herein by reference) are particularly preferred. An ideal assay for high throughput screening would be compatible with any or all of these array formats.

### Assay

Any assay of interest may be performed with the inventive system. Preferably, the assays provide information on biological activities of the compounds under analysis. More preferably, the assays utilize whole cells. Any cells can be used including, for example, bacterial cells, yeast cells, plant cells, insect cells, or animal cells. Preferred are mammalian cells, more preferred are human cells. Also, in certain preferred embodiments, the cells are part of an intact tissue or organism.

Particularly preferred intact organisms that can be assayed in accordance with the present invention include, for example, the nematode Caenorhabiditis elegans, the fruit-fly Drosophila melanogaster and embryos of the frog Xenopus laevis and the zebrafish Danio rerio. One advantage of using a whole organism is the ability to assay for phenotypes that are specific to tissues or developmental processes and behavior. For example, one could screen for small molecules that induce tissue differentiation or organ formation (with a specific biochemical marker of the differentiated cell type) including but not limited to appendages, eyes, bone, liver, pancreas, heart, lung, brain, intestine, pharyngeal muscle. One could also screen for small molecules that affect feeding behavior, fat cell accumulation, mating, longevity, or motility.

In particularly preferred embodiments of the invention, the assays employed detect an event that occurs inside cell or organisms. For example, preferred embodiments of the invention involve detection of the presence or amount of an intracellular biological component. Often, detection of the presence or amount of such a biological component will reveal a perturbation in an underlying biological process.

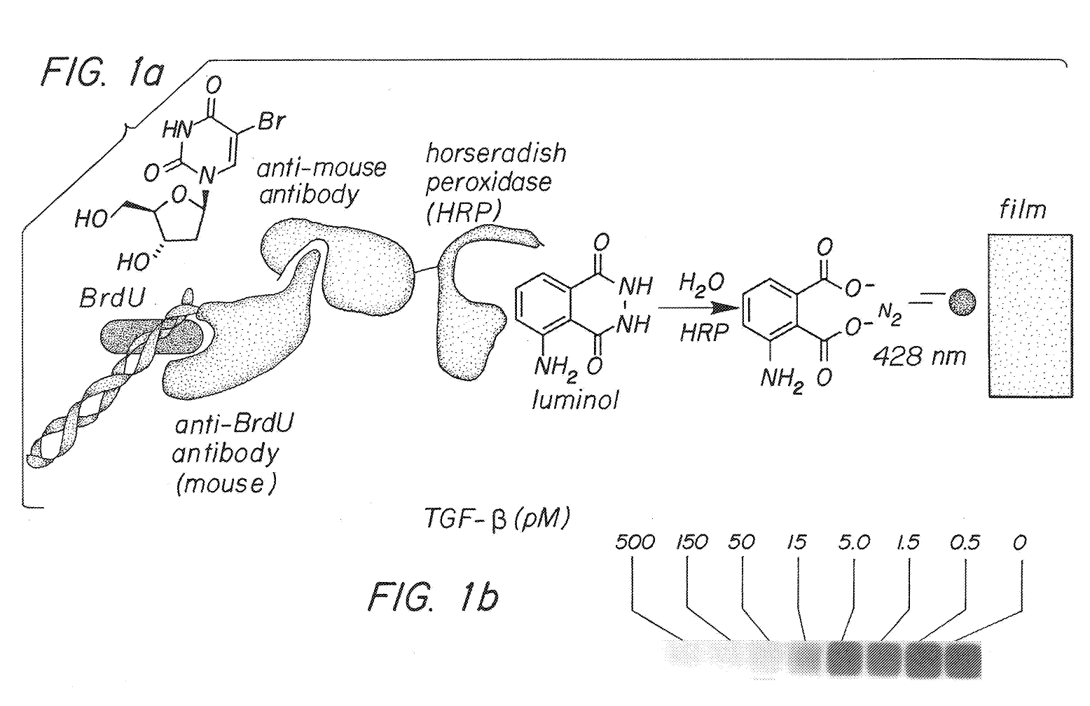

To give but a few non-limiting examples, the biological component may be an indicator of cell growth and viability, so that test compounds may be screened for their ability to disrupt one or more processes involved in maintaining cell viability. Preferred biological components for such assays include compounds, such as a natural or non-natural nucleotide, that is incorporated into the DNA of replicating cells. Detection of an increase or decrease in the amount of such a compound that is incorporated into cells in the presence of a test compound as compared with cells not exposed to the compound allows the identification of compounds that perturb cell proliferation processes, including DNA replication. 5-bromodeoxyuridine (BrdU) is an analog of thymidine in which the methyl group at the 5-position is replaced with a bromine (see FIG. 1a). When provided to replicating cells, this analog is efficiently incorporated into their DNA. As described in Examples 1-3, we have demonstrated that the inventive system may be employed to detect BrdU inside living cells, and therefore to screen test chemicals for their effects on cell replication.

Alternatively or additionally, the biological component may be a component or product of a cell signaling pathway, so that detection of the component allows the identification of test compounds that perturb the pathway. For example, the inventive system may be employed to identify compounds that perturb mitogen signaling pathways. Many mitogens (e.g., insulin, platelet-derived growth factor, interleukin-2, etc.) induce phosphatidylinosital 3-kinase (PI3K) activity within the cells that they stimulate. Action of PI3K produces the second messenger phosphtidylinositol 3,4,5-triphosphate (PIP3), which could be a detection target of an inventive assay. Other second messengers could similarly be detected to allow the identification of compounds that perturb other signaling pathways.

In certain preferred embodiments, the biological component used as a marker for a cell signaling pathway is a moiety that is covalently attached to a protein or other molecule during operation of the signaling pathway. Many therapeutically important signaling pathways including, for example, cell cycle progression, gene expression, and determination of cell fate, involve covalent modification of cellular proteins, so this approach can be applied to any of a variety of specific biological processes. In particular, we have utilized the inventive system to detect acetylation of histone H4 inside living cells. Histone acetylation and deacetylation is a mechanism by which cells modulate transcription (Example 4). We have also used the inventive system to detect phosphorylation of histone H3 and nucleolin (see Examples 4 and 5). Both of these proteins are phosphorylated during mitosis, so that detection of their phosphorylated forms allows the identification of test compounds, for example, that arrest cells in mitosis or that inhibit DNA da mage-induced G₂ checkpoint. Of course, detection of these phosphorylated proteins, or histone acetylation, may also be used as general markers of cellular state, allowing the identification of test compounds that indirectly induce these molecular changes.

As but another example, the biological component may be a marker for cell differentiation. For example, insulin is a marker for β-islet cells of the pancreas (Ally et al., J. Immunol. 155:5404-5408, 1995) and intestinal fatty acid binding protein is a marker for the jejunum (Playford et al., Proc. Natl. Acad. Sci. USA 93:2137-2142, 1996). Also, histone H4 acetylation, discussed above as a marker of gene expression, is also associated with cell differentiation and could alternatively or additionally be used to monitor differentiation processes. For assays of cell differentiation, it is generally desirable to employ cells that undergo differentiation. Embryonic stem (ES) cells are particularly preferred in this regard.

In yet another non-limiting example, the biological component may be a marker for gene expression, including for example, a product of such expression. For example as described in Example 14, we have used the inventive system to identify compounds that mimic TGF-β in that they stimulate expression of TGF-β-responsive genes. The biological component that we detected was the product of a luciferase reporter gene.

In yet another non-limiting example of a chemical system, compounds may be screened in accordance with the present invention to identify catalysts of chemical reactions. The concentration of reactants and/or products of a chemical reaction may be directly or indirectly detected using a ligand that binds to a reactant and/or a products of the chemical reaction to detect reactants and/or products.

It will be appreciated that more than one assay can be performed together, so that complex information about reaction behavior is obtained. To give but one non-limiting example, simultaneous analysis of H4 acetylation as a marker for differentiation and BrdU incorporation as an indicator of cell replication, can be used to identify chemical compounds that perturb the proliferation of differentiated cells, including compounds that induce such proliferation.

In general, the inventive assays involve contacting a reacting solution (i.e., a solution in which one or more reactions is/are occurring) with one or more test compounds, and detecting an effect (or lack thereof) of the test compound(s) on the reaction of interest. Preferably, a plurality of reacting solutions is arrayed in a high-throughput format containing multiple reaction vessels as described above, and different compounds are introduced into each vessel. The different effects of all of the different compounds on the reaction may then be simultaneously determined. Also, individual test compounds may be studied in a variety of different assays, so that a functional “fingerprint” of their activities is obtained.

In general, test compounds may be delivered to the reaction vessels by any mechanism that achieves their deposition in the vessels. For example, test compounds may be individually aliquoted into the vessels. Alternatively, a collection of compounds may simultaneously be delivered to a plurality of vessels, for example using a pin array, or a multi-point syringe or pipette. In particularly preferred embodiments, delivery of compound is automated.

Compounds may be delivered prior to, during, or after introduction of the reacting solution to the vessel. The amount of time that compounds and reacting solution are maintained in contact with one another may depend on the particular assay being performed. For example, where the assay is one that analyses DNA synthesis in living cells (e.g., via detecting incorporated BrdU), it is generally desirable to maintain the compounds in contact with the cells while the cells undergo at least one round of replication (e.g., by maintaining the compounds in contact with the cells for a period of time long enough to include at least one division cycle for cells that are not in contact with a test compound). Alternatively or additionally, where a reference compound is known and efforts are being made to identify a test compound with similar activity to the reference compound, the test compound is usually contacted with the reacting solution under conditions in which the reference compound is known to display its activity.

### Detection

Any available detection system may be used to assess a test compound's effect on the reaction(s) of interest. Preferably, the presence of a reaction product or participant is detected through the use of a ligand that binds specifically to the product or participant and is associated with a detectable entity. Any molecular compound that can bind specifically under the detection conditions may be employed in the present invention. Non-limiting examples of such molecules include proteins, peptides, amino acids, nucleic acids, lipids, porphryins, synthetic compounds such as Hoescht 33258, DNA-binding ruthenium complexes (Murphy and Barton. Methods Enzymol 1993. 226:576-94) and methidium propyl EDTA, synthetic peptides (Wade et al. Biochemistry. 1993 26; 32(42):11385-9), peptide nucleic acids (Nielsen. Curr Opin Struct Biol 1999 June; 9(3):353-7), antibodies, polyclonal antibodies, monoclonal antibodies, non-natural amino acid derivatives (Thorson et al. Methods Mol. Biol. 1998; 77:43-73), non-natural nucleic acid derivatives, molecules involved in signal transduction, biotin, avidin, streptavidin, magnetic compounds, molecules that bind to carbohydrates, molecules that interact and bind with lipids, and precursors of these examples.

The ligand may comprise a single molecule or compound, or may comprise multiple molecules or compounds, at least one of which can bind specifically to the selected detection target. For example, the ligand may comprise a first binding partner that binds specifically with the detection target and a second binding partner that binds specifically with the first binding partner. Those of ordinary skill in the art will appreciate that a wide variety of established specific associations are known in the art that could be employed in a first binding partner/second binding partner ligand. To give but a few non-limiting examples, the first binding partner/second binding partner interaction may involve primary and secondary antibodies, biotin/avidin, nucleic acid/nucleic acid, nucleic acid/intercalation compound (e.g., DAPI, methidium propyl EDTA (MPE), ruthenium complexes), protein/nucleic acid complexes, protein-protein complexes, protein/small molecule interactions (i.e. inhibitors of enzymes such as reverse transcriptase, DNA polymerase, RNA polymerase), protein/carbohydrate interactions, protein/lipid interactions, carbohydrate/carbohydrate interactions, molecules that bind to glycoproteins, and FK506/rapamycin,

Alternatively or additionally, the ligand may comprise a first binding partner that is also a modifying agent, so that the detection target becomes chemically altered as a result of its interaction with the first binding partner. The ligand may then comprise a second binding partner that detects the modification. Other variations on ligand composition will be apparent to those of ordinary skill in the art.

In preferred embodiments of the invention, the ligand comprises an antibody to the detection target. Often, the ligand will comprise a primary antibody to the detection target and a secondary antibody to the primary antibody. In general, ligand antibodies may be monoclonal or polyclonal, but monoclonal are generally preferred, particularly for antibodies to reaction products or participants.

In other preferred embodiments of the invention, the ligand comprises a polynucleotide, comprised of natural nucleotides (A, T, G, C, and U), and/or nucleotide analogs or derivatives, that hybridizes specifically with a target sequence in the reacting solution.

The detectable entity may comprise any compound, complex, or process, that can be detected under the conditions of the inventive assay. For example, the detectable entity may be or may produce a compound that is radioactive, fluorescent, phosphorescent, chemiluminescent or absorbs and/or emits radiation in the UV-IR spectrum. Use of radioactivity offers a high degree of sensitivity but creates complicated issues associated with handling and disposal of materials. Chemiluminescence is particularly preferred for use in the practice of the present invention. The advantages of using chemiluminescent detection include sensitivity, safety (since no radioactivity is used), accuracy, speed (since detection of luminescence can be performed in seconds to minutes) and convenience.

Fluorescent compounds that may be detected according to the present invention include green fluorescent protein, and a variety of commercially available fluorescent dyes (see for example, “Handbook of Fluorescent Probes and Research Chemicals.” Haugland. Molecular Probes. Eugene Oreg. Incorporated herein by reference.) In addition, applications utilizing fluorescent quantum dots may be included in the present invention. (Bruchez et al. Science 281:2013-2016; Chan and Nie. Science 281:2016).

In one particular preferred embodiment of the present invention, the detectable entity comprises a peroxidase that catalyzes a chemiluminescent reaction. For example, a variety of chemiluminescent substrates are available for horse radish peroxidase (HRP) Preferred for use in the practice of the present invention are diacylhydrazides, such as luminol. Diacylhydrazides are oxidized in the presence of hydrogen peroxide, and luminesce to emit photons. The luminescence resulting from the oxidation of luminol can be enhanced using a phenol derivative, preferably 4-iodophenol (ECL™; Nycomed Amersham Corporation, Buckinghamshire, England). The luminescence can then be detected by film, detected using photomultiplier technology or detected by a charge-coupled device attached to a camera and/or a computer. The use of luminol as an HRP substrate greatly enhances the sensitivity of detecting HRP relative to other substrates such as color dyes (e.g. o-phenylenediamine; OPD). This increased sensitivity of detection allows for small sample sizes. FIG. 1 presents a schematic representation of but one particular preferred embodiment of the present invention, in which HRP is coupled to a secondary antibody, used to detect a primary antibody that interacts with a detection target.

The detection systems and formats described herein are sufficiently sensitive that detection of the detection targets is approximately 10 fold more sensitive with HRP and chemiluminescence as compared to colorimetric methods of detecting HRP (ECL™; Nycomed Amersham Corporation, Buckinghamshire, England). In addition, according to the manufacturer, an ECL Plus™ system utilizing an acridan-based substrate that releases a high level, sustained output of light can give a 4 to 20-fold increase in sensitivity as compared with ECL detection. The ECL Plus™ system may also be used in accordance with the present invention. Furthermore, chemiluminescent systems that use HRP and luminol with an enhancer other than 4-iodophenol may be used in the present invention (e.g. Pierce Chemicals)

It will be appreciated that two or more different detection targets may be assayed simultaneously in accordance with the present invention, for example through the use of two or more different ligands associated with detectable entities.

### Test Compounds

Any collection of chemical compounds may be assayed in the inventive system. Compounds may be obtained from natural or synthetic sources. To date, few synthetic chemical compounds have been identified that bind to biological targets and exert effects on biological processes; generally, only compounds isolated from natural sources (see, for example, Hung et al., Chem. Biol. 3:623-640, 1996) have been shown to have such effects. One advantage of the present invention is that it provides a sensitive, high-throughput system that allows the identification of synthetic chemical compounds that perturb biological processes. With the advent of combinatorial synthetic chemistry techniques (see, for example, Borman, Chem. Eng. News Feb. 24, 1997. pp. 43-62; Thompson et al., Chem. Rev. 96:555-600, 1996), a large number of synthesized “libraries” of chemical compounds have become available (see, for example, U.S. Ser. No. 09/121,922; U.S. Ser. No. 60/114,909; US National application filed Jun. 10, 1999 entitled “Biomimetic Combinatorial Synthesis” claiming priority to U.S. Ser. No. 60/089,124 filed on Jul. 11, 1998; all of which are incorporated herein in their entirety by reference.) Some of these are “natural-product-like” in that they contain compounds with complex structures similar to those found in natural products (see, for example, US National application filed Jun. 10, 1999 entitled “Biomimetic Combinatorial Synthesis” claiming priority to U.S. Ser. No. 60/089,124 filed on Jul. 11, 1998). Any or all such libraries can be screened in accordance with the present invention.

Test compounds may be attached to a solid support or may be free in solution. Of course, where it is desired that a test compound enter a cell, it is generally preferred that the compound not be attached to a support. However, the compound may be delivered to the reaction vessel in association with a support, and be released from the support inside the vessel. As is well known, combinatorial libraries are often synthesized on solid supports, which typically contain encoding information enabling the rapid identification of the particular synthesized compound that is attached to the bead (see, for example, Czamik, Curr. Opin. Chem. Biol. 1:60-66, 1997).

Often, it will be desirable to screen a large library, for example under moderate stringency, to identify molecules within the library that are likely candidates of interest, and subsequently to prepare sub-libraries, or related libraries (e.g., by combining different compounds or performing new syntheses), that can be screened at higher stringency. This approach may be iterated as often as desired.

### Characterization of Identified Compounds

### Target Identification

Once a new chemical compound with biological activity of interest is discovered, whether by screening natural products or combinatorial libraries, it may be desirable to elucidate its mechanism of action. For example, radiolabelled versions of the compound may be prepared, and the molecular targets of the compound can then be identified because they become associated with radioactivity by virtue of their interaction of with the compound. In some cases, cross-linking or other studies may be performed to attach the radioactivity to the target covalently.

Alternatively or additionally, interacting targets may be identified biochemically, for example by fractionating cellular extracts with an affinity matrix containing a derivative of the biologically active agent. These methods have worked well to identify the molecular targets of biologically active natural products, but are time-consuming.

An alternative approach for identifying the interaction target of a test compound utilizes a “three-hybrid” transcriptional activation system, in which an anchored derivative of a chemical compound is displayed against a library of cDNAs fused to a transcriptional activation domain (Borchardt et al. Chem. Biol. 4:961-968. 1997; Licitra & Liu. Proc Natl Acad Sci USA 93:12817-12821, 1996). Another method involves the use of small-pool expression cloning (King et al. Science 277:973-974, 1997). A third approach to determining the mechanism of action of identified chemical compounds of interest involves the use of oligonucleotide or cDNA microarrays. In this method, the concentration of numerous cellular mRNAs is detected in parallel by hybridization to a microarray of cDNAs or oligonucleotides (Schena et al. Science 270:467-470. 1995).

### Functional Fingerprinting

In one particular embodiment of the present invention, test compounds are characterized by their multiple effects on a cell. For example, it is already known that small molecules can be “fingerprinted” by the pattern of changes that they induce in the transcriptional profile of a cell (Myers et al. Electrophoresis 18:647-653, 1997). Such transcriptional profiling may be performed for test compounds identified according to the present invention. However, while mRNA fingerprinting in this manner is a powerful tool, many cellular events, including all post-transcriptional events, cannot be detected with this method.

The present invention provides a system whereby chemical compounds can be fingerprinted based on the changes that they induce in a variety of different cellular processes, including, for example, protein concentration, phosphorylation, methylation, acetylation, lipidation, isoprenylation, ubiquitination; second messenger concentration; and the rate or extent of DNA synthesis. The total pattern of these alterations constitutes an effective “fingerprint” (i.e. biological profile) of each bioactive agent. Example 12 describes one embodiment of functional fingerprinting according to the present invention.

### Formulations and Uses of Identified Compounds

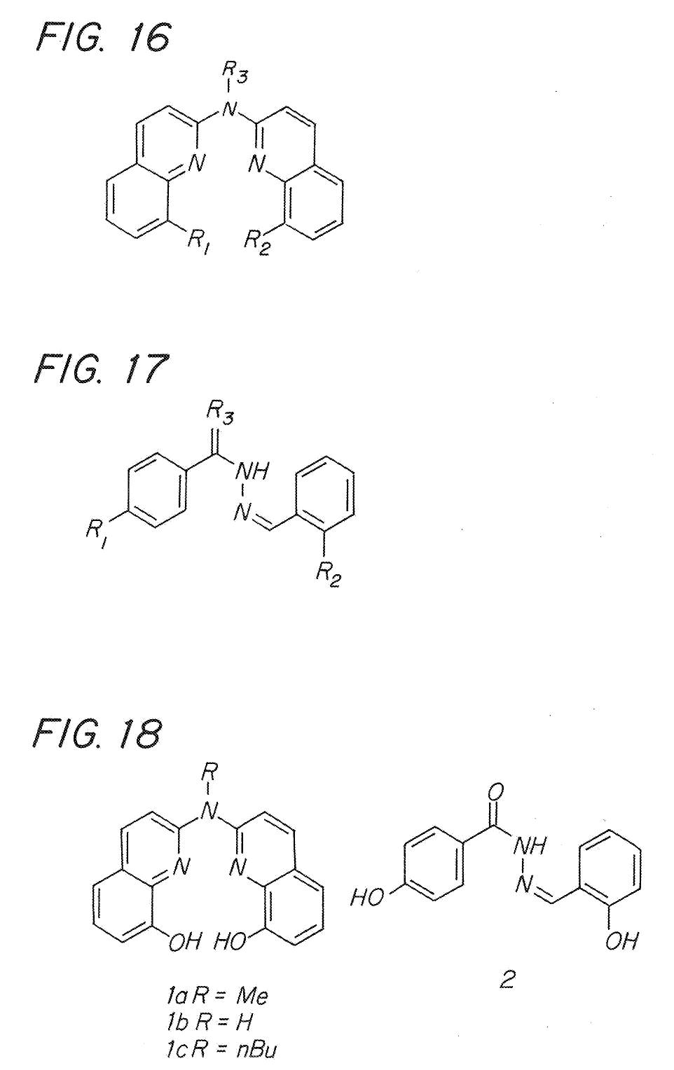

The present invention also provides biologically or chemically active compounds identified through use of the inventive system. To give but one example, as described in Example 14, we have used the inventive system to identify compounds that mimic TGF-β in that they stimulate expression of TGFβ-responsive genes. In particular, the compounds have the structure depicted in FIG. 16, where each of R1 and R2 is selected from the group consisting of hydroxy, methoxy, alkoxy, amino, and thiol groups and R3 is selected from the group consisting of linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and any derivative incorporating phosphorous. R1 and R2 may be the same or different. Preferably, R1 and/or R2 are hydroxyl groups (—OH). R3 is preferably an alkyl group, more preferably a short (≦about 5 carbon, preferably ≦about 3 carbon)-chain alkyl group or H. In particularly preferred embodiments, R3 is selected from the group consisting of nBu, Me, and H.

These compounds are characterized by an ability to stimulate expression of genes under the control of TGFβ-responsive elements in a dose-dependent manner, and also by an ability to inhibit BrdU incorporation into DNA. Preferably, the compounds increase gene expression at least approximately 2-fold, more preferably at least approximately 4-fold, 5-fold, 10-fold, 50-fold, or 100-fold, as compared with the level of expression observed in the absence of the compounds. The compounds preferably bind to one or more transition metals, preferably including zinc. Certain preferred compounds activate transcription in yeast of one or, ore genes encoding a metal binding or metal transporting protein.

The present invention also provides compounds having the structure depicted in FIG. 17, where each of R1 and R2 is selected from the group consisting of linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and any derivative incorporating phosphorous, and R3 is selected from the group consisting of carbonyl, sulfonyl, and hydroxyl groups. R1 and R2 may be the same or different. Preferably, R1 and/or R2 are hydroxyl groups (—OH). R3 is preferably a carbonyl group (═O).

Any formulation of the inventive compounds is a “composition” according to the present invention. Any inventive composition that is formulated for delivery to a living organism is considered a “pharmaceutical composition” according to the present invention. Inventive compositions include compositions formulated with one or more pharmaceutically acceptable carriers as is known in the art, and/or with other binders, solvents, surfactants, etc. that one of ordinary skill in the art will understand would be useful to prepare a pharmaceutical composition for use in accordance with the present invention. Such pharmaceutical compositions may be formulated for any mode of delivery, including but not limited to injection, inhalation, transdermal passage, ocular, vaginal or rectal delivery, or swallowing.

Alternatively or additionally, compositions may be formulated for use as reagents in in vitro or in vivo reactions.

The inventive compounds and compositions may be employed for any of a variety of purposes in accordance with the present invention. Any application that exploits their biological or chemical function identified as described herein is considered to be within the scope of the present invention.

For example, the metal binding compounds that activate TGFβ-responsive genes may be employed for any of a variety of purposes in accordance with the present invention. Any application that exploits their ability to bind metals and/or their ability to mimic TGFβ activity is considered to be within the scope of the present invention.

To name just a few examples, they may be employed to stimulate expression of one or more TGFβ-responsive genes, and/or to alter local concentrations of one or more transition metals. To the extent that they mimic TGFβ, they may be employed as immunosuppressive agents (similar to cyclosporine, which is thought to extent its immunosuppressive effects at least in part through activation of the TGFβ pathway), or may alternatively be used as anti-inflammatory agents. Their ability to activate TGFβ-responsive genes, including those that block cell proliferation, makes them attractive anti-cancer agents. Similarly, they may be used as coagulation/wound healing agents due to their ability to activate genes such as PAI-1.

The ability of the inventive compounds to bind and/or transport metals creates additional contexts for their application independent of or in addition to their ability to activate TGFβ-responsive genes. Failure to maintain appropriate metal ion levels is associated with a range of human diseases, including neurodegeneration, metal ion overload or deficiency states, and metal ion storage diseases. Menkes and Wilson's diseases, for example, both result from defects in copper transporters. Inventive compounds that transport copper are particularly useful to treat these diseases. To the extent that inventive compounds transport iron, they may be useful in the treatment of anemia.

### Chemical Genetics

As will be appreciated by those of ordinary skill in the art, the inventive system may be employed to detect chemical compounds will any desired chemical or biological activity. In one particularly preferred embodiment, the system is employed to identify chemical reagents that perturb biological processes and therefore may be used as probes to facilitate the dissection and analysis of those processes.

Those of ordinary skill in the art will readily appreciate that genetic strategies have proven profoundly useful in the analysis of biological processes. Traditional modifier screens seek to identify gene products that, when altered or mutated, suppress or enhance a previously identified alteration or mutation in a pathway. Such screens, referred to as suppressor and enhancer screens, have proved powerful tools for the elucidation of gene function in genetically tractable model organisms such as the budding yeast Saccharomyces cerevisiae, the nematode Caenorhabiditis elegans, and the fruit-fly Drosophila melanogaster. The advantage of a suppressor/enhancer screen over simply starting with wild-type conditions and screening for additional mutants with the same phenotype is the possible identification of otherwise unrecoverable mutations or in sensitizing the pathway to further perturbation.

Unfortunately, such suppressor/enhancer screens generally cannot be performed in mammalian systems. In part this is due to the long generation time, expense and requirements for such analysis of whole organisms. More significantly, while a limited number of dominant mutations that result in a phenotypic effect when only one copy of a locus is inactivated have been identified, it has not been possible to efficiently generate homozygous mutations in autosomal recessive genes as the presence of an unmutated copy of the majority of genes precludes phenotypic detection of mutations in other alleles because of the requirement that the function of all copies of a locus be mutated in order to observe phenotypic effects. While it is possible to accomplish this end through the use of selection, homologous recombination and the mating of heterozygous animals or the use of antisense RNA-mediated inhibition, such approaches require prior knowledge of the DNA sequence of the target gene sequence and thus are not applicable to the elucidation of novel components of pathways. Finally, as many gene products are necessary for cell viability or are expressed in a restricted tissue or temporally-specific manner, the analysis of these gene products in mammalian systems requires a method of conditional alteration or inducibility of a gene product.

The present invention provides a system through which small molecules are utilized as both the potential source of the starting gene product alteration and/or the source of variation which is selected from in order to identify inhibitors/activators of known or novel components of signaling pathways. This approach has the potential to overcome many of the current limitations to genetic analysis in mammalian model systems. For example, due to their mechanism of application and action, 1) relevant small molecules can act analogous to a dominant mutation or a homozygous recessive mutation insofar as it can specifically alter or eliminate the function of gene products from all alleles of gene; and 2) the alteration in the gene product is entirely conditional and could additionally be reversible or irreversible depending on the nature of the interaction. Thus, by analogy to suppressor or enhancer screens of genetic analysis, the development of a general approach for screening small molecule libraries for suppressors and/or enhancers of a specific phenotypic effect that is due to the effect of additional alterations or mutations allows for the elucidation and characterization of compounds that perturb specific cellular processes. Moreover, identification of the relevant intracellular target of these compounds can reveal the existence of novel genes on signaling pathways of interest in mammalian cells. As this approach does not require the use of engineered cell lines per se, this means a wide range of cells including both primary and transformed cells of any tissue type or genetic background can easily be used and compared. Example 13 describes one particular embodiment of the use of the present system in a chemical genetics assay.

### EXAMPLES

The following examples illustrate certain preferred modes of making and practicing the present invention, but are not meant to limit the scope of the invention since alternative methods may be used to obtain similar results.

### Example 1

### Detecting Changes in DNA Synthesis in a Small Sample of Cells

The ability of the cytoblot assay to detect changes in DNA synthesis was tested by measuring the incorporation of 5-bromodeoxyuridine (BrdU, Sigma Corporation, St. Louis, Mo., Cat.# B9285) into DNA in the presence or absence of the anti-mitogen transforming growth factor β (TGF-β). Mink lung cells, which are responsive to TGF-β, were seeded into each well of an opaque, white 384-well plate. The cells were subsequently treated with varying concentrations of TGF-β for 16 hours and then with 10 μM BrdU for 16 hours. FIG. 1b shows that TGF-β treatment effectively prevented BrdU incorporation and that background staining in the presence of TGF-β was negligible. The following protocols provide experimental detail.

### Protocol for 384 Well BrdU Cytoblot

The reagents to be tested were prepared in 384 well plates with one or more test compound per well. If the test compounds were attached on solid support (beads), then the beads were distributed into individual wells of a clear-bottom 384 well white plate (Corning Costar, Corning, N.Y., Cat.# 3707) with a Multidrop 384 plate filler (Lab Systems, Helsinki, Finland) in acetonitrile. Compounds were then released by photolysis or chemical treatment with some or all of that acetonitrile solution transferred to a new 384 well white-bottom plate (Nalge Nunc International, Naperville, Ill., Cat.# 164610). The organic solvent was evaporated off. Alternatively, if a concentrated stock solution was available, the compound was transferred into the test plate after the cells were seeded using small pins, syringes or pipettes to deliver approximately 50-500 nL. The cells were seeded (40 μL per well, 2000 Mv1Lu (American Type Culture Collection (ATCC), Rockville, Md., Cat. # CCL64) mink lung epithelial cells) in the presence or absence of a known biological agent (e.g. 200 μM TGF-β1, Sigma Corporation, St. Louis, Mo., Cat.# T-1654).

After approximately 16 to 36 hours, 10 μL of 50 μM BrdU (Sigma Corporation, Cat.# B9285) was added to each well (with the Multidrop 384) for a final concentration of 10 μM BrdU. The cells were incubated for an additional 4 to 16 hours.

The plates were cooled on ice for 15 minutes. The plates were kept under aluminum foil, and exposure to light was minimized for all of the remaining operations. The supernatant was removed from each well with a 24 channel wand (V&P Scientific, San Diego, Calif.) attached to a vacuum source. This wand was used for aspiration throughout the protocol.

50 μL of a cold (4° C.) solution of 70% ethanol/30% phosphate buffered saline (PBS) was added to each well. The plates were incubated one hour on ice. The ethanol/PBS solution was aspirated off and 90 μL of cold (4° C.) PBS was added to each well. The PBS solution was aspirated off and 25 μL of 2 M HCl/0.5% tween 20/H₂O was added. The plates were incubated at room temperature for 20 minutes. The acid was aspirated off and 90 μL of a solution of 10% 2M NaOH/90% Hank's Balanced Salt Solution (HBSS, GibcoBRL, Gaithersburg, Md. Cat.# 24020-117) was added to each well. The base solution was aspirated off and 90 μL of HBSS was added to each well. The HBSS was aspirated off and an additional 90 μL of HBSS was added to each well. The HBSS was then aspirated off and 75 μL of PBSTB (PBS; 0.1% Tween 20 (Sigma Corporation, Cat.# P-1379), 0.5% bovine serum albumin (Sigma Corporation, Cat.# A-2153)) was added to each well.

The PBSTB was removed and 20 μL of antibody solution was added. Antibody solution contained 0.5 μg/mL mouse anti-BrdU antibody (1:1000 dilution of stock, Pharmingen, San Diego, Calif., Cat.# 33281A) and a 1:2000 dilution of anti-mouse Ig antibody conjugated to HRP (Amersham Corporation, Cat.# NA9310) in PBSTB. The plates were incubated for one hour at room temperature. The antibody solution was aspirated off and 90 μL PBS was added to each well. The PBS solution was aspirated off and another 90 μL PBS was added to each well. The PBS solution was aspirated off and 20 μL HRP substrate solution was added to each well. The HRP substrate solution was obtained by mixing equal volumes of solutions 1 and 2 from the Amersham ECL detection kit (Cat.# RPN₂₁₀₆).

The plate was allowed to incubate for five minutes at room temperature. Then the plate was placed on a flat surface in a dark room. A piece of film (X-OMAT AR, Kodak Corporation, Rochester, N.Y.) was placed on top of the plate. Exposures of one minute and 5 minutes were usually sufficient for detecting BrdU activity in mink lung cells. Longer or shorter exposures can be made. The film was developed in a Kodak M35A X-OMAT processor (Kodak Corporation, Rochester, N.Y.).

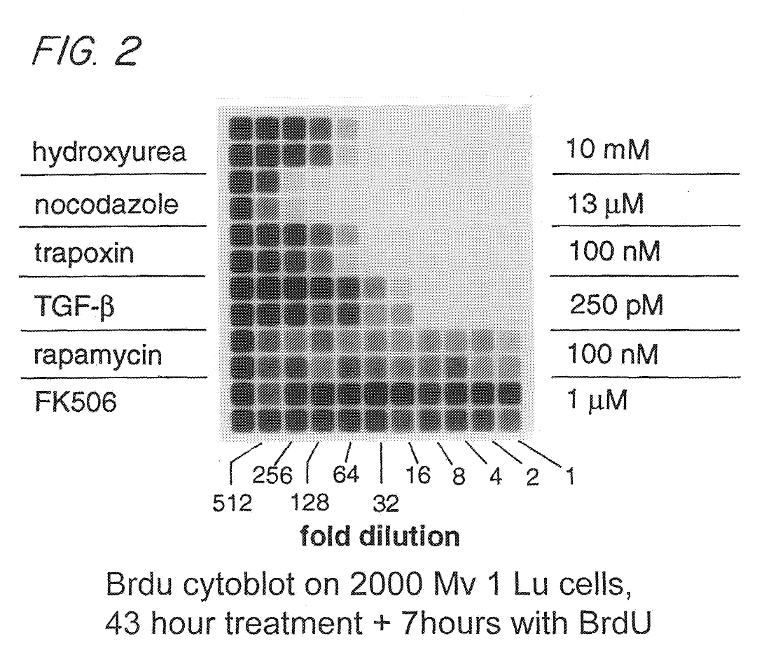

Small molecule antiproliferative agents that are capable of arresting the cell-cycle are tested for their ability to inhibit BrdU incorporation in the cytoblot assay. The results demonstrate that rapamycin, hydroxyurea, nocodazole, and trapoxin, effectively prevent BrdU incorporation (FIG. 2). In addition, the cytoblot assay demonstrates that FK506 fails to prevent BrdU incorporation (FIG. 2). Thus, these results show that the cytoblot assay was capable of discriminating between compounds that affect or that do not affect DNA synthesis.

### Example 2

### 1536 Well BrdU Cytoblot

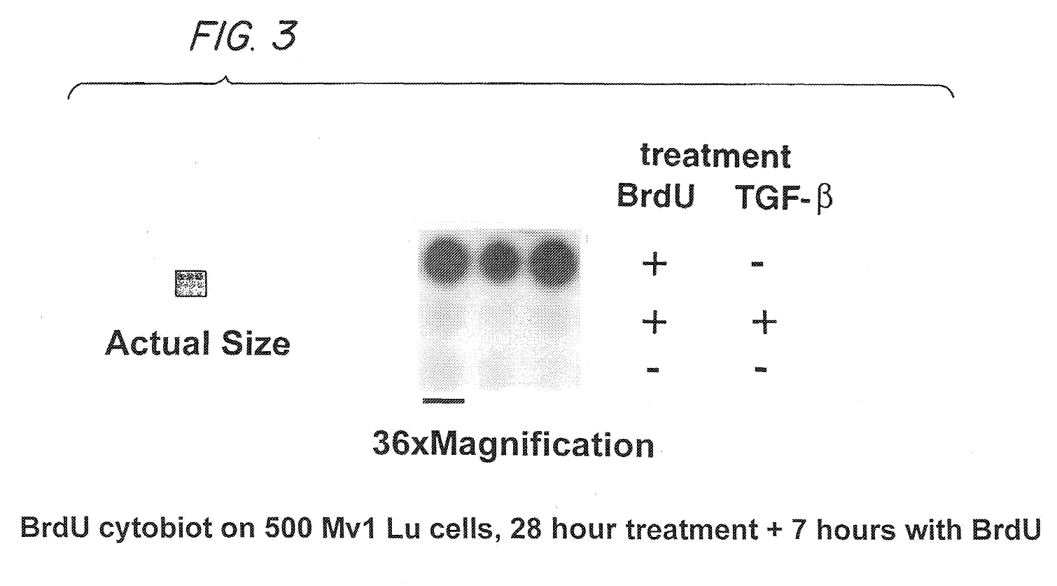

The number of compounds that the cytoblot assay can screen is greatly enhanced by increasing the samples per plate. Thus, the application of the cytoblot assay to plates containing a higher density of wells was tested. Mink lung cells were seeded into opaque, white 1536-well plates with each well containing approximately 500 cells. Next, the ability of TGF-β to prevent BrdU incorporation was assayed. As with the 384-well plates, TGF-β effectively prevents BrdU incorporation with negligible background staining (FIG. 3). The following protocol provides experimental detail.

The reagents to be tested were prepared in 1536 well plates (Greiner America, Lake Mary, Fla.), with one or more test compounds per well. If the test compounds were attached on solid support (beads), then the beads were distributed into individual wells of 1536 well white plate in acetonitrile. The compounds were released by photolysis or chemical treatment with some or all of that acetonitrile solution transferred to a new 1536 well plate. The organic solvent was then evaporated off. The cells were then seeded (2 μL per well, 500 Mv1Lu mink lung epithelial cells (American Type Culture Collection, Rockville, Md., Cat. # CCL64)) on the residue of the compound in the presence or absence of a known biological agent (e.g. 200 μM TGF-β1, Sigma Corporation, Cat.# T-1654).

After 16 to 36 hours, 0.5 μL of 50 μM BrdU (Sigma Corporation, Cat.# B9285) was added to each well for a final concentration of 10 μM BrdU. The cells were incubated for an additional 4 to 16 hours.

The plates were cooled on ice for 15 minutes. The plates were kept under aluminum foil and light exposure was minimized for all of the remaining operations. The supernatant was removed from each well by tilting the plate sideways and shaking or by simply washing in the next solution. The entire plate was immersed in each of the solutions of the protocol.

The plate was immersed in a cold (4° C.) solution of 70% ethanol/30% phosphate buffered saline (PBS). The plates were incubated one hour on ice. The ethanol/PBS solution was aspirated off and cold (4° C.) PBS was added. Next, the PBS solution was aspirated off and 2 M HCl/0.5% Tween 20/H₂O was added. The plates were incubated at room temperature for 20 minutes. The acid was aspirated off and a solution of 10% 2M NaOH/90% Hank's Balanced Salt Solution (HBSS, GibcoBRL, Cat. # 24020-117) was added. The base solution was aspirated off and HBSS was added. The HBSS was aspirated off and additional HBSS was added to each well. The HBSS was aspirated off and PBSTB (PBS; 0.1% Tween 20 (Sigma Corporation, Cat.# P-1379), 0.5% bovine serum albumin (Sigma Corporation, Cat.# A-2153)) was added to each well.

The PBSTB was removed and antibody solution was added. Antibody solution contained 0.5 μg/mL mouse anti-BrdU antibody (1:1000 dilution of stock, Pharmingen, San Diego, Calif., Cat.# 33281A) and a 1:2000 dilution of an anti-mouse Ig antibody conjugated to HRP (Amersham Corporation, Cat.# NA9310) in PBSTB. The plates were incubated for one hour at room temperature. The antibody solution was aspirated off and PBS was added. The PBS solution was aspirated off and more PBS was added to each well. The PBS solution was aspirated off and HRP substrate solution was added. The HRP substrate solution was obtained by mixing equal volumes of solutions 1 and 2 from the Amersham ECL detection kit (Cat.# RPN2106).

The plate was allowed to incubate for five minutes at room temperature. Then the plate was placed on a flat surface in a dark room. Some saran wrap may be placed on top of the plate to prevent contact between the substrate solution and the film. A piece of film (X-OMAT AR, Kodak Corporation, Rochester, N.Y.) was placed on top of the plate. Exposures of one minute and 5 minutes were usually sufficient for detecting BrdU activity in mink lung cells. Longer or shorter exposures can be made. The film was developed in a Kodak M35A X-OMAT processor (Kodak Corporation, Rochester, N.Y.).

### Example 3

### 6144 Well BrdU Cytoblot

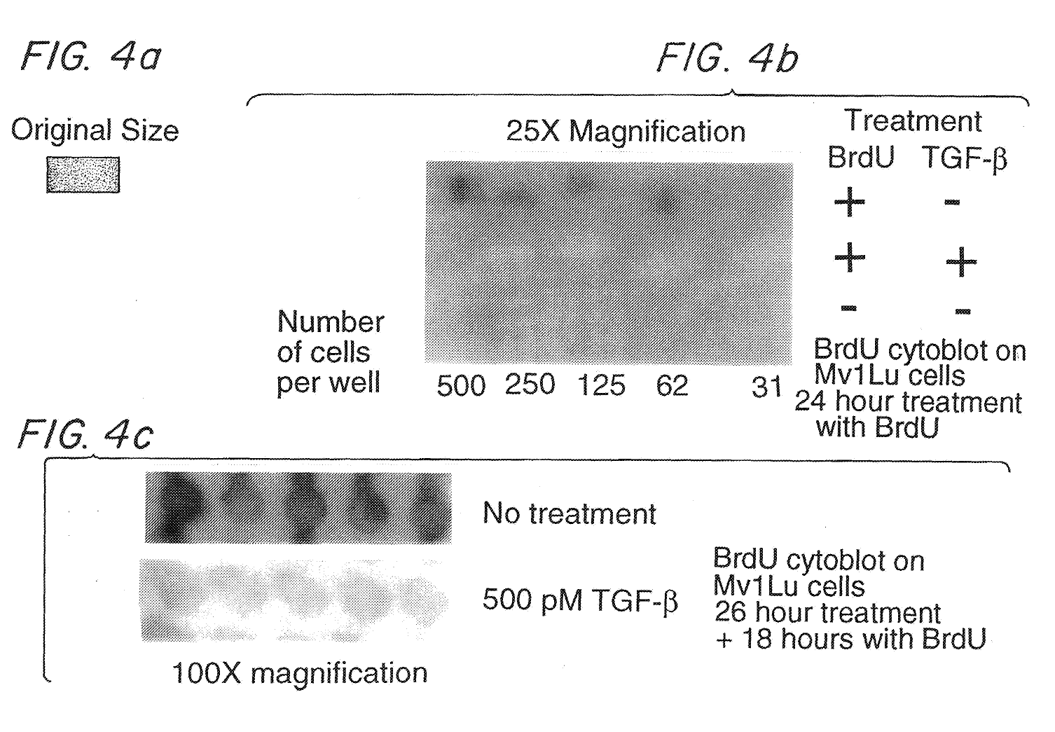

The anti-BrdU cytoblot assay was then tested for its ability to detect the inhibition of BrdU uptake in a plate containing approximately 6000 arrayed “nanowells” (You et al. Chem Biol 4:969-975. 1997. Incorporated herein by reference). Again TGF-β prevented BrdU incorporation with negligible background staining (FIG. 4). Importantly, individual wells were easily resolved (FIG. 4c), indicating that interwell contamination was not problematic. The following protocol provides experimental detail.

The reagents to be tested were prepared in 1536 well plates with one or more test compound per well. If the test compounds were attached on a solid support (beads), then the beads were distributed into individual wells of a 1536 well white plate in acetonitrile with the compounds released by photolysis or chemical treatment. Then, some or all of the acetonitrile solution containing the test compounds was transferred to a 6144 well plate and the organic solvent was evaporated off. The cells were then seeded (0.25 μL per well, 100 Mv1Lu (ATCC cat #CCL64) mink lung epithelial cells) on the residue of the compound in the presence or absence of a known biological agent (e.g. 200 μM TGF-β1, Sigma cat# T-1654) and 10 μM BrdU (Sigma cat# B9285).

After 24 hours, the plates were cooled on ice for 15 minutes. The plates were kept under aluminum foil and light exposure was minimized for all of the remaining operations. The supernatant was removed from each well by tilting the plate sideways and shaking or by simply washing in the next solution. The entire plate was immersed in each of the solutions of the protocol. The plate was immersed in a cold (4° C.) solution of 70% ethanol/30% phosphate buffered saline (PBS). The plates were incubated one hour on ice. The ethanol/PBS solution was aspirated off and cold (4° C.) PBS was added. The PBS solution was aspirated off and 2 M HCl/0.5% Tween 20/H₂O was added. The plates were incubated at room temperature for 20 minutes. The acid was aspirated off and a solution of 10% 2M NaOH/90% Hank's Balanced Salt Solution (HBSS, GibcoBRL, Cat.# 24020-117) was added. The base solution was aspirated off and HBSS was added. The HBSS was aspirated off and additional HBSS was added to each well. The HBSS was aspirated off and PBSTB was added to each well (PBSTB=0.1% Tween 20 (Sigma cat# P-1379)/0.5% Bovine albumin (Sigma Cat # A-2153)/PBS)

The PBSTB was removed and antibody solution was added. Antibody solution contains 0.5 μg/mL mouse anti-BrdU antibody (1:1000 dilution of stock, Pharmingen, cat #33281A) and a 1:2000 dilution of an anti-mouse Ig antibody conjugated to HRP (Amersham, cat#NA9310) in PBSTB. The plates were incubated for one hour at room temperature. The antibody solution was aspirated off and PBS was added. The PBS solution was aspirated off and more PBS was added. The PBS solution was aspirated off and HRP substrate solution was added. The HRP substrate solution was obtained by mixing equal volumes of solutions 1 and 2 from the Amersham ECL detection kit (Cat.# RPN₂₁₀₆).

The plate was allowed to incubate for five minutes at room temperature. Then the plate was placed on a flat surface in a dark room. Some saran wrap may be placed on top of the plate to prevent contact between the substrate solution and the film. A piece of film (Kodak X-OMAT AR) was placed on top of the plate. Exposures of one minute and 5 minutes were usually sufficient for detecting BrdU activity in mink lung cells. Longer or shorter exposures can be made. The film was developed in a Kodak M35A X-OMAT processor.

### Example 4

### 384 Well Anti-Acetylated H4 and Anti-Phospho Histone H₃Cytoblot

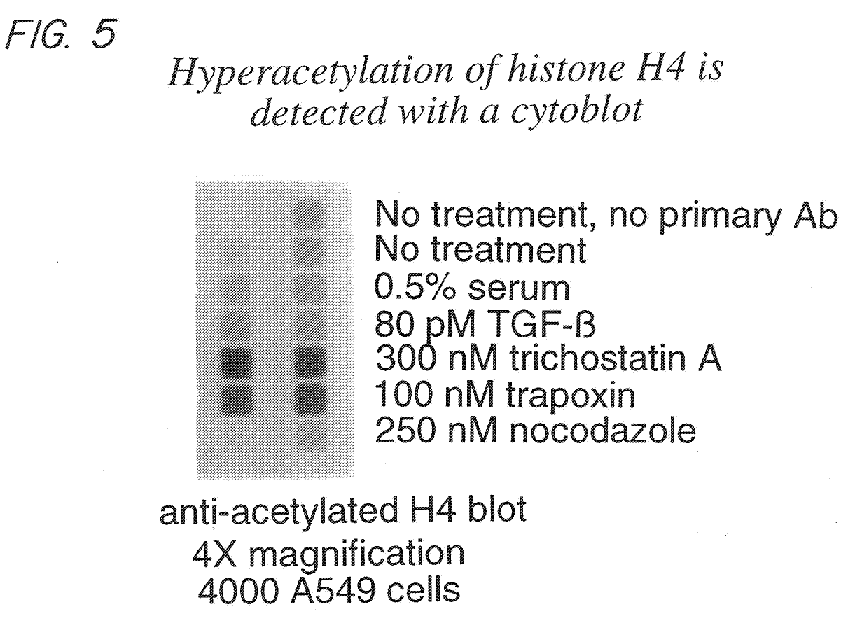

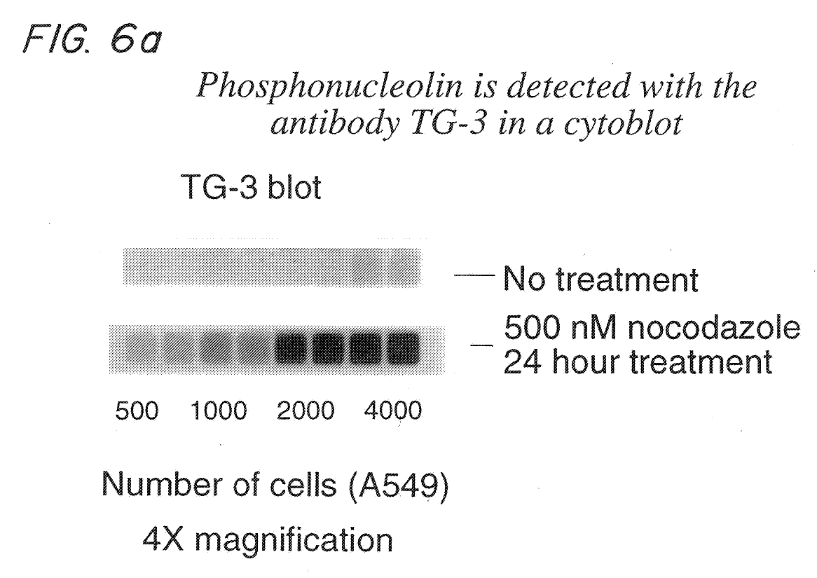

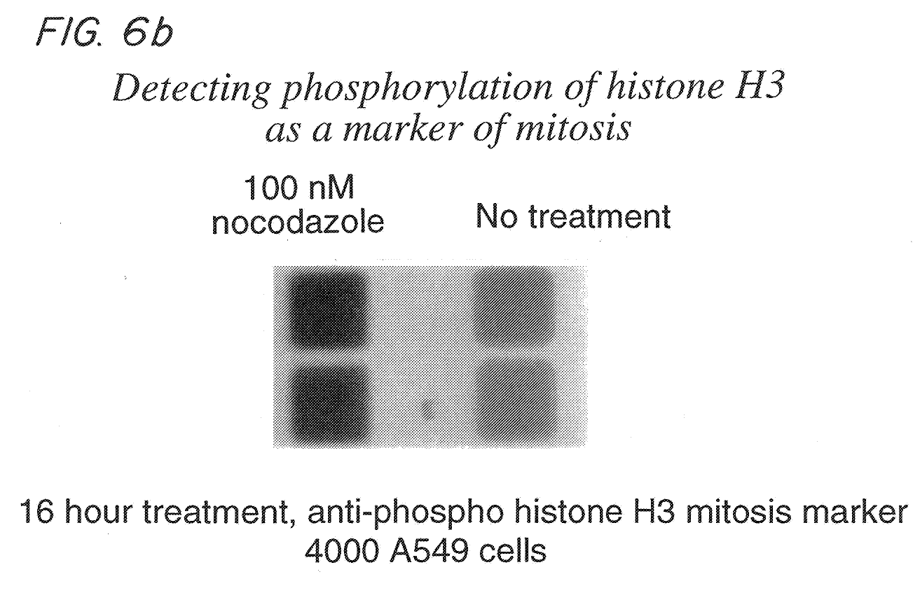

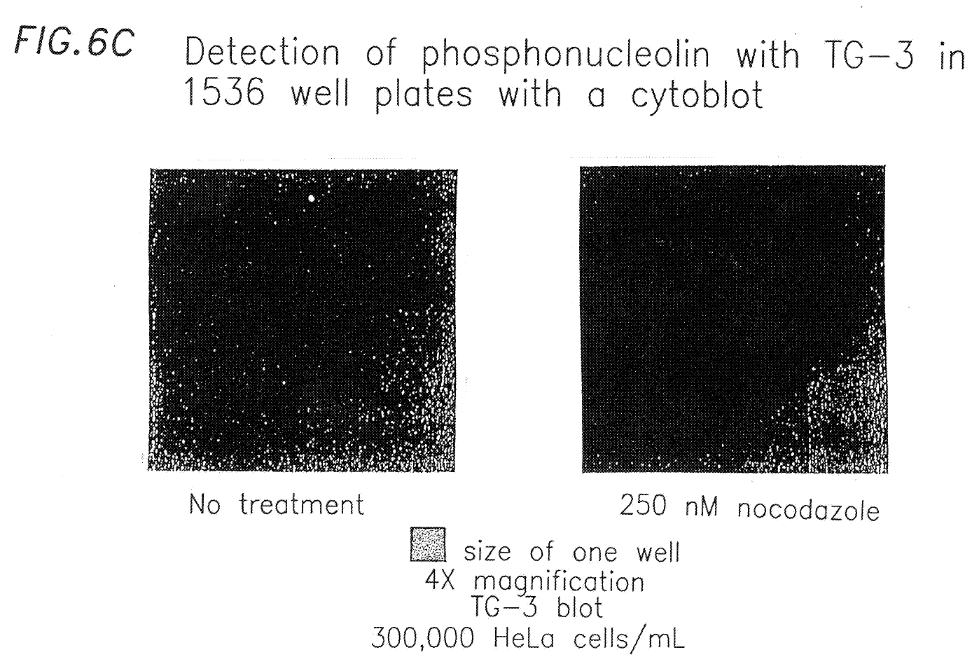

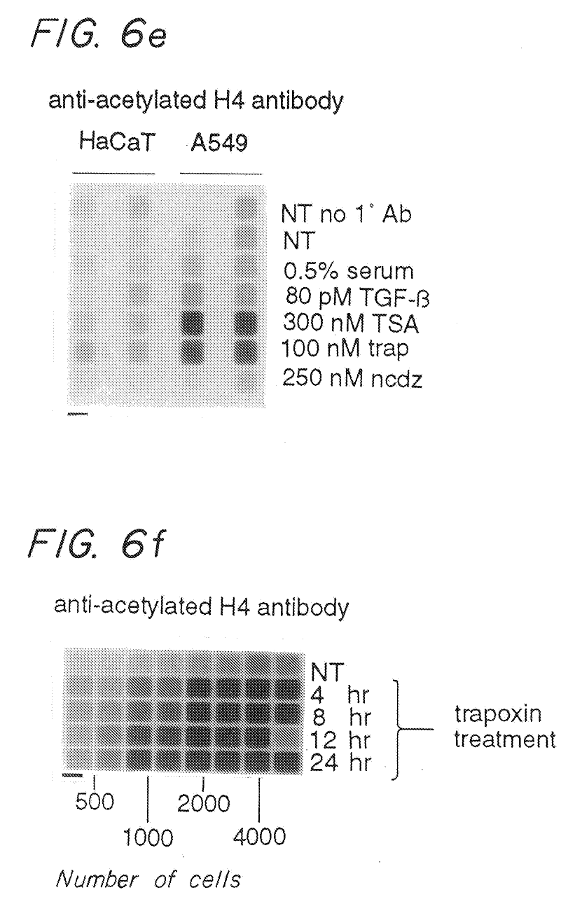

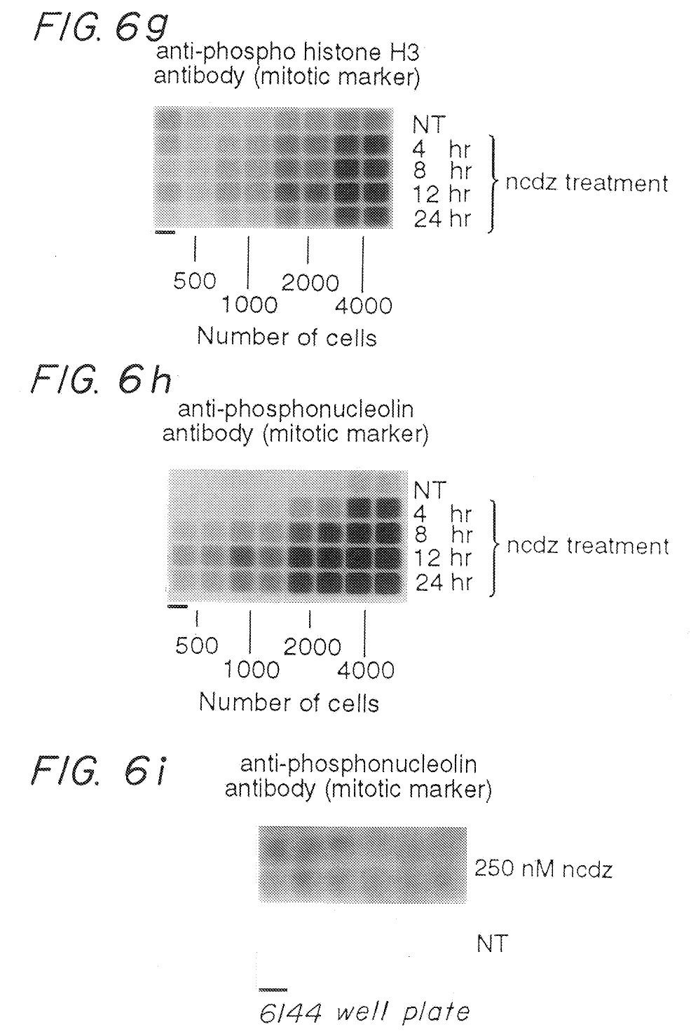

Any post-translational modification that can be recognized by an antibody (or ligand) may be used in a cytoblot screen. To demonstrate the feasibility of this application of the cytoblot assay, an anti-acetylated histone H4 antibody was used to detect an increase in the acetylation of histone H4 in the presence of the histone deacetylase inhibitors trapoxin A and trichostatin (FIG. 5). In addition, antibodies against the phosphorylated form of nucleolin (FIG. 6a) or histone H3 (FIG. 6b) were used to detect the presence of phosphonucleolin or phosphohistone H3 respectively, both of which server as biochemical markers of the mitotic state of cells (Anderson et al. Experimental Cell Research 238: 498-502 1998; Vincent et al. J. Cell. Biol. 132:413-425. 1996)). The following protocol provides experimental detail.

The reagents to be tested were prepared in 384 well plates with one or more test compound per well. If the test compounds were attached on solid supports (beads), then the beads were distributed into individual wells of a clear-bottom 384 well white plate (Costar cat# 3707) with a Multidrop 384 plate filler (Lab Systems) in acetonitrile. The compounds were released by photolysis or chemical treatment with some or all of the acetonitrile solution containing the test compounds transferred to a new 384 well white-bottom plate (Nalge Nunc International cat# 164610). The organic solvent was then evaporated off. Alternatively, if a concentrated stock solution was available, the compound was transferred into the test plate after the cells were seeded using small pins, syringes or pipettes to deliver approximately 50-500 mL. Cells were seeded in 40-45 μL at the indicated cell density (typically 4000 cells/well), allowed to attach overnight (12-14 hours) and then a known biological agent (e.g. trapoxin, 100 nM in DMSO for anti-acetylated histone H4 or 250 nM to 500 nM nocodazole for anti-phosphonucleolin) was added.

After 4 to 24 hours, the plates were cooled on ice for 5 minutes. The supernatant was removed from each well with a 24-channel wand attached to a vacuum source. 50 μL of cold (4° C.) Tris-buffered saline (TBS, 10 mM Tris, pH 7.4, 0.15 M NaCl) was added to each well. The TBS was aspirated off and 40 μL of a cold (4° C.) fixing solution of 3.7% formaldehyde in TBS was added to each well. The plates were incubated one hour at 4° C. The fixing solution was aspirated off and 30 μL of cold (−20° C.) 100% methanol was added to each well. The plates were incubated at 4% C for 5 minutes. The methanol was aspirated off and each well was washed with 90 μL of 3% milk in TBS, then 25 μL of an antibody solution was added. Antibody solution contained appropriately either: 1:100 dilution of anti-acetylated H4 antibody (Upstate Biotechnology, Lake Placid, N.Y. cat #06599) and 1:1000 dilution of anti-rabbit IgG antibody conjugated to HRP in 3% milk/TBS, a 1:100 dilution of anti-phospho Histone H3 Mitosis Marker antibody (Upstate Biotechnology, Lake Placid, N.Y., Cat. # 06-570) and 1:500 dilution of anti-rabbit IgG antibody conjugated to HRP in 3% milk/TBS, or a 1:250 dilution of TG-3 monoclonal supernatant and 1:7500 dilution of anti-mouse IgM antibody conjugated to HRP. The plates were incubated for 2-24 hours at 4° C. The antibody solution was aspirated off and the plates were washed twice with 90 μL of TBS. 30 μL HRP substrate solution was added to each well. The plates were allowed to incubate for five minutes at room temperature. Then the plate was placed on a flat surface in a dark room. A piece of film (Kodak X-OMAT AR) was placed on top of the plate. Exposures of five to ten minutes were sufficient for detecting hyperacetylation of histone H4 in A549 cells and one to three minutes were sufficient for detecting phosphorylation of histone H3 or nucleolin.

### Example 5

### 384 well TG-3 Mitotic Antibody Cytoblot

The reagents to be tested were prepared in 384 well plates, one or more test compound per well. If the test compounds were on solid support (beads), then the beads can be distributed into individual wells of a clear-bottom 384 well white plate (Costar cat# 3707) with a Multidrop 384 plate filler (Lab Systems) in acetonitrile, and compound was released by photolysis or chemical treatment, and then some or all of that acetonitrile solution was transferred to a new 384 well white-bottom plate (Nalge Nunc International cat# 164610) and the organic solvent was evaporated off. Alternatively, if a concentrated stock solution was available, the compound was transferred into the test plate after the cells were seeded using small pins, syringes or pipettes to deliver approximately 50-500 nL. The cells were seeded (40 μL per well, 2000 HeLa cells), allowed to attach overnight and a known biological agent (e.g. 133 nM nocodazole, Sigma cat# M1404) was added.

After 4 to 24 hours, the plates were cooled on ice for 5 minutes. The supernatant was removed from each well with a 24 channel wand (V&P Scientific) attached to a vacuum source. This wand was used for aspiration throughout the protocol. 50 μL of cold (4° C.) Tris-buffered saline (TBS, 10 mM Tris, pH 7.4, 0.15 M NaCl) was added to each well. The TBS was then aspirated off. 50 μL of a cold (4° C.) fixing solution of 3.7% formaldehyde was added to each well. The plates were incubated one hour on ice. The fixing solution was aspirated off and 30 μL of cold (−20° C.) 100% methanol was added to each well. The plates were incubated at 4° C. for 5 minutes. The methanol was aspirated off and 90 μL of 3% milk (BioRad)/TBS was added to each well. The milk solution was aspirated off and 20 μL of a antibody solution was added.

Antibody solution contains 1:250 dilution of TG3 antibody and 1:7500 dilution of anti-mouse IgM antibody conjugated to HRP (Amersham, cat# NA9310) in 3% milk (BioRad)/TBS. The plates were incubated for 2-24 hours at 4° C. The antibody solution was aspirated off and 90 μL of TBS was added to each well. The TBS was aspirated off and an additional 90 μL of TBS was added to each well. The TBS solution was aspirated off and 30 μL HRP substrate solution was added to each well. The HRP substrate solution was obtained by mixing equal volumes of solutions 1 and 2 from the Amersham ECL detection kit (cat#RPN2106).

The plate was allowed to incubate for five minutes at room temperature. Then the plate was placed on a flat surface in a dark room. A piece of film (Kodak X-OMAT AR) was placed on top of the plate. Exposures of one minute and 5 minutes were usually sufficient for detecting phosphonucleolin in HeLa cells. Longer or shorter exposures can be made. The film was developed in a Kodak M35A X-OMAT processor.

### Example 6

### Small Molecule Suppressors of Antiproliferative Agents

To demonstrate the ability of the cytoblot assay to detect small molecule suppressors of antiproliferative agents, the immunosuppressive natural product FK506 was assayed for its ability to act as a suppressor of rapamycin. This particular experiment relies on the fact that FK506 and rapamycin share a common binding protein, FKBP12 (FK506 and rapamycin binding protein, 12 kilodaltons). As a heterodimeric complex, rapamycin/FKBP12 binds to the protein, FRAP (FKBP12-rapamycin associated protein). Alternatively when FKBP12 is complexed with FK506, this heterodimer binds to the phosphatase calcineurin. Since the antiproliferative effect of rapamycin is dependent on the formation of the rapamycin/FKBP12 complex, excess FK506 could potentially prevent rapamycin-induced growth arrest by titrating away FKBP12, thus preventing the formation of the rapamycin/FKBP12 complex.

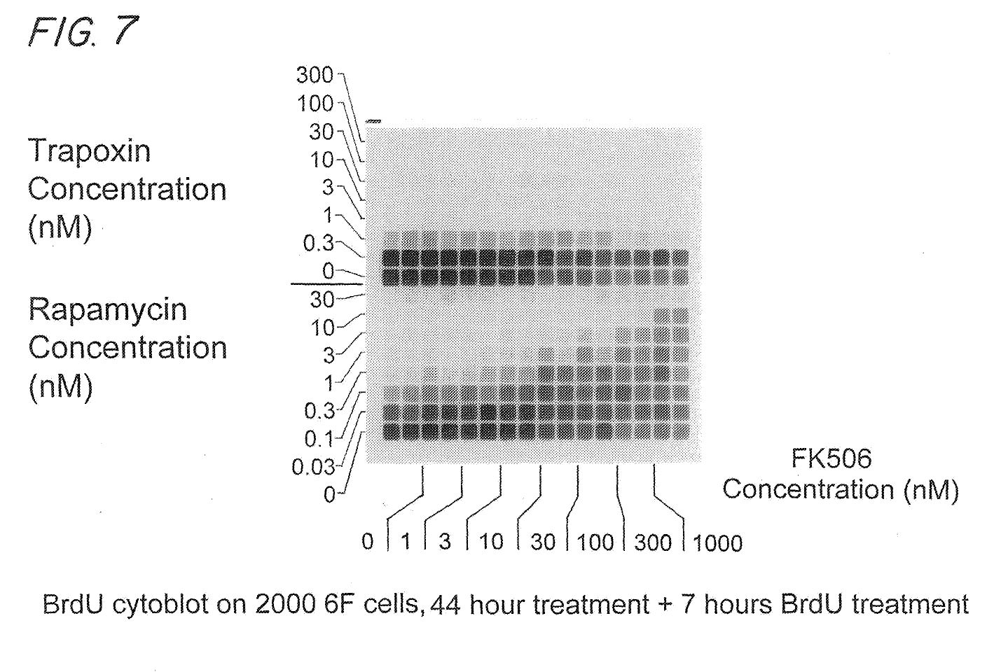

This suppression of the ability of rapamycin to inhibit growth is demonstrated in the cytoblot assay by the simultaneous treatment of mink lung cells with rapamycin and excess FK506. The excess FK506 should result in the ability of cells to incorporate BrdU in the presence of rapamycin. Mink lung cells were seeded into 384-well plates and treated with varying concentrations of both rapamycin and FK506 together for 16 hours and then treated with rapamycin, FK506 and BrdU for 16 hours. FIG. 7 shows that a 30-100 fold excess of FK506 suppresses the antiproliferative effect of rapamycin. Thus, the cytoblot assay is capable of detecting small molecule suppressors of antiproliferative agents. This suppressor screening strategy can be applied to other antiproliferative agents, including but not limited to ones such as TGF-β, hydroxyurea, nocodazole, mimosine, benomyl, trapoxin, trichostatin and depudicin.

### Example 7

### A Screen for Natural Products Suppressors of Anti-proliferative Agents

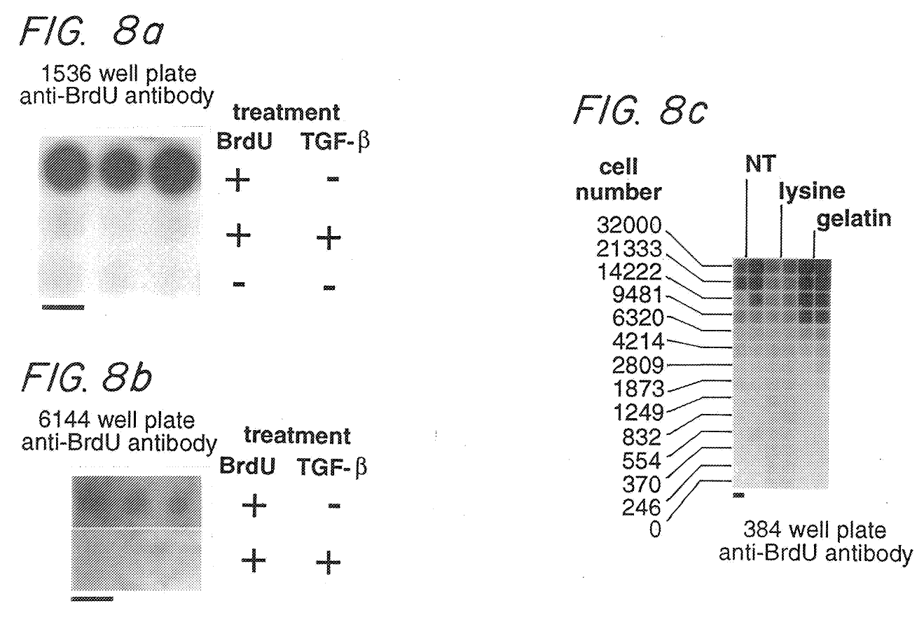

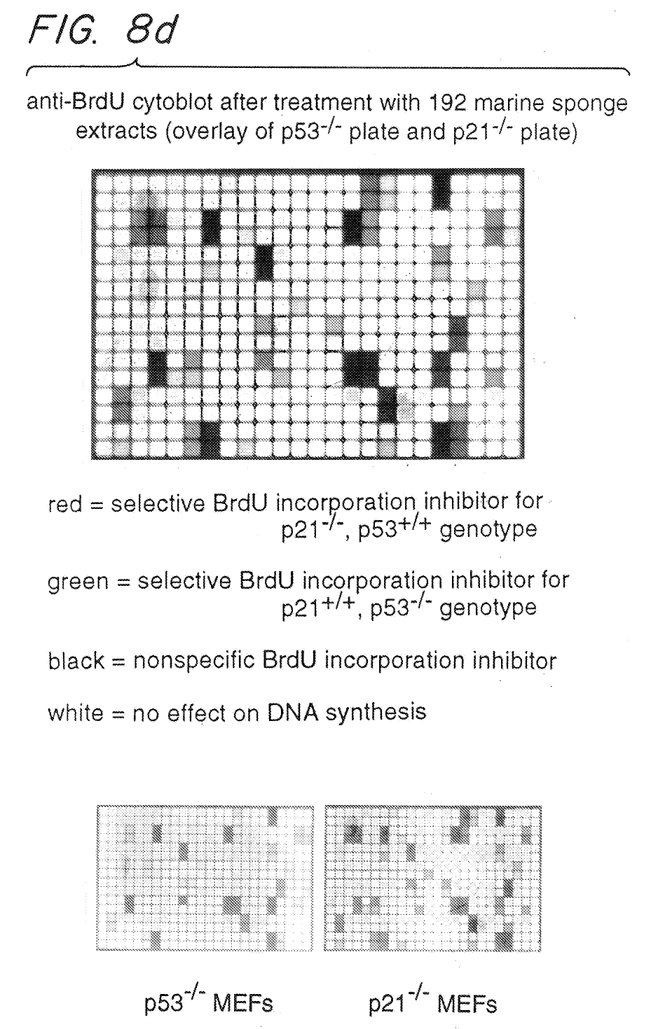

Although general inhibitors of DNA synthesis may be useful and interesting compounds, genotype-specific inhibitors of DNA synthesis may also be useful. We obtained 192 marine sponge extracts from Professor Phillip Crews and Miranda Sanders and we tested these crude organic extracts (in duplicate rows) for their ability to inhibit BrdU incorporation in either p53^{-/-} or p21^{Kip1-/-} mouse embryonic fibroblasts (MEFs) using a BrdU cytoblot (FIG. 8d). By overlaying the results of these experiments, we were able to identify extracts that were genotype-independent BrdU incorporation inhibitors (black wells), p21^{Kip1-/-}p53^{+/+}-specific BrdU incorporation inhibitors (red wells), and possibly some weak p21^{+/+}p53^{-/-} specific BrdU incorporation inhibitors (green wells). It should be noted that loss of BrdU incorporation in this assay may be due to either cytostatic or cytotoxic effects and further characterization of these extracts will be required to distinguish these two effects.

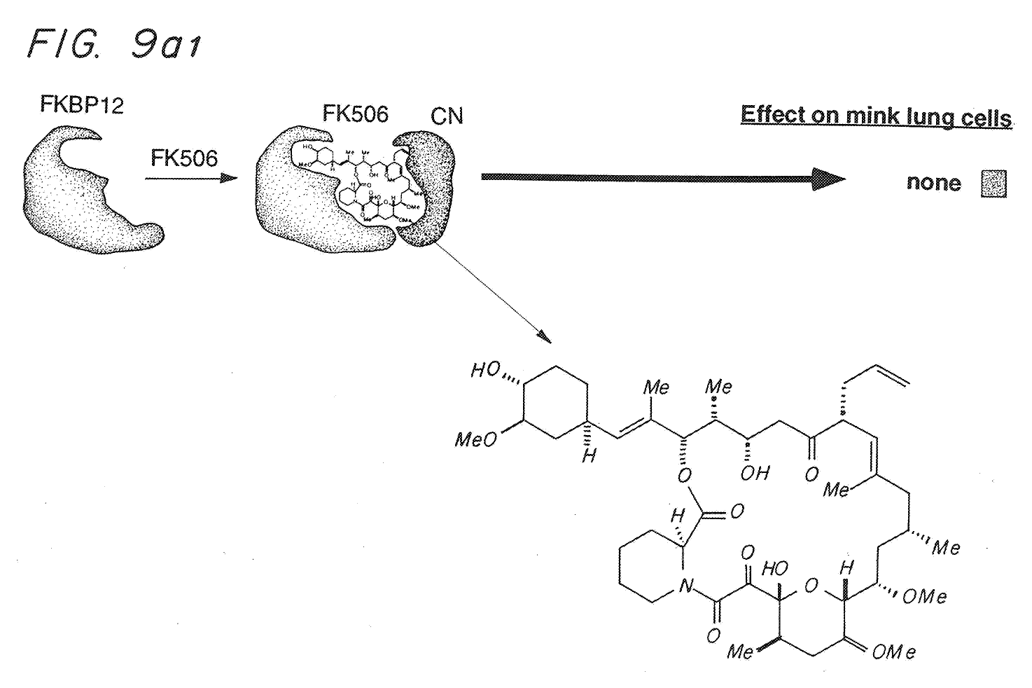

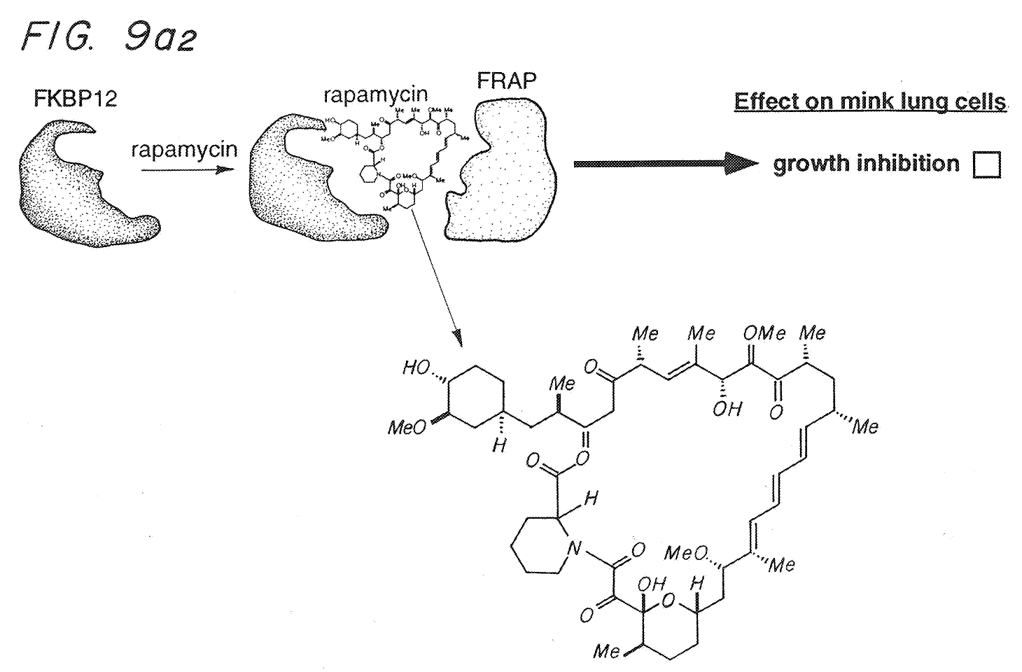

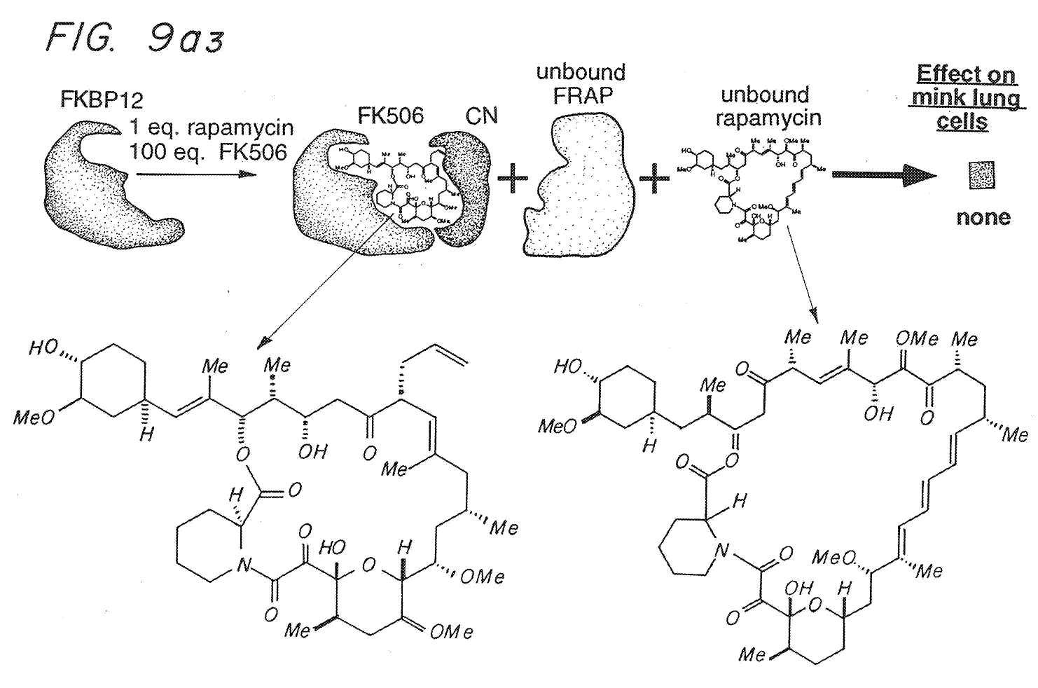

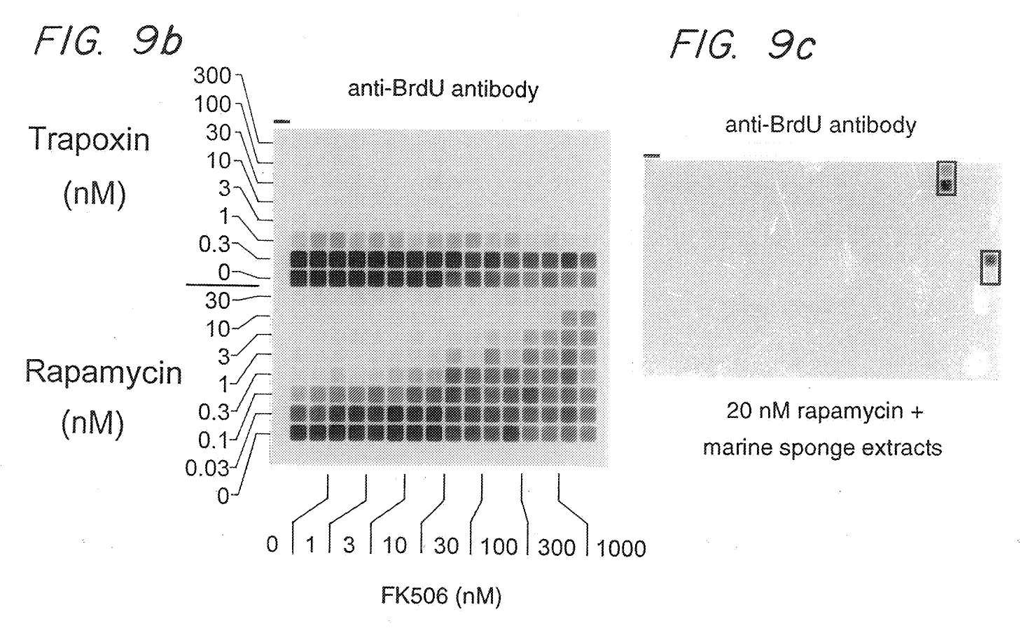

To demonstrate that it is possible to identify natural products that act as suppressors of anti-proliferative agents, we screened 192 marine sponge extracts, kindly provided by Professor Phillip Crews and Miranda Sanders (UCSC), for suppressors of the anti-proliferative effect of rapamycin. We identified two crude organic extracts that allowed mink lung cells to incorporate BrdU in the presence of 20 nM rapamycin (FIG. 9c), a concentration that otherwise prevents BrdU incorporation in these cells (FIG. 9b). The extracts were tested in duplicate rows and the two hits are shown in red boxes (FIG. 9c). A third extract with weak suppressor activity was visible upon longer exposure to film (data not shown). All three hits were confirmed by retesting the extracts in duplicate. These active extracts were generated from Indo-Pacific marine sponges, collected by the Crews group. Two of these samples came from sponges in the family Petrosiidae, and the third originates from a specimen most closely resembling Callyspongia ramosa. All three sponges belong to the order Haplosclerida. Taxonomic identification of the source organisms and further chemical analysis of the active extracts are now underway. In collaboration with Crews and Sanders, we hope to test further these extracts in this suppressor assay. This suppressor screening strategy can also be applied to other anti-proliferative or cytostatic proteins and small molecules such as TGF-β, hydroxyurea, mimosine, lovastatin, nocodazole, benomyl, and depudicin, as well as DNA-damaging agents such as mitomycin, bleomycin, cisplatin, ultraviolet light and gamma irradiation

### Example 9

### Assaying Small Molecule Suppressors of Cell-Cycle Arresting Agents

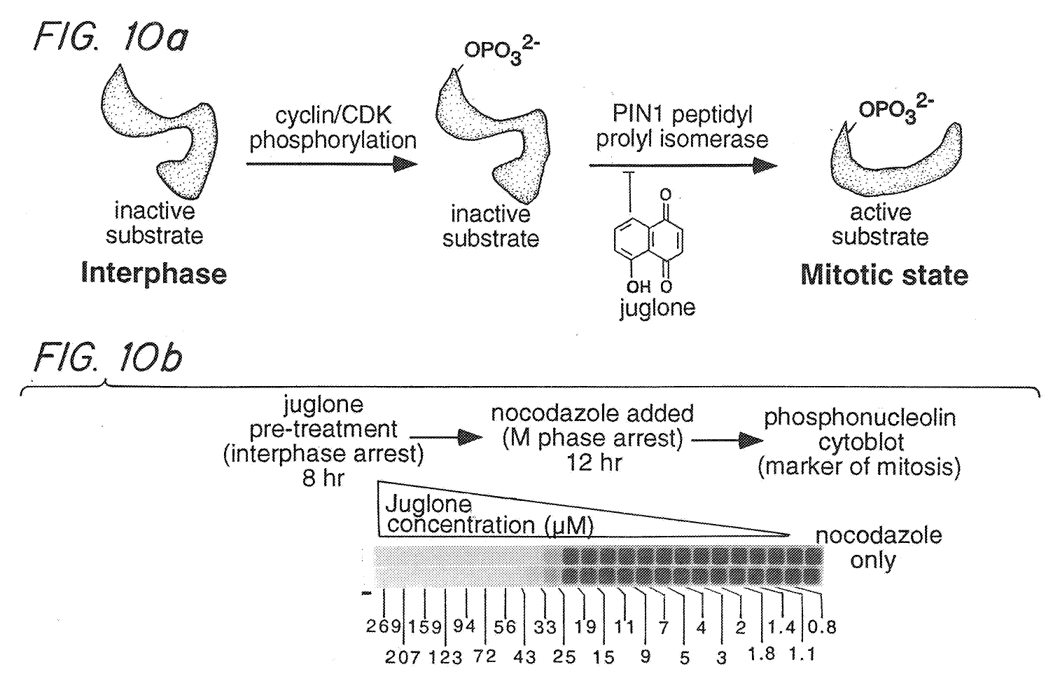

The treatment of cells with any small molecule that arrests cells outside of mitosis will suppress the ability of nocodazole to arrest cells in mitosis. Recently the natural product juglone was demonstrated in vitro to be a selective, covalent inhibitor of Pin1, a member of the parvulin family of peptidyl-prolyl cis/trans isomerases (PPIases). It was hypothesized that inhibition of Pin1, which is an essential, highly conserved PPIase required for proper mitotic division in Xenopus and yeast, would prevent entry into mitosis and consequently prevent accumulation of phosphonucleolin (FIG. 10a). We found this to be the case at concentrations greater than 25 microM juglone (FIG. 10b). Similar results were obtained using trapoxin and camptothecin (data not shown), both of which arrest cells outside of M-phase.

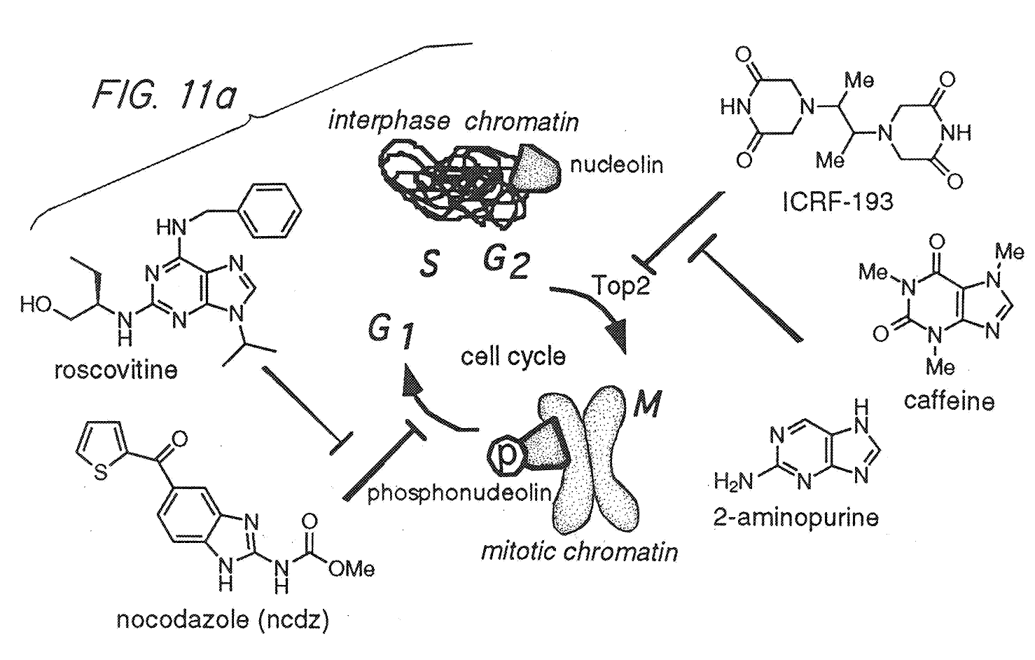

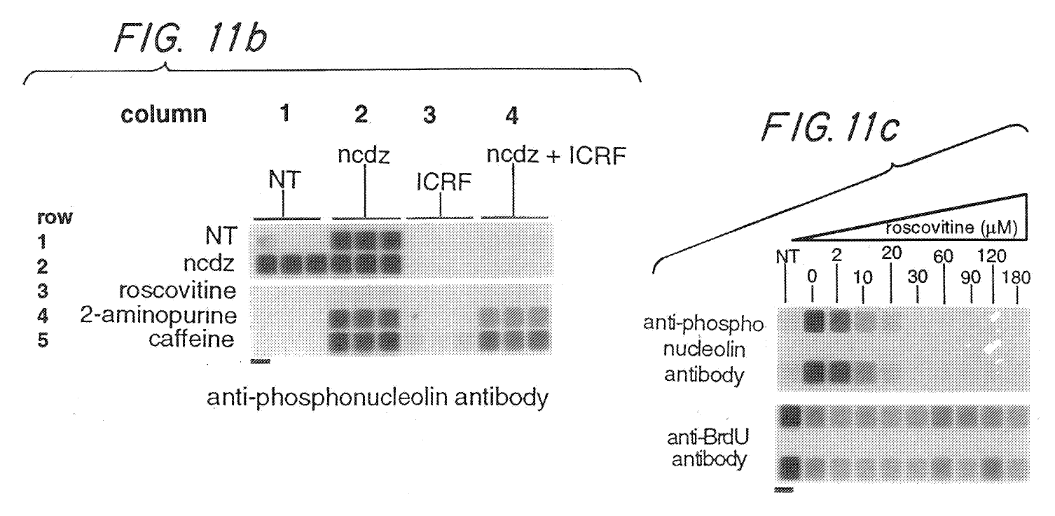

The juglone experiment (FIG. 10b) demonstrates that it is possible to find molecules that suppress the effects of nocodazole treatment by preventing entry into mitosis. It should also be possible to find small molecules that induce exit from mitosis, resulting in a reduction in phosphonucleolin levels. As the nocodazole-induced arrest of cells in mitosis requires cyclin-dependent kinase (CDK) activity for the activation of the spindle assembly checkpoint, inhibition of CDK activity should suppress nocodazole-induced mitotic arrest (FIG. 11a). We pretreated cells with nocodazole for 14 hours to arrest cells in mitosis (resulting in the accumulation of phosphonucleolin) and then added increasing amounts of roscovitine, a small molecule inhibitor of CDKs, for 8 hours. As a result of roscovitine treatment, cells exited from the nocodazole-induced arrest, as measured by the disappearance of phosphonucleolin (FIG. 11c) and flow cytometry (data not shown). However, based on a similar anti-BrdU cytoblot (FIG. 11c), these cells did not begin to incorporate BrdU, indicating that the ability to proliferate was not restored by roscovitine treatment. This is not surprising, since CDK activity is required for S-phase progression.

### Example 10

### Assaying Small Molecule Suppressors of G2-Arresting Agents

To demonstrate that it is possible to find small molecule suppressors of G₂-arresting agents (i.e., small molecules that allow entry into mitosis in the presence of a G₂-arresting agent), the ability of purine analogs to suppress the G₂ arrest caused by the topoisomerase II (Top2) inhibitor ICRF-193 (FIG. 11a) was tested. These purine analogs are known to be non-specific competitive inhibitors of 5′-adenosine triphosphate binding and therefore are likely to inhibit a wide range of kinases in the cell. As expected, cells treated with nocodazole arrested in mitosis and therefore contained substantial phosphonucleolin (FIG. 11b, row 1, column 2). The simultaneous addition of either ICRF-193 (row 2, column 3), or roscovitine (row 3, column 2), prevented this mitotic arrest, presumably by arresting the cells in interphase. The simultaneous addition of 2-aminopurine or caffeine, however, prevented the arrest in response to ICRF-193 and allowed cells to accumulate in mitosis in the presence of nocodazole (rows 4-5, column 4). As expected, nocodazole was required for the accumulation of cells in mitosis (rows 4-5, column 3). Thus, it is possible to screen for inhibitors of cell cycle arresting agents using both anti-BrdU and anti-phosphonucleolin antibodies in the cytoblot format.

### Example 11

### Use of Inventive Cytoblot to Identify Compounds that Alter Progression Through the Mammalian Cell Cycle

### Background

Understanding and controlling cell cycle regulation has important implications in modern day biology. Specifically, cell cycle inhibitors are important for use as anti-proliferative agents in the treatment of cancer and pathogenic infection, for preventing or reducing atherosclerosis or restenosis, as immunosuppressants, and for research purposes that require reagents capable of synchronizing the cell cycle of cell cultures or extracts to name a few. In addition, new cell cycle inhibitors may be vital to the elaboration and dissection of key regulatory steps of the cell cycle pathway. Thus, expansion of the number of useful compounds and reagents that can be used both therapeutically and for research related activities through the identification of new cell cycle inhibitors is highly desirable.

The cell cycle is controlled by a complex system of specifically timed events to ensure the integrity of the cell and its genetic material as the cell prepares to divide. It is well known that many small molecules inhibit progression of the cell cycle by binding to a protein or proteins required for cell division. The search for these small molecules has been an intensive area of investigation in recent decades. The majority of these compounds induce mitotic arrest by interfering with cytoskeletal organization or by affecting the microtubules required for spindle formation and chromosome segregation.

Microtubules are cellular structures present in all eukaryotic cells and play a key role in mitosis. Microtubules are also essential to other cellular activities, such as maintenance of cell shape, cell motility, cell anchorage, intracellular transport, cellular secretory activity, modulating the interactions of growth factors with cell surface receptors and intracellular signal transduction. Being filamentous in nature, microtubules are self-assembling and self-disassembling structures that are composed of the protein tubulin. Tubulin is itself a heterodimeric protein made up of α and β subunits. The cellular functions of microtubules rely on their being dynamic structures that undergo periods of slow growth and rapid shortening both in vitro and in cells (Mitchison and Kirschner, Nature, 1984, 312, 237-242; Schulze and Kirschner, J. Cell Biol., 1986, 102, 1020-1031; Cassimeris et al., J. Cell Biol., 1988, 107, 2223-2231).

A variety of antimitotic drugs interact with tubulin to alter the dynamic instability of microtubules (Hung et al., Chemistry& Biology, 1996, 3, 623-639; Jordan et al., Proc. Notal. Acad. Sci. U.S.A. 1993, 90, 9552-9556). Interference with the normal equilibrium between the microtubule and its subunits would be expected to disrupt cell division and motility as well as other cellular activities dependent on microtubules. This strategy has been successful in treating a wide variety of malignancies. For example, colchicine and the vinca alkaloids are among the most potent anticancer drugs. These antimicrotubule agents promote microtubule disassembly and play principal roles in the chemotherapy of most curable neoplasms including acute lymphocytic leukemia, Hodgkin's and non-Hodgkin's Lymphomas, and germ cell tumors, as well as the palliative treatment of many other cancers.

Another class of antimicrotubule agents act by promoting the formation of unusually stable microtubules by inhibiting the normal dynamic reorganization of the microtubule network required for mitosis and cell proliferation (Schiff et al., Nature, 1979, 277, 665; Schiff et al., Biochemistry, 1981, 20, 3247). Compounds that fall within this class of microtubule agents include taxol (Paclitaxel™), originally isolated from the stem bark of the western (Pacific) yew tree Taxus brevifolia, and epothilones A and β isolated from the bacterium Sorangium cellulosum. Taxol binds to tubulin and acts to stabilize cell microtubules and prevent their depolymerization (Horwitz et al., Nature, 1987, 277, 665-667). Thus, taxol increases the time required for cell division which in turn inhibits tumor activity. Taxol has been shown to have a very broad spectrum of activity against refractive ovarian cancer, metastatic breast cancer, head and neck cancer, malignant melanoma, as well as lung cancer (Bollag et al., Cancer Research, 1995, 55, 2325-2333). Epothilones A and B have minimal structural analogy to taxoids and stabilize microtubules in a similar manner to taxol. Like taxol, epothilones A and B are able to arrest cells in mitosis, cause formation of bundles of intracellular microtubules in non-mitotic cells, and induce the formation of hyperstable tubulin polymers.

Although taxol has been shown to be efficacious in the treatment of a number of solid tumors, its clinical success has been limited by its the side effects associated with its administration to human patients. Side effects include severe allergic reactions, neutropenia, peripheral neuropathy, and alopecia. In addition, taxol has a low solubility which complicates in vivo administration. Multiple drug resistance is another major limitation to the applicability of taxol to the treatment of human cancer. Taxol is a substrate for P-glycoprotein, a molecule that pumps cytotoxic compounds out of multiple drug resistant cells. In addition, synthesis of taxol in bulk is a is complicated procedure requiring time and expense. Lastly, the structure of taxol is complex and presents a major obstacle to facile chemical modification aimed at improving the molecules solubility and reducing associated side effects.

There exists a need to discover new and unique compounds that act as cell cycle inhibitors. Such compounds may prove to be useful for research purposes to identify key players in the cell cycle or may be new and useful treatments for cancer and other ailments. Such new and unique compounds may also have the added benefit of rapid and inexpensive synthesis. Alternatively or additionally the compound may be soluble and easily administered to a patient for treatment of an ailment requiring a cell cycle inhibitor. There also exists the need to discover compounds with microtubule inhibiting effects that elicit fewer side effects and retain a greater toxicity in multiple drug resistant cells. Discovery of a novel class of drugs that stabilize microtubules or interfere with the mitotic cytoskeleton to inhibit the cell cycle may lead to the development of more efficacious cancer chemotherapeutics and treatments for other related conditions with this same mechanism of action.

### Summary of Findings

We have used the inventive cytoblot system to identify compounds that alter the progression of mammalian cells through the cell division cycle. In particular, we have found one set of compounds that exhibit the vinblastine-like property of destabilizing microtubules, one set of compounds that exhibit the taxol-like property of stabilizing microtubules, and one set of compounds that alters chromosome segregation in a novel fashion. Of particular interest are the microtubule destabilizing compounds.

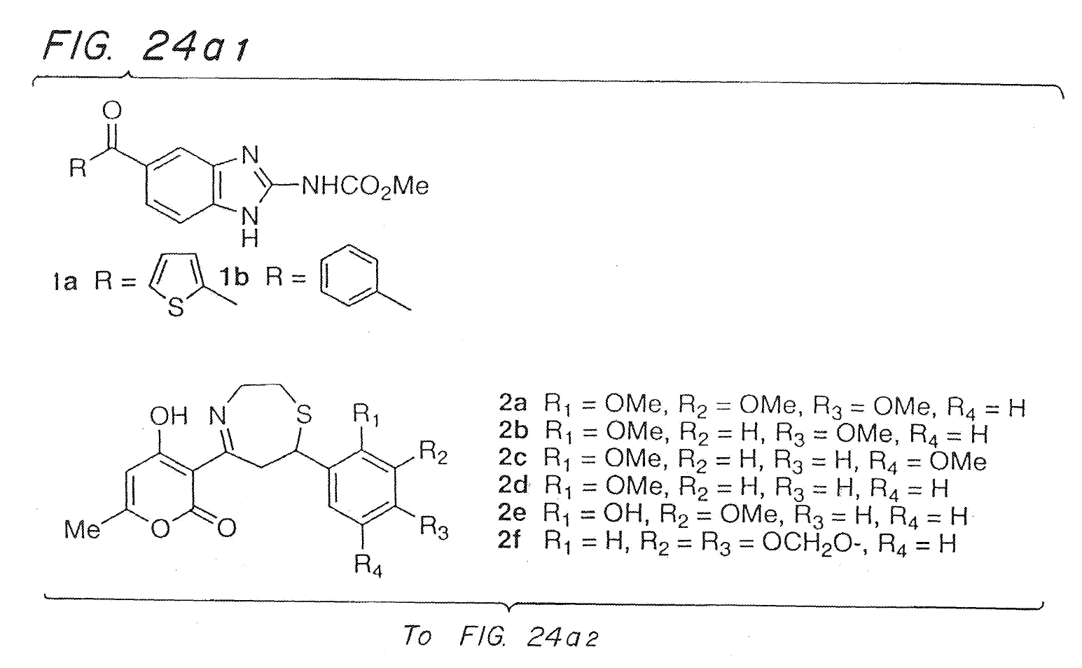

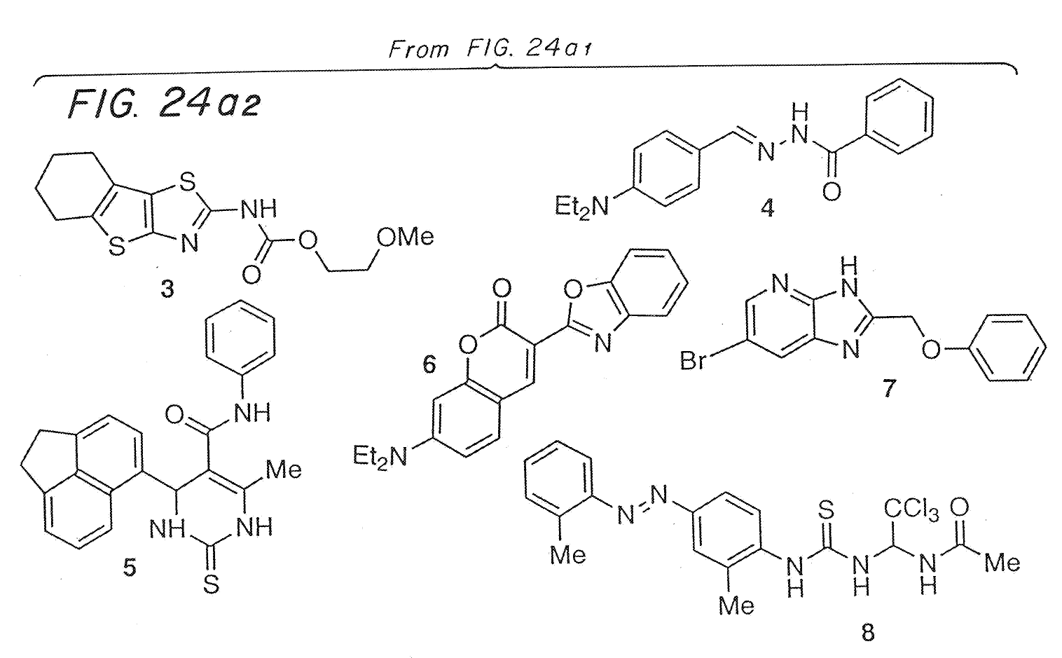

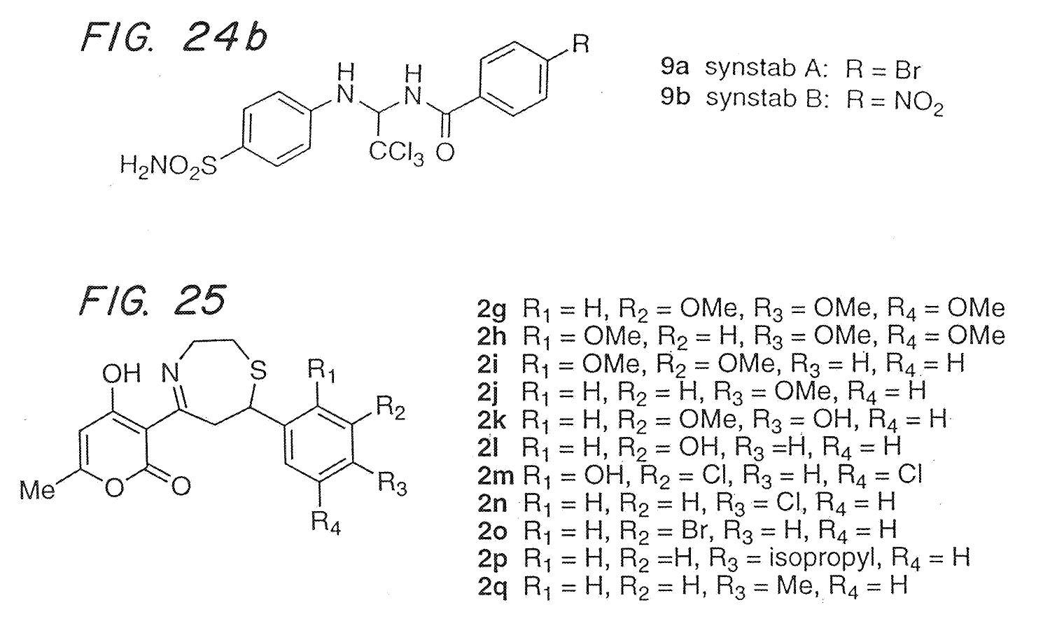

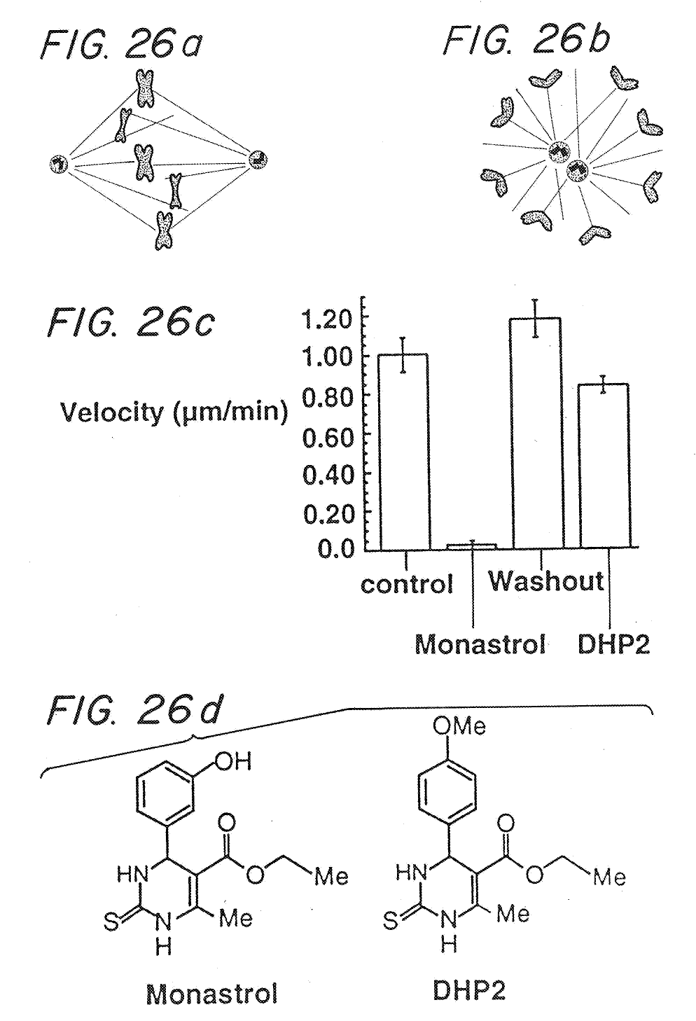

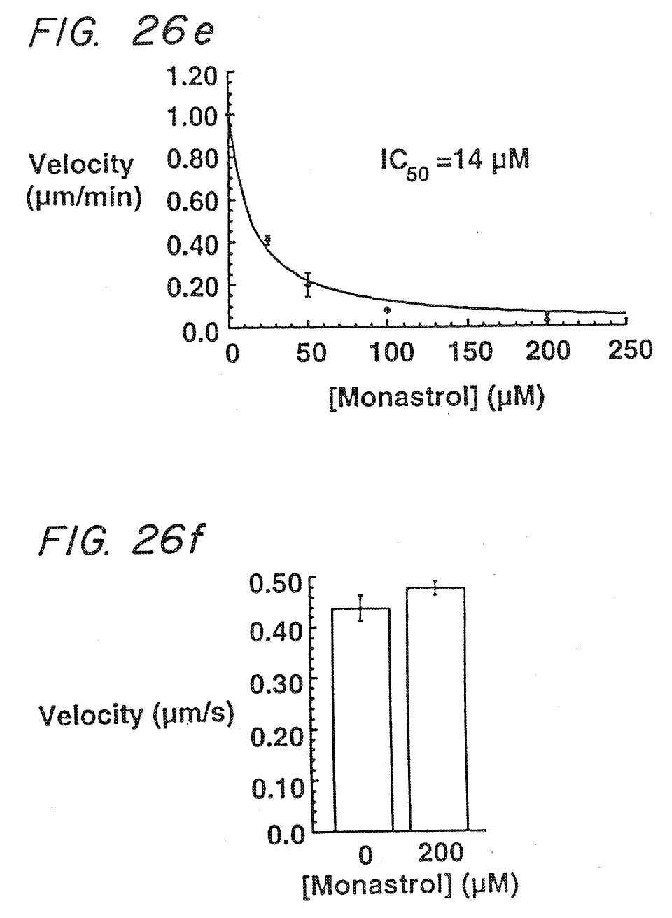

In general, the present invention provides compounds and pharmaceutical compositions that alter the progression of cells through the cell cycle (see FIGS. 24-26). Compounds of particular interest are summarized in FIG. 26 In certain preferred embodiments, the compounds and are capable of acting as inhibitors of the cell cycle. Specifically, these compounds are useful as microtubule stabilizers and as specific effectors of the cytoskeleton. In one aspect, the present invention provides novel compounds as shown by (10), (20), (30), (40), (50) and (60) below, and as described below. Furthermore, the present invention provides pharmaceutical compositions comprising a therapeutically effective amount of the compound having any one of the structures (10), (20), (30), (40), (50), or (60), associated with a pharmaceutically acceptable carrier.

As discussed, the present invention provides useful compositions comprising inhibitors of the cell cycle. Thus, in another aspect, the present invention provides methods of inhibiting cell cycle progression by 1) providing a system undergoing the cell cycle and 2) contacting the system with a chemical compound or composition having the general structures as disclosed herein.

In one preferred embodiment, the system undergoing the cell cycle is an in vivo system such as cells in culture. In another preferred embodiment, the system undergoing the cell cycle is an in vivo system in an organism. The inventive compounds and compositions can be used to treat a subject in need of anti-proliferative agents such as anti-cancer agents. More generally, the pharmaceutical compositions of the present invention may be administered to a subject in need of treatment with an agent that stabilizes microtubule polymerizations. Thus, the present invention also provides a method for treating a disorder comprising administering a therapeutically active composition of the present invention to a patient in need thereof.

In addition to their utility for pharmaceutical applications, the compounds of the present invention are also useful for basic scientific research purposes. For example, the compounds of the present invention that affect microtubule stability may be used to identify new cytoskeletal proteins and to unravel their regulation and function once identified. Additionally, the compounds of the present invention may be used in in vitro or in vivo mitotic assays to dissect the mitotic cycle.

It is well known that cell proliferation primarily requires 1) DNA replication and 2) cell division. Complete replication of the cell's genetic information must necessarily proceed chromosomal segregation and cell division in order to ensure the integrity of transmission of all genetic information. The cell cycle has four defined sequential phases: G1 is the first gap phase in which the cell prepares for DNA replication; S phase is the phase of DNA synthesis during which a complete copy of the entire genome is generated; G2 is the second gap phase in which the cell prepares for division; and lastly, M phase (mitosis) is the period in which the two copies of DNA segregate to two identical daughter cells during cell division.

Segregation of chromosomes to the daughter cells requires the activity of the spindle apparatus which attaches to and pulls apart the two identical sets of chromosomes. The spindle apparatus is composed of microtubules that are in dynamic equilibrium and are capable of complex reorganization to achieve cell division. A damaged or incomplete spindle structure can signal the prevention of chromosome separation and exit from mitosis. As noted above, many chemical compounds affect microtubule stability, and thus are able to affect the cell cycle.

Recognizing the importance of research concerning cell proliferation and structure, the present invention provides compounds and methods for inhibiting the cell cycle. In general, the present invention provides compounds and pharmaceutical compositions capable of acting as inhibitors of the cell cycle. Specifically, these compounds are useful as microtubule stabilizers and/or as specific effectors of the cytoskeleton. In one aspect, the present invention provides novel compounds as shown by (10), (20), (30), (40), (50) and (6) below, and as described below. Furthermore, the present invention provides pharmaceutical compositions comprising a therapeutically effective amount of the compound having any one of the structures (10), (20), (30), (40), (50), or (60), associated with a pharmaceutically acceptable carrier.

As discussed, the present invention provides useful compositions comprising inhibitors of the cell cycle. Thus, in another aspect, the present invention provides methods of inhibiting cell cycle progression by 1) providing a system undergoing the cell cycle and 2) contacting the system with a chemical compound or composition having the general structures as disclosed herein.

In one preferred embodiment, the system undergoing the cell cycle is an in vivo system such as cells in culture or in an organism. The inventive compounds and compositions can be used to treat a subject in need of anti-proliferative agents, such as anti-cancer agents. More generally, the pharmaceutical compositions of the present invention may be administered to a subject in need of treatment with an agent that stabilizes microtubule polymerizations. Thus, the present invention also provides a method for treating a disorder comprising administering a therapeutically active composition of the present invention to a patient in need thereof.

In addition to their utility for pharmaceutical applications, the compounds of the present invention are also useful for basic scientific research purposes. For example, the compounds of the present invention that affect microtubule stability may be used to identify new cytoskeletal proteins and to unravel their regulation and function once identified. Additionally, the compounds of the present invention may be used in in vitro or in vivo mitotic assays to dissect the mitotic cycle.

Examples of inventive compounds, compositions and methods are described in more detail below. It will be appreciated that these examples are not intended to be limiting; rather all equivalents are intended to be within the scope of the present invention.

### Compounds

The present invention provides, in one aspect, several different classes of compounds capable of affecting the cell cycle.

In one preferred embodiment, the present invention provides compounds containing a trichloromethylaminal moiety functionalized with substituted or unsubstituted aryl, heteroaryl, or linear or branched alkylaryl groups having a para substituted bromine and a para-substituted sulfonamide as depicted in (10) below:

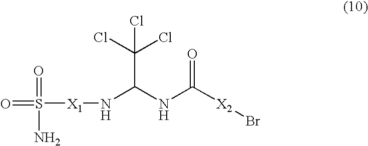

In preferred embodiments, X₁ and X₂ each independently comprise a substituted or unsubstituted aryl, substituted or unsubstituted heteroaryl, or linear or branched alkylaryl, and wherein the sulfonamide group and the bromine atom are attached to X₁ and X₂ in the para position, respectively. Novel compounds are provided by the present invention where X₁ and X₂ do not comprise unsubstituted phenyl groups. Novel pharmaceutical compositions are provided, however, for each of the abovedescribed structures, including compounds where X₁ and X₂ comprise unsubstituted phenyl groups.

In another particularly preferred embodiment of the present invention, compounds are provided having the following structure shown in (20) below:

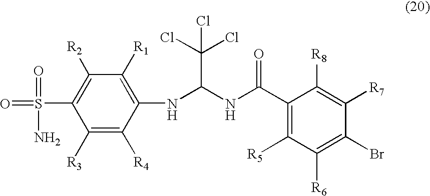

R₁-R₈ are each independently the same or different and are selected from the group consisting of H, Br, Cl, F, NH₂, CO₂H, OH, linear or branched alkyl, linear or branched acylamino, linear or branched acyloxy, linear or branched alkoxycarbonyl, linear or branched alkoxy, aryloxy, linear or branched alkylaryl, linear or branched hydroxyalkyl, and linear or branched aminoalkyl or aryl group. Each of the abovedescribed compounds represent novel compounds provided by the present invention, with the limitation that, in (20) above, R₁-R₈ cannot each simultaneously comprise hydrogen. Each of the abovedescribed compounds can be associated with a pharmaceutically acceptable carrier to provide novel pharmaceutical compositions, even when R₁-R₈ each simultaneously comprise hydrogen. In a particularly preferred embodiment, compositions are provided where R₁-R₅ are each hydrogen. Each class of compounds, as depicted by (10) and (20) above affect the cell cycle by stabilizing microtubules.

In another preferred embodiment, the present invention provides novel compounds and pharmaceutical compositions having the following general structure as shown in (30) below.

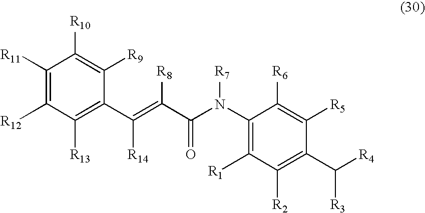

In preferred embodiments, R₁-R₁₄ are each independently the same or different and are selected from the group consisting of H, Br, Cl, F, NH₂, CO₂H, OH, linear or branched alkyl, linear or branched acylamino, linear or branched acyloxy, linear or branched alkoxycarbonyl, linear or branched alkoxy, aryloxy, linear or branched alkylaryl, linear or branched hydroxyalkyl, and linear or branched aminoalkyl or aryl group. Novel compounds are provided by the present invention where R₁, R₂ and R₅-R₁₄ are not all H when R₃ and R₄ are each methyl. Novel pharmaceutical compositions are provided however, for each of the abovedescribed structures, including compounds where R₁, R₂ and R₅-R₁₄ are all H when R₃ and R₄ are each methyl. Particularly preferred compounds and compositions include those where R₃ and R₄ each comprise methyl.

In yet another preferred embodiment, the present invention provides compounds and pharmaceutical compositions having the following structure as shown in (40):

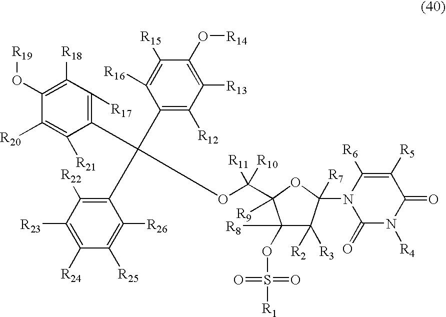

In preferred embodiments, R₁-R₂₆ are each independently the same or different and are selected from the group consisting of H, Br, Cl, F, NH₂, CO₂H, OH, linear or branched alkyl, linear or branched acylamino, linear or branched acyloxy, linear or branched alkoxycarbonyl, linear or branched alkoxy, aryloxy, linear or branched alkylaryl, linear or branched hydroxyalkyl, and linear or branched aminoalkyl or aryl group. In a particularly preferred embodiment, R₂-R₁₃, R₁₅-R₁₈, and R₂₀-R₂₆ are each hydrogen, and R₁, R₁₄ and R₁₉ are each methyl. Novel compounds are provided by the present invention where the compound does not have simultaneously R₁ as methyl, R₂-R₁₃, R₁₅-R₁₈, and R₂₀-R₂₆ as hydrogen, and R₁, R₁₄, and R₁₉ as methyl. Novel pharmaceutical compositions are provided however, for each of the abovedescribed structures, including compounds simultaneously R₁ as methyl, R₂-R₁₃, R₁₅-R₁₈, and R₂₀-R₂₆ as hydrogen, and R₁, R₁₄, and R₁₉ as methyl. Particularly preferred compounds and compositions include those where R₁ and R₁₄ each comprise methyl.

In still another preferred embodiment, the present invention provides compounds and pharmaceutical compositions having the following structure as shown in (50):

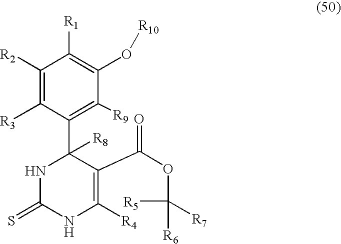

In preferred embodiments, R1-R10 are each independently the same or different and are selected from the group consisting of H, Br, Cl, F, NH₂, CO₂H, OH, linear or branched alkyl, linear or branched acylamino, linear or branched acyloxy, linear or branched alkoxycarbonyl, linear or branched alkoxy, aryloxy, linear or branched alkylaryl, linear or branched hydroxyalkyl, and linear or branched aminoalkyl or aryl group. In a particularly preferred embodiment, R₁-R₆ and R₈-R₁₀ are each hydrogen, and wherein R₇ is methyl. Novel compounds are provided by the present invention where the compound does not have simultaneously R₁-R₆ and R₈-R₁₀ each as hydrogen, and R₇ as methyl. Novel pharmaceutical compositions are provided however, for each of the abovedescribed structures, including compounds simultaneously R₁-R₆ and R₈-R₁₀ each as hydrogen, and R₇ as methyl. Particularly preferred compounds and compositions include those where R₁₀ is H.

In another preferred embodiment of the present invention provides compounds and pharmaceutical compositions having the following structure as shown in (60):

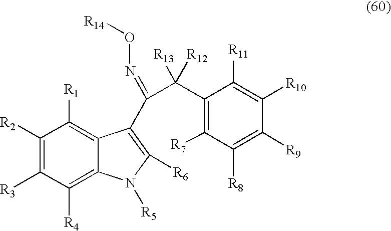

In preferred embodiments, R₁-R₁₄ are each independently the same or different and are selected from the group consisting of H, Br, Cl, F, NH₂, CO₂H, OH, linear or branched alkyl, linear or branched acylamino, linear or branched acyloxy, linear or branched alkoxycarbonyl, linear or branched alkoxy, aryloxy, linear or branched alkylaryl, linear or branched hydroxyalkyl, and linear or branched aminoalkyl or aryl group. In a particularly preferred embodiment, R₁-R₄ and R₆-R₁₄ are each hydrogen, and R₅ is methyl. Novel compounds are provided by the present invention where the compound does not have simultaneously R₁-R₄ and R₆-R₁₄ each as hydrogen, and R₅ as methyl. Novel pharmaceutical compositions are provided however, for each of the abovedescribed structures, including compounds simultaneously R₁-R₄ and R₆-R₁₄ each as hydrogen, and R₅ as methyl. Particularly preferred compounds and compositions include those where R₅ is methyl.

Each of the compounds (30)-(60) shown above are capable of interfering with the cytoskeletal structure of cells undergoing mitosis.

Furthermore, as will be appreciated by one of ordinary skill in the art, the present invention is intended to include all enantiomers and diastereomers of the inventive compounds utilized in the compositions and methods.

### Pharmaceutical Compositions

The compounds disclosed herein inhibit cell cycle progression by either 1) acting on microtubules or 2) effecting the mitotic cytoskeleton, and thus may be used to treat a variety of human conditions including a broad range of cancers and pathogenic infections. As noted above microtubule stabilizing agents may be used to prevent or reduce atherosclerosis or restenosis. Furthermore, compounds of the present invention may be used as immunosurpressants or as morning-after pills. Thus, the present invention provides pharmaceutical compositions comprising any one of the abovedescribed compounds (10), (20), (30), (40), (50), or (60) and a pharmaceutically acceptable carrier. Methods for treating disorders are also provided comprising administering a therapeutically effective amount of an inventive composition to a patient in need.

As will be appreciated by one of ordinary skill in the art, pharmaceutical compositions may be constituted into any form suitable for the mode of administration selected. For in vivo delivery (i.e., into a cancer patient), it is preferred that the delivery agent be biocompatible and preferably biodegradable and non-immunogenic. In addition, it is desirable to deliver a therapeutically effective amount of a compound in a pharmaceutically acceptable carrier. For example, it is known that one may inject a compound into a patient in a buffered saline solution. Injection into an individual may occur intravenously, intramuscularly, or for example, directly into a tumor. Alternatively, in vivo delivery may be accomplished by use of a syrup, an elixir, a liquid, a tablet, a time-release capsule, an aerosol or a transdermal patch.

It follows that it is desirable to deliver the compound into a cell or population of cells (i.e. after in vivo into individual). Any delivery agent that is biocompatible and preferably biodegradable and non-immunogenic, as mentioned above, that interacts with the compound to be delivered in such a way as to mediate its introduction into the cell for use in the present invention. Of course one of ordinary skill in the art will recognize that delivery of the compositions of the present invention in any manner that maintains their biological activity in vivo is acceptable. For example, it may be desirable to select a delivery agent with a high charge density so that it is able to interact with a particular compound. Furthermore, the inventive compounds and pharmaceutical compositions may be functionalized with targeting agents well known in the art and thus selectively deliver the pharmaceutical compositions of interest to desired cellular targets.

As will be appreciated by one of ordinary skill in the art, pharmaceutical compositions may be constituted into any form suitable for the mode of administration selected. Suitable for oral administration include solid forms, such as pills, capsules, granules, tablets, and powders, and liquid forms, such as solutions, syrups, elixirs and suspensions. Forms useful for parenteral administration include sterile solutions, emulsions, and suspensions.

The drug may otherwise be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings.

Optimal dosages to be administered may be determined by those skilled in the art, and will vary with the particular compound in use, the strength of the preparation, the mode of administration, and the advancement of the disease condition. Additional factors depending on the particular patient being treated will result in a need to adjust dosages, including patient age, weight, gender, diet, and time of administration.

### Applications

In addition to the therapeutic applications described above, the compounds of the present invention are also useful for research concerning the cell cycle. Thus, the present invention provides methods for elucidating cellular mechanisms comprising 1) providing a system undergoing the cell cycle, and 2) contacting the system with a chemical compound with the structures (10), (20), (30), (40), (50), and (60) depicted herein. The system to be studied may be any in vitro or in vivo system available in the art.

Clearly, compounds that act as cell cycle inhibitors are invaluable to the study of the cell cycle pathway. In general, inhibitors of cell cycle progression are essential as tools that can be used to achieve arrest at specific points in the cell cycle. This allows one to administer the reagent to a population of cells to achieve synchronization of the mitutic cell cycle. In addition, specific proteins or activities may be identified as being essential to cell-cycle-related functions by their interaction with small molecule inhibitors of the cell cycle. Proteins that play an important role downstream of the direct target may be confirmed by indirect inhibition by the same agent. In essence, exposure of cells to such reagents causes a conditional loss of function in the target protein in a similar manner to that achieved by the use of temperature-sensitive mutations in a gene. Similarly, such inhibitors of microtubule polymerization and depolymerization may be used to identify new cytoskeletal proteins and unravel the function and regulation of cytoskeletal proteins.

Additionally, as will be appreciated by one of ordinary skill in the art, any in vitro assay may be used to monitor inhibition at different mitotic transition points, when the cell cycle progresses from one phase to the next. This may be accomplished by altering the timing of addition of the chemical compound in question to the mitotic extract. Alternatively it may be desirable to test whether certain compounds inhibit mitosis at early transition stages (e.g., prophase or anaphase). According to one preferred embodiment of the present invention, the test compound is added to the interphase mitotic extract simultaneously with the Delta90 cyclin protein (and thus at the onset of mitosis) to test for successful of inhibition of early transition stages. Another aspect of the invention tests whether certain compounds inhibit mitosis at late transition stages (e.g., microtubule assembly and disassembly and chromosome segregation). Thus according to other preferred embodiments of the present invention, the test compound is added after Delta90 cyclin so that mitosis has progressed past the early transition stages and inhibition of ubiquitin degradation can be assessed. Effectors of microtubule stability are particularly desirable compounds according to the present invention. Identification of such compounds are likely to allow further dissection of key regulatory steps of the mitotic pathway, cytoskeletal organization and serve as important tool in various other research and therapeutic purposes as mentioned above.

Indeed, compound (50) has already been shown to interact with the mitotic protein ET5 and thus acts as a mitotic motor.

### Materials and Methods/Results

Library Composition

A total of 16,320 compounds obtained from Chembridge Corporation, San Diego Calif. All compounds present as 5 mg/ml stock concentrations. General features of the library: 1691 hydrazones, 2500 nitro-containing compounds, 1075 sulfonamides, 77 nitrile compounds 681 thioureas, 240 triazine-containing compounds, 2500 imines, 703 ureas, 502 trichloroaminals (partial list). The average molecular weight was around 300.

### Detection

A detection assay providing an indirect measure of the mitotic index of a population of cells was used to identify compounds that act as cell cycle inhibitors. By measuring the level of phosphorylation of a chromatin-associated protein using the anti-phosphonucleolin (TG-3) cytoblot (Stockwell et al. 1999), nucleolin the mitotic index (i.e., the number of cell in mitosis) was assessed. In brief, the library compounds were dissolved in DMSO (5 mg/ml) and pin-transferred from 384-well plates (Library plates #1-51) for a final concentration 5-10 ug/ml using a 384-polypropylene pin array (Genetix) into 384-well screening plates (Nunc) seeded with 4,000 A549 human lung carcinoma cells (ATCC) in DMEM+ and incubated for 22 hours at 37° C. with 5% CO₂. Compounds from library plates #1-24 were pooled at two compounds per well, while compounds from library plates #25-51 were tested singly. After 22 hours screening plates were processed as described in Stockwell et al. (1999) except a 1:500 dilution of the anti-phosphonucleolin (TG-3) monoclonal antibody (gift of Dr. Peter Davies) was used. The reactivity of the anti-phosphonucleolin antibody was measured using enhanced chemiluminescent detection (Amersham) after incubation of each well with a horseradish peroxidase-coupled anti-mouse IgM secondary antibody (Sigma) and addition of luminol. The relative luminescence produced from each well was measured on an automatic plate reader (LJL Analyst). Compounds corresponding to wells which showed greater than 3-fold the luminescent signal as compared to DMSO treated (control) wells were re-arrayed and re-tested using pin-transfer.

### Initial Screen

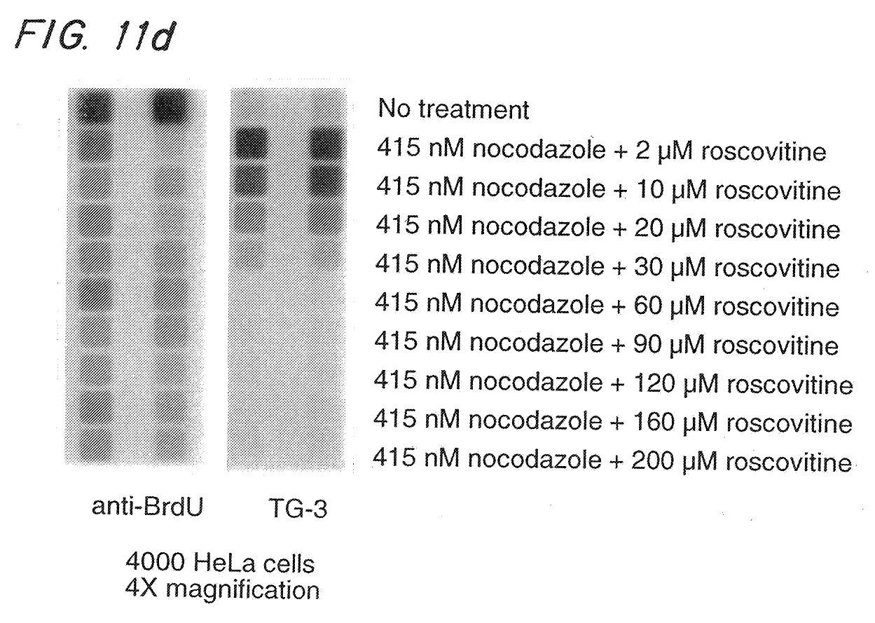

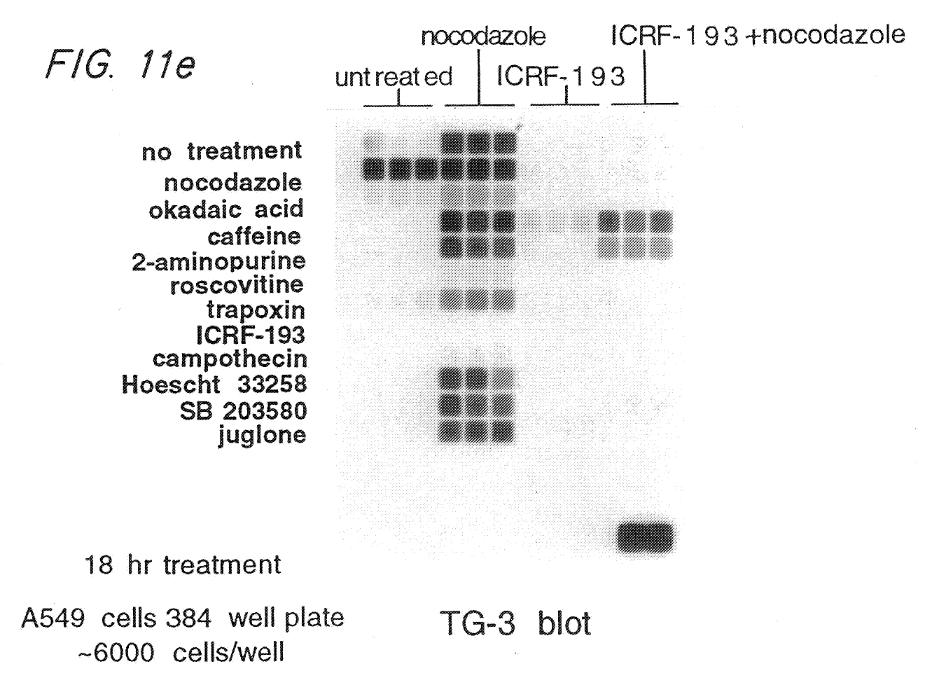

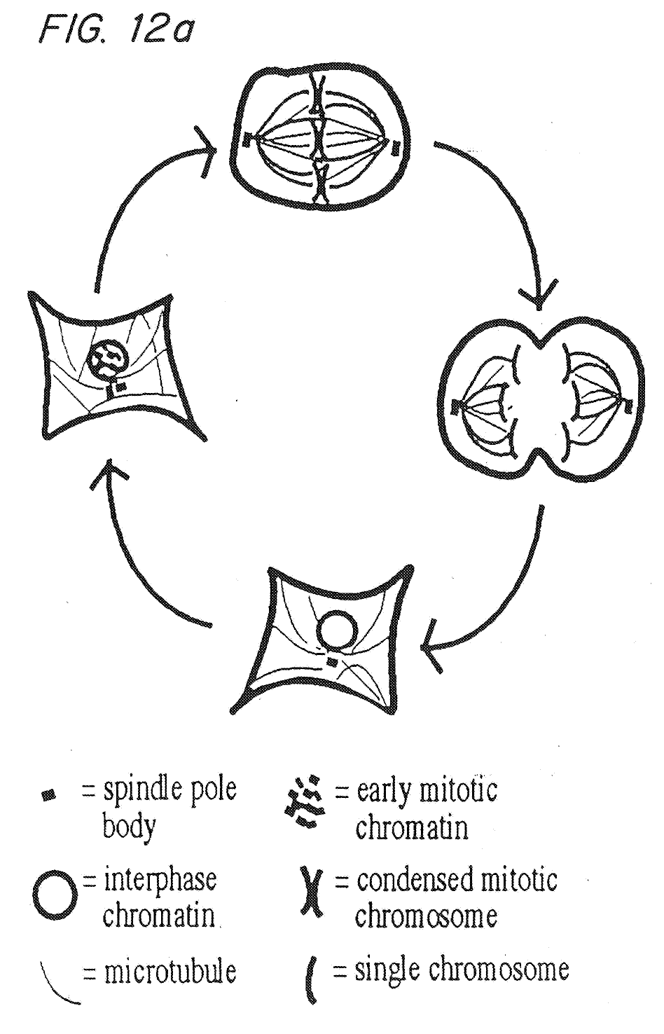

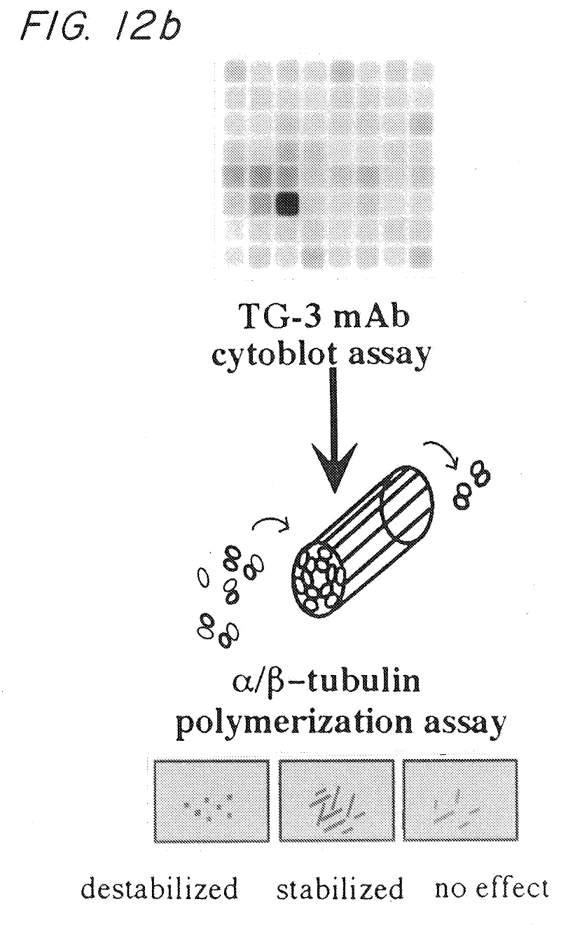

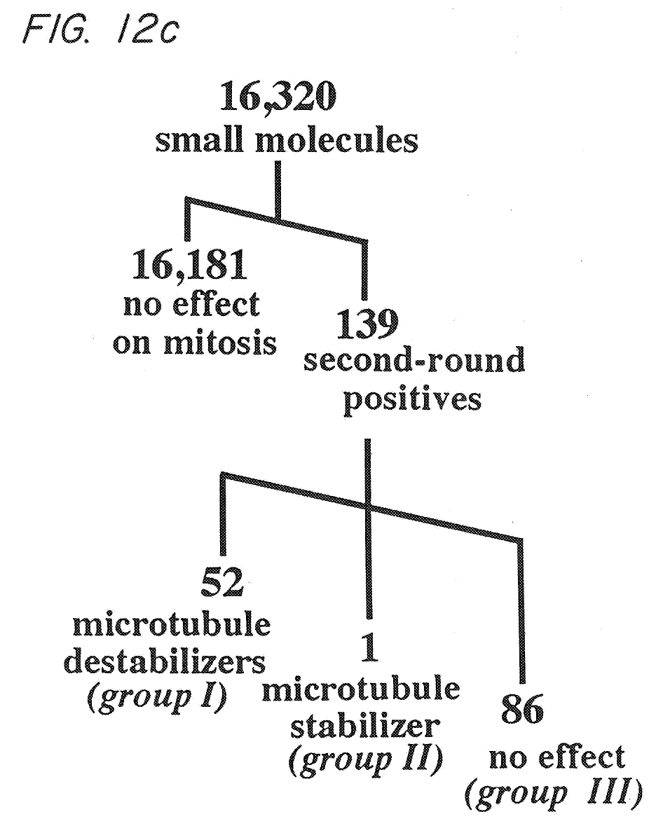

In order to identify small molecules that affect progression of mammalian cells through mitosis, the 16,420 compounds composing the library were screened using an anti-phosphonucleolin (TG-3) cytoblot. The first round positives were selected and tested in duplicate at four different concentrations (25-24 μM) on two separate plates as described above. Compounds testing positive a second time were re-tested, and if positive a second time, chosen for subsequent analysis. In these two rounds of screening, 139 compounds were identified that increased the amount of phosphorylated nucleolin in asynchronous A549 lung epitheliel cells at least 2.5 fold (FIG. 12). Additional experiments revealed that these compounds had no effect on the in vitro polymerization of actin, the in vitro degradation of a cyclin B-luciferase fusion protein in Xenopus extracts, or the activation of a growth-factor-dependent reporter gene, indicating a level of specificity in their target interactions.

### Secondary Screen

To determine whether any of the compounds identified in the initial screen directly acted upon microtubules each were tested in vitro for effects on the polymerization dynamics of pure tubulin. In this assay a mixture containing pure tubulin (purified from calf brain), tetramethylrhodamine-labeled tubulin and GTP was incubated for 15 min at 37° C. in the presence of DMSO (control) or the compounds at a final concentration of 20 μg/ml. As a control for a destabilizing and stabilizing compound we used 20 μM nocodazole and 20 μM taxol (Paclitaxel™) respectively. The samples were fixed with glutaraldehyde and the abundance and length of fluorescence-labeled microtubules were observed using fluorescent microscopy.

52 compounds (group I) were identified that destabilized microtubules, and one (group II; compound 9a in FIG. 24) was identified that stabilized microtubules when assayed in this format. The remaining 86 compounds (group III) had no discernible effect.

### Characterization of Group I

The most potent compounds in group I, based on the cytoblot assay, are members of the structural types 1-8 (FIG. 24a). Compound 1a is the well-known microtubule-destabilizer nocodazole (Hamel, Med. Res. Rev. 2:207, 1996). This compound was present twice within the library, along with an analog 1b, each of which had been included in group I. Six out of the 52 group I compounds (2a-f) share the same three-ring skeleton, although their dose-response in the cytoblot assay varied. This allowed us to compare the phenotypic effects of weak vs. strong destabilizers using fluorescence microscopy and cells stained for microtubules and chromatin (Hyman et al., Met. Enzymol. 196:478, 1991). Compounds having EC₅₀'s in the range of 0.5-1 μM in the cytoblot assay (e.g., 2b, 3b) completely destabilized microtubules, in both interphase (non-dividing) and mitotic cells, resulting in randomly arrayed mitotic chromosomes. Compounds having EC₅₀'s in the range of 5-10 μM (e.g., 2e) either partially destabilized interphase microtubules or had no visible effect on the microtubule cytoskeleton. Regardless of their effects on interphase cells, these less potent compounds still caused abnormal mitotic spindle structures and altered chromosome distribution.

Fluorescence microscopy of cells treated with high concentrations (about 50 μM) of 23 analogs related to 2, which either scored negatively in the cytoblot assay or were not within group I, expanded the number of small molecules that destabilize microtubules in the cells to include another 11 compounds (see FIG. 25). As compounds 2g, 2i, 2o, and 2q were among those considered to be in group III, this indicates that a subset of group III compounds may also target tubulin directly, but may act weakly and thus were ineffective at targeting purified tubulin. For example, while compound 2g had no effect on the stability of purified microtubules or on the microtubule cytoskeleton of interphase cells, mitotic cells show a shorter, disarrayed spindle and misarranged chromosomes compared to the bormal bipolar spindle and alignment of chromosomes.

Although the discovery of small molecule inhibitors of protein-protein interactions is in general demanding, we note the significant occurrence (approximately 0.3% of compounds screened) of inhibitors of α-tubulin-β-tubulin interactions in this study. This illustrates the use of a phenotype-based screen to identify components in a pathway that are most easily targeted by small molecules.

### Characterization of Group II

Like taxol (Hamel, Med. Res. Rev. 2:207, 1996; Schiff et al., Proc. Natl. Acad. Sci. USA 77:1561, 1980; Amos et al., Chem. Biol. 6:R65, 1999) and other natural product stabilizers (e.g., discodermolide; Hamel, Med. Res. Rev. 2:207, 1996; ter Haar et al., Boichem. 35:243, 1996; Hung et al., Chem. Biol. 3:287, 1996), compound 9a (group II) (FIG. 24b), here named synstab A (for synthetic stabilizer), stabilized microtubules formed from the polymerization of purified α- and β-tubulin in vitro. In the cytoblot assay, synstab A has an EC₅₀ of 10-15 μM, whereas a structurally related compound lacking the sulphonamide has no effect. This cytoblot EC₅₀ is approximately 500-fold greater than taxol. Consistent with the stabilizing effects of synstab A on purified microtubules, staining of kidney epithelial cells (BS-C-1) showed that synstab A-treatment leads to microtubule bundles in interphase cells and to disrupted spindles and abnormal chromosome distribution in mitotic cells. The analog of synstab A lacking the terminal sulphonamide has neither of these effects. Since removing synstab A after a 2 h treatment by washing restored the normal microtubule staining pattern in both interphase and mitotic cells, the observed effects of synstab A are reversible and are not due to covalent modification of tubulin. The reversible bundling of interphase microtubules and the reversible effects on mitotic cells are reminiscent of those resulting from both discodermolide- and taxol-treatment of cells.^{10,11} We note, however, that the bundling of microtubules induced by taxol is less pronounced than that induced by synstab A in BS-C-1 cells or than that induced by taxol in other cell lines (ter Haar et al., Boichem. 35:243, 1996; Hung et al., Chem. Biol. 3:287, 1996).

Since taxol and discodermolide prevent cold depolymerization of microtubules (Hung et al., Chem. Biol. 3:287, 1996), we tested whether synstab A-treatment of cells would cause a similar effect. While taxol-treatment (10 μM, 4 h) noticeably stabilized microtubules, synstab A-treatment (25 μM, 4 h) only partially protected cells from cold depolymerization (4° C., 0.5 h), resulting in a slight increase in the number of remaining microtubules as compared to untreated cells. We next determined that the binding of a fluorescently labeled version of taxol to microtubules could be competed for by the addition of synstab A. This suggests that synstab A either binds to the same or overlapping binding sites on microtubules, or induces a conformational change that prevents taxol-binding. This mutually exclusive binding is consistent with the observed taxol-like effects of synstab A on cells and on purified microtubules.

In agreement with the phenotypic effects of synstab A observed through fluorescence microscopy, fluorescence-activated cell sorting confirmed that, similar to cells treated with nicodazole or taxol, cells treated with synstab A had fully replicated chromosomes (4N DNA content) and increased TG-3 staining. In addition, immunoblotting of total cell extracts derived from cells treated with taxol or with systab A at concentrations that do not affect viability show increased TG-3 reactivity.

Although synstab A shares many of the functional properties of taxol, it does not share structural features of known, natural product stabilizers of microtubules (taxol, discodermolide, epothilone, and eleutherobin). The ease with which synstab A was identified and its simple structure suggest to us that screening-based approaches to taxol-like compounds may prove more effective than design-based approaches using “pharmacophore” models (Ojima et al., Proc. Natl. Acad. Sci. USA 96:4256, 1999; Wang et al., Org. Lett. 1:43, 1999).

### Characterization of Group III

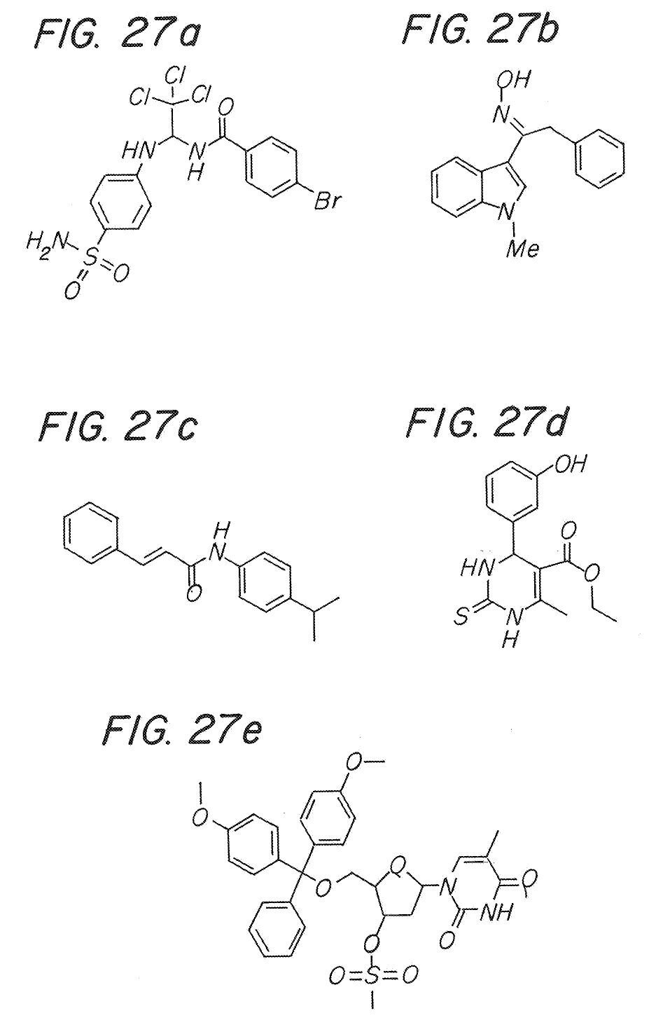

Group III compounds of particular interest are shown in FIG. 27, Panels C-E.

We investigated the phenotype of mammalian cells (BS-C-I) treated with the compounds of group III. Live images were taken as described previously (Cramer et al., Curr. Op. Cell Biol. 6:82, 1994); for immunofluoresence, the cells were stained with a Golgi-specific antibody (anti-Golgi 58K protein antibodies [Sigma]) or with anti-α-tubulin antibodies (DM1 A [Sigma]); actin was visualized using TRIC-conjugated phalliodin (Sigma); lysosomes were stained with LysoTracker (Molecular Probes; Palmiter et al., EMBO J. 15:1784, 1996). Our examination of the distribution of microtubules, actin, and chromatin in fixed cells by fluorescence microscopy allowed us to divide the small molecules into three classes. Twenty-seven had no observable effect on the microtubule and actin cytoskeleton or on cheomosome distribution. Consistent with the data from the cytoblot assay we observed an increase in the number of normal appearing mitotic cells. These compounds may increase the mitotic index by perturbing the function of proteins that regulate progression through the cell cycle, e.g., anaphase regulators, rather than structural or mechanochemical components of the mitotic spindle. It is also possible that these compounds have a subtle effect on cytoskeletal dynamics or chromosome organization that may not be observable in fixed cells.

Forty-two compounds affected cells in interphase as well as mitosis. Cells treated with these small moleules had disorganized or partially depolymerized interphase microtubules, in addition to abnormal spindle structures and misaligned chromosomes, although the actin cytoskeleton was not affected. Five small molecules altered the mitotic spindle, but not microtubules, actin filaments, or chromatin in interphase cells even at high concentrations. The mitotic phenotypes caused by these small molecules included chromosome misalignment, loss of spindle pole organization, changes in spindle shape, and combinations of these.

The phenotype resulting from the treatment of cells with a 1,4 dihydropyrimidine-based compound (DHP) was especially interesting. In treated cells, the bipolar spindle was replaced with a monoastral microtubule array surrounded by a ring of chromosomes. Interphase cells were not affected. Over 90% of the mitotic cells displayed the monoastral phenotype after treatment with this small molecule. We refer to this compound as monastrol (FIG. 26D). Monastrol used in all experiments subsequent to the screen was synthesized using published protocols (Lewandowski et al., J. Comb. Chem. 1:105, 1999).

Normal bipolar spindles are thought to assemble in part through interaction between anti-parallel microtubules from the two half-spindles (FIG. 26A; Rieder et al., Trends Cell Biol. 8:310, 1998).

### Tertiary Screen

Three different assays were used to confirm microtubule-stabilizing effects in the in vitro tubulin polymerization assay by repeating the above experiment. In addition, three independent experiment assessments were carried out. 1) In vitro competition experiments with fluorescence-labeled taxol. In brief, using an oregon green labeled version of taxol and a fluorescence polarization assay, we were able to compete off bound labeled taxol with compound 1A. 2) In vivo incubation of BSCI-cells (a monkey epithelial cell line) with 40 μM 1A for four hours. In brief, BSCI cells were seeded on glass coverslips and cultured overnight. Compound 1A (40 uM) was added for 4 hours and subsequently the cells were stained. Unlike control treated cells which showed no change, cells exposed to 1A showed bundling of interphasic microtubules and aberrant mitotic spindles with misaligned chromosomes. 3) treatment of cells with 1A resulted index by the phosphonucleolin (TG-3) cytoblot as described above as measured visually.

Without these interactions the spindle remains monoastral, and the cell is arrested in mitosis, presumably because the unattached kinetochores activate the mitotic checkpoint. Mitotic kinesins have been implicated in anti-parallel overlap interactions (Walczak et al., Curr. Biol. 8:903, 1998). Inhibition of the tetrameric mitotic kinesin Eg5 with antibodies induced monoasters (Sawin et al., Nature 359:540, 1992; Blangy et al., Cell 83:125, 1995; Sharp et al., J. Cell Biol. 144:125, 1999). We therefore hypothesized that Eg5 might be a target of monastrol. Like other motile kinesins, Eg5 can drive in vitro microtubule gliding (Sawin et al., Nature 359:540, 1992; Kapoor et al., Proc. Natl. Acad. Sci. USA, 1999). We tested whether monastrol inhibits Eg5 motility in vitro. Intriguingly, monastrol inhibited the Eg5 driven microtubule motility with an IC₅₀ (inhibitory concentration) of 14 PM, which is comparable to its EC₅₀ (effective concentration) of 22 PM in the cytoblot assay. Microtubule attachment to the Eg5 coated coverslip was maintained in the presence of monastrol (FIG. 26C). Washout experiments demonstrated that the effect of monastrol is reversible in vitro and in vivo. To test whether inhibition of the Eg5 driven microtubule gliding is specific to monastrol, we tested the closely related compound DHP2 (FIG. 26D). DHP did not arrest cells in mitosis or generate monoastral spindles. Identical concentrations of this compound had no significant effect on the Eg5-dependent microtubule velocity in vitro (FIG. 26C).

Eg5 is a member of the kinesin superfamily, which includes over 100 homologous proteins involved in organelle transport, membrane organization, and assembly and maintenance of mitotic spindles (Vale et al., Annu. Rev. Cell. Dev. Biol. 13:745, 1997). To test whether monastrol affects other motor proteins, we first assayed its ability to inhibit microtubule gliding driven by conventional kinesin in vitro. The N-terminal motor domain of conventional kinesin shares 33% sequence identity with the Eg5 motor domain (Kapoor et al., Proc. Natl. Acad. Sci. USA, 1999). We detected no inhibition of kinesin-driven microtubule gliding by monastrol at 200 μM (FIG. 26F). We next investigated whether monastrol disturbs the distribution of lysosomes or the Golgi apparatus in vivo. The intracellular localization of these organelles is thought to depend on the activity of multiple motor proteins; perturbation of these motor proteins is expected to result in organelle mislocation (Hirokawa, Science 279:519, 1998). Monastrol concentrations that caused strong spindle defects did not affect localization and organization of the Golgi apparatus or lysosomes in interphase cells. Taken together, these results demonstrate that monastrol is not a general inhibitor of motor proteins or their regulators.

We then asked whether the chromosomes surrounding the monoastral microtubule array maintain microtubule attachment. Chromosomes attached to spindle microtubules of spontaneous monoastral spindles display a stereotyped movement away from and towards the pole, reflecting the activity of kinetochore proteins, kinesins and microtubule polymerization dynamics (Rieder et al., J. Cell Biol. 103:581, 1986). Live images of monastrol-treated BS-C-1 cells revealed that the chromosomes show typical oscillatory behavior, indicating that monastrol does not inhibit chromosome attachment to microtubules.

We refer to this approach of phenotype-based small molecule screening as chemical genetics, because of its conceptual similarity to classic forward genetic screens. The cytoblot assay will be a key tool for chemical genetics. Using appropriate antibodies, it can provide a quantitative readout of essentially any post-transnational modification of a specific protein in the cell. In this Example, a cytoblot assay for phosphorylation of nucleolin was used as a readout of mitosis, and our screen detected compounds that arrest cells in mitosis. After eliminating compounds that targeted pure tubulin, a sufficiently small number of the original 16,320 compounds remained for us to use a systematic visual analysis. For monastrol, the information from such analyses facilitated the identification of the kinesin Eg5 as a cellular target. Previously the only known small molecule kinesin inhibitors were 5′-adenylylimido-diphosphate (AMP-PNP) (Saxon, Met. Cell Biol. 44:279, 1994) and a marine natural product (Sakowicz et al., Science 280:292, 1998), both of which are not cell-permeable and affect multiple kinesin family members. Monastrol, in contrast, is the first example of a cell-permeable compound that selectively perturbs the function of a motor protein essential for mitosis. Other motor proteins involved in lysosome and Golgi distribution seem not to be affected by other mechanisms have shown anti-tumor activity in humans (Jordan et al., Met. Enzymol. 298:252, 1998), monastrol may serve as a lead for anti-cancer drugs. Monastrol will, however, be a valuable tool for dissecting the function of Eg5 in the establishment of spindle bipolarity and other cellular processes.

### Example 12

### Functional Fingerprinting of Small Molecules

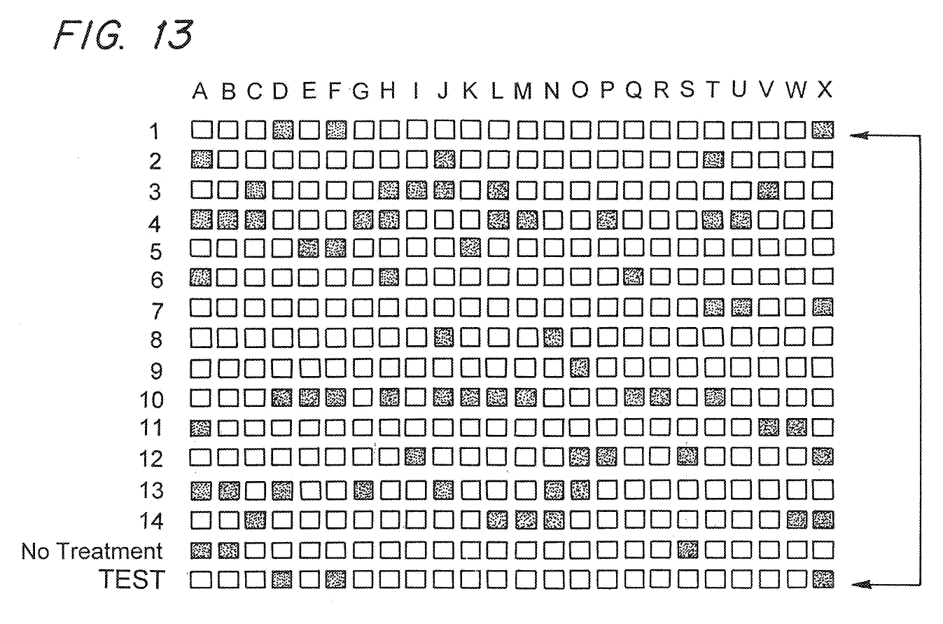

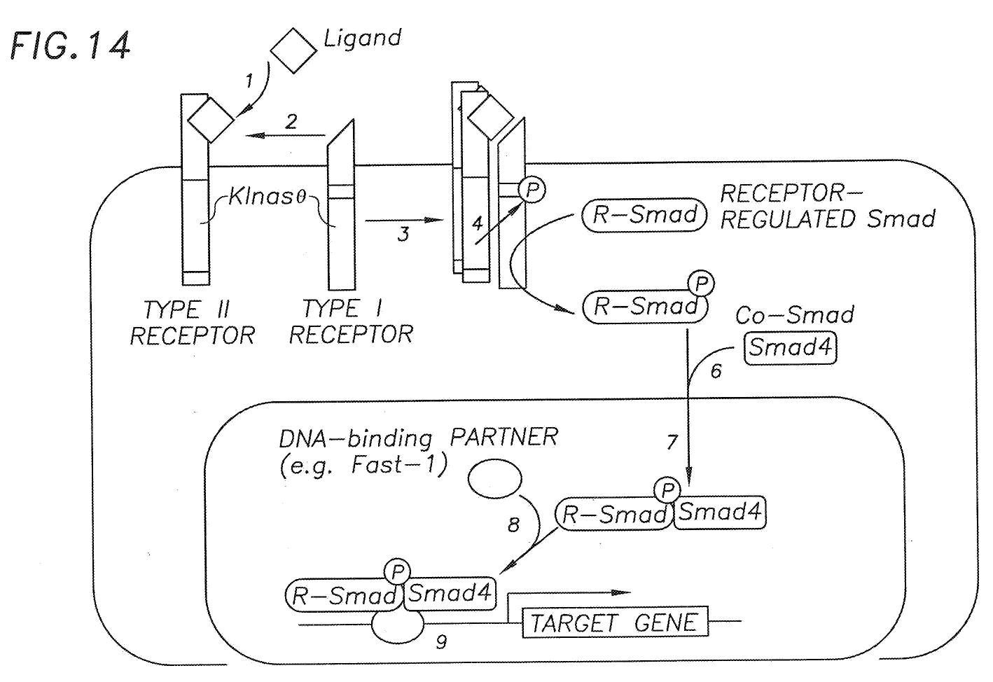

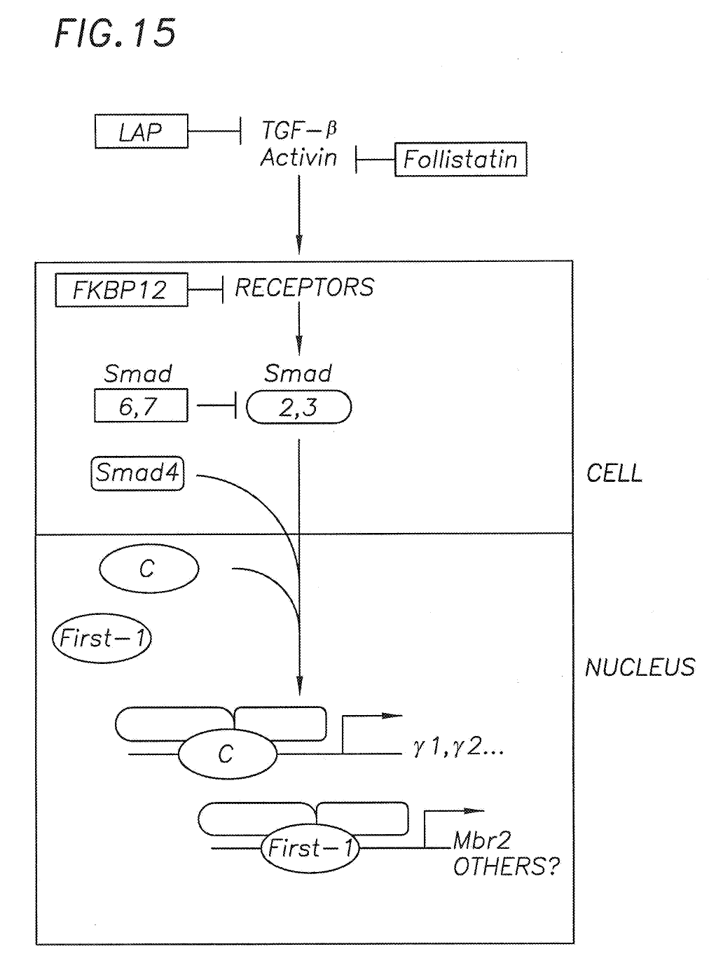

A single cytoblot array can be used to detect many different alterations in cellular activity induced by a particular biological agent or small molecule (see FIG. 13). A master 6144, 1536 or 384 well plate was created with 6144, 1536 or 384 different antibodies, one antibody per well. A second 6144, 1536 or 384 well plate (the test plate) was seeded with 100, 500 or 2000 cells, respectively. Each well was treated with the same bioactive agent (the test compound). Alternatively, a set of 24, 48 or 96 master antibodies was prepared for use in a single row of 384, 1536 or 6144 plates, respectively. In this case, each row was used to test a different known or unknown small molecule or biologically active agent and one row was left untreated (see FIG. 13). The cells were fixed and aliquots of the master antibody stocks were transferred to each well of the test plate during the cytoblot procedure. The antibodies were detected with a secondary antibody coupled to HRP and HRP retention on the cells is detected with luminol, hydrogen peroxide and the enhancer p-iodophenol. Thus, in one plate, up to 6144 different cellular components can be detected, thereby giving a large amount of information about the test compound and its possible mechanism of action. The total profile of these alterations in cellular components is characteristic of each known bioactive small molecule and therefore provides and effective “fingerprint” of a given small molecule. By comparing the fingerprints of bioactive agents with known and unknown mechanisms, one can potentially learn about the mechanism of action of new compounds. This information can be used to categorize functionally new biologically active agents.

### Example 13

### Chemical Genetics

The immunosuppressive natural product FK506 was assayed for its ability to act as a suppressor of rapamycin. This particular experiment relies on the fact that FK506 and rapamycin share a common binding protein, FKBP12 (FK506 and rapamycin binding protein, 12 kilodaltons). As a heterodimeric complex, rapamycin/FKBP12 binds to the protein, FRAP (FKBP12-rapamycin associated protein). Alternatively when FKBP12 is complexed with FK506, this heterodimer binds to the phosphatase calcineurin. Since the antiproliferative effect of rapamycin is dependent on the formation of the rapamycin/FKBP12 complex, excess FK506 could potentially prevent rapamycin-induced growth arrest by titrating away FKBP12, thus preventing the formation of the rapamycin/FKBP12 complex.

This suppression of the ability of rapamycin to inhibit growth is demonstrated in the cytoblot assay by the simultaneous treatment of mink lung cells with rapamycin and excess FK506. The excess FK506 should result in the ability of cells to incorporate BrdU in the presence of rapamycin. Mink lung cells were seeded into 384-well plates and treated with varying concentrations of both rapamycin and FK506 together for 16 hours and then treated with rapamycin, FK506 and BrdU for 16 hours. FIG. 7 shows that a 30-100 fold excess of FK506 suppresses the antiproliferative effect of rapamycin. Thus, the cytoblot assay is capable of detecting small molecule suppressors of antiproliferative agents. This suppressor screening strategy can be applied to other antiproliferative agents, including but not limited to ones such as TGF-β, hydroxyurea, nocodazole, mimosine, benomyl, trapoxin, trichostatin and depudicin.

### Example 14

### Identification of Metal Binding Compounds that Activate TGFβ-Responsive Genes

### Background

Transforming growth factor β (TGFβ) is a multifunctional polypeptide signaling factor that regulates cell differentiation, proliferation, and apoptosis (see, for example, Roberts et al., eds, Peptide Growth Factors and Their Receptors, Springer-Verlag, Heidelberg, 1990). Multiple genes in the TGFβ pathway are mutated in human cancers, implicating dysregulation of this signaling pathway in the genesis of tumors (see, for example, Reiss, Oncol. Res. 9:447, 1997). The TGFβ pathway may also be involved in blood clotting, immunosuppression, and the prevention of inflammation (see, for example, Hardman et al., eds., Goodman and Gillman's The Pharmacological Basis of Therapeutics, 9th Ed., McGraw Hill, New York, N.Y. 1996).

Given its diverse and medically significant biological roles, TGFβ is an attractive target for pharmaceutical research. There is a need for the identification of agents that mimic one or more of the activities of TGFβ.

### Materials and Methods

REAGENTS: 16,320 structurally diverse compounds were obtained from Chemridge Corporation (San Diego, Calif.) as 5 mg/mL dimethylsulfoxide (DMSO0 stock solutions. 2,2′-(methylimino)bis-8(quinolinol) (1a) (Fluka; Milwaukee, Wis.; cat #67585) was stored at −20° C. as a 7.5 mM DMSO stock solution (3000×). The related dimeric 8-quinolinols 1b-c (Chembridge Corporation; cat#175091, 175093) were stored at −20° C. as 7.5 mM DMSO stock solutions. Benzoic acid, 4-hydroxy, [(2-hydroxyphenyl)methylene]hydrazide (2) (Chembridge Corporation; cat #112930) was stored at −20° C. as a 50 mM DMSO stock solution (500×). Transforming growth factor beta (TGF-β) (Sigma Corporation; St. Louis, Mo.; cat#T-1654) was stored in 20 μL aliquots at −80° C. as 40 nM stock solutions (100-1000×) in 0.2 μm-filtered 4 mM HCl with 1 mg/mL bovine serum albumin (Sigma; cat#A-2153). 5-Bromodeoxyuridine (Sigma; cat#B-9285) was stored at 4° C. as a 10 mM stock solution (1000×) in phosphate buffered saline, pH 7.4 (PBS). Diethyldithiocarbamate (DDC) (Sigma; cat#D-9428) was stored at −20° C. as a 1M stock solution in DMSO. 8-quinolinol (8-hydroxyquinoline; Lancaster; Windham, N.H.; cat#2529) was stored at −20° C. as a 50 mM stock solution in ethanol. 2,2′-dipyridylamine (Aldrich Chemical Co.; Milwaukee, Wis.; cat#D21, 640-2) was stored at −20° C. as a 1M stock solution in DMSO. H₂O₂ (Mallinckrodt Baker, Inc.; Phillipsburg, N.J.; cat#5240) was stored at 4° C. as a 30% aqueous solution. 2,2′-azobis (2-methylpropinonitrile) (AIBN; Morton Thiokol, Inc.; Danvers, Mass.; cat#13290) was stored at 4° C. as a 50 mM stock solution in DMSO. Anti-pan TGF-β neutralizing antibody (Sigma; cat# T-9429) was stored in 0.2 μm-filtered PBS as a 1 mg/mL stock solution (100×). The following metal salts were used: ZnCl₂ (Mallinckrodt Baker, Inc.; cat#8780-03), FeCl₃ (Aldrich; cat# 15,774-0), CuC1 (Aldrich; cat# 22, 962-8), CuC1 (Mallinckrodt Baker, Inc.; cat# 1862-1), A1C1₃ (Aldrich; cat# 29, 471-3), MnC1₂.4H₂O (Mallinckrodt Baker, Inc.; cat# 2540-01), CoCl₂.6H₂O (Mallinckrodt Baker, Inc.; cat# 4532), NiSO₄.6H₂O (Aldrich; cat# 22, 767-6), MgCl₂ (Aldrich; cat#24, 413-9)KCl (Mallinckrodt Baker, Inc.; cat#6858), CaCl₂.2H₂O (Mallinckrodt Baker, Inc.; cat#4160), NaCl (Fisher Scientific; Fair Lawn, N.J.; cat#S671-3), Ba(OAc)₂ (Aldrich; cat#24, 367-1).

PLASMIDS: The plasmid p3TPLux, which contains three copies of the phorbol myristate acetate TGFβ response element from the collagenase gene as well as a TGFβ-responsive fragment of the plasminogen activator inhibitor type 1 promoter, was obtained from Joan Massague (Corcanno, et al., Mol Cell Biol 15:1573, 1995). The plasmid pNFk-B-Lux was purchased from Stratagene (LaJolla, Calif.; cat #219077).

CELL LINES: Mv1Lu mink lung epithelial cells were obtained from the American Type Culture Collection (Manassas, Va.; cat#CCL64). 6F mink lung cells, a stably-transfected clone containing p3TPLux as well as another plasmid, are derived from Mv1Lu cells. The generation of this clone was described previously by us (Stockwell, et al., Cell Biol. 8:76; 1998). Both Mv1Lu and 6F cells were cultured in 10% mink medium, which consists of Dulbecco's Modified Eagle Medium (DMEM; GibcoBRL, Gaithersburg, Md.; cat#1 1995-040) with 10% fetal bovine serum (FBS; GibcoBRL; cat#10438-026), 100 units/ML penicillin G sodium (GibcoBRL; cat#15140-122), 100μ/mL each of the amino acids L-alanine (Sigma; cat#A-3534), L-aspartate (Sigma; cat# A-4534), L-glutamine (Sigma; cat#G-7029), glycine (ICN Biomedicals, Inc.; Aurora, Ohio; cat#100570), L-asparagine (Sigma; cat# A-4284), and L-proline (Sigma; cat#P-4655). 700 μg/ml G418 sulfate (GibcoBRL; cat#11811-031) was added to cultures of 6Fcells. FATZ Jurkat T-cells, which contain a stably-integrated NFAT-lacZ reporter gene and were described previously by Fiering et al. (Genes Dev. 4:1823, 1990) were obtained from Gerald Crabtree, and cultured in RPM1 medium 1640 (1×) (GibcoBRL; cat# 11875-085) with 10% FBS, 100 units/mL penicillin G sodium, 100 μg/ML streptomycin sulfate, and 2 mM L-glutamine (GibcoBRL; cat#25030-081).

LUCIFERASE ASSAYS: Our transient transfection luciferase assay was described previously by us (Stockwell et al., Chem Biol 5:385, 1998). Briefly, 100,000 Mv1Lu mink lung epithelial cells were transiently transfected in 12-well dishes with 400 ng p3TPLux or pNFkB-Lux, with or without 50 ng pFC-MEKK, in 300 μL minimal essential medium with non-essential amino acids. The DEAE-dextran/chloroquine/DMSO methods was used for transfection (Stockwell et al., Chem Biol 6:71, 1999). After cell lysis in 120 μL lysis buffer, a Beckman LS 6500 liquid scintillation counter was used in single photon mode to quantitate luminescence. For detection of luciferase activity in 6F cells (including the primary screen), 20,000 6F cells were seeded in 50 μL of 10% mink medium in each well of a white 384-well plate (Nalge Nune International; Naperville, Ill.; cat#164610) using a Multidrop 384 liquid dispenser (Lab Systems; Helsinki, Finland). After 16 hours, medium was removed using a 24 channel wand (V&P Scientific, Inc.; San Diego, Calif.; cat#VP 186L), the cells were washed with 75 μL of 0.2% mink medium (containing 0.2% FBS), and reagents were added in 40 μL of 0.2% medium. For the primary screen, reagents were added by pin transfer using 384 polypropylene pin arrays (Matrix Technologies; Hudson, N.H.). After 24 hours, the cells were cooled on ice and washed twice with 75 μL Hanks Balanced Salt Solution (HBSS; GibcoBRL; cat#24020-117). Then 20 μL lysis buffer (25 mM glycylglycine (Sigma; cat#E-0396), 1% Triton X-100 (Sigma; cat#T-9284), 1 mM dithiothreitol (DTT; Sigma; cat#D-5545), 1 mM phenylmethylsulfonyl fluoride (PMSF; Sigma; cat#P-7626)) was added to each well with a Multidrop. After incubating the cells for five minutes on ice, 20 μL of ATP/luciferin solution was added (25 mM glycylglycine pH 7.8, 15 mM MgSO₄, 4 mM EGTA, 6.25 mM K₂HPO₄ (Sigma; cat#P5504) pH 7.8, 5 mM DTT, 75 μM D-luciferin (Sigma, cat# L-9504, 2 mM ATP (Sigma; cat#A-7699)). Light output was immediately measured with an Analyst 384-well platereader (LJL), with 0.5 second counting time per well.

BRDU CYTOBLOT ASSAY: The BrdU cytoblot assay for S-phase progression was described previously by Stockwell et al. (Chem. Biol. 6:71, 1999; see also, U.S. Patent Application Ser. No. 60/094,305, incorporated herein by reference).

TRANSCRIPTIONAL PROFILING: We performed transcriptional profiling on yeast cells according to known techniques. In a control transcriptional profiling experiment, we compared two different cultures of untreated yeast of the same strain and found variations of 0.74-2.0 fold in expression of particular genes. We therefore set thresholds of 0.5 fold and 2.0 fold for transcriptional repression and transcriptional activation, respectively, in our experimental comparisons of untreated cells and cells treated with test compounds.

UV SPECTRAL SHIFT BINDING ASSAY: A quartz cuvette with 1 mL deionized, distilled water was used as a blank on a Cary 1E UV-visible spectrophotomer. 1a or 2 was diluted to a final concentration of 15 μM or 20 μM, respectively, and the UV spectrum from 240 nm to 320 nm was measured. In the absence of metals, a shoulder in UV spectrum of 1a and 2 had a λₘₐₓ of 292 and 297 nm, respectively. Upon addition of certain transition metals (Table 1), this peak shifted such that the new λₘₐₓ was 273 nm and 310 nm, for 1a and 2, respectively. Metal salts were titrated into the solution, starting from 10 nM and going up to 1 mM, or the limit of the solubility of the metal, with 2-3 fold increments in concentration. The concentration of metal ion at which the UV spectrum had shifted approximately 50% was reported as “EC₅₀ (UV)” in Table 1. For the calculation of correlation coefficients, all values less than 7.5 μM were taken to be 7.5 μM, all values greater than 1000 μM were taken to be 1000 μm and all values less than 10 μM were taken to be 10 μM.

TRANSCRIPTIONAL PROFILING In S. CEREVISIAE: The protocol of James Hardwick and Jeffrey Tong was used (James Hardwick, Jeffrey Tong, and Stuart L. Schreiber, unpublished results). Briefly, S. cerevisiae strain BY4741 (haploid) was streaked on a YPD plate, grown for 3 days at 30° C., and used to inoculate a 50 mL overnight culture of YPD. This culture was diluted into pre-warmed YPD such that A₆₀₀=0.04 and allowed to grow until A₆₀₀=0.47. The culture was split into pre-warmed flasks, with 50 mL in each of three flasks and 150 mL in a fourth flask. The 150 mL culture and one 50 mL culture were not treated with any reagent and after 4 hours grew to A₆₀₀=1.2. One 50 mL culture was treated with 200 μM 2 and grew to A₆₀₀=0.56. The cultures were centrifuged at 2500 g at room temperature for five minutes and the pelle frozen in liquid nitrogen. Total RNA was purified from each culture by hot acidic phenol/chloroform extraction and ethanol precipitation. Poly-A RNA wad purified with a Qiagen Oligotex mRNA Midi Kit (Qiagen; Valencia, Calif.; cat#70042). Fluorescently-labeled probe was prepared from 1.25 pg poly-A mRNA and applied to glass slides that had been printed with 6240 yeast ORFs (James Hardwick, Jeffrey Tong, and Stuart L. Schreiber, unpublished results). The microarray was scanned with an Array Works scanner and the results were analyzed with Gene Spring software.

### Results/Discussion

We screened more than 16,000 chemical compounds to identify those that specifically activated expression of a reporter gene (luciferase) under the control of TGFβ-responsive elements in stably transfected mammalian cells (mink lung epithelial cells). We identified four compounds, 1a-c and 2 (see FIG. 13), that showed activity in our screen. Given the structural similarity of compounds 1a-c, we expected that all three were operating through a common mechanism. We therefore continued testing on only one, compound 1a, as representative of the group.

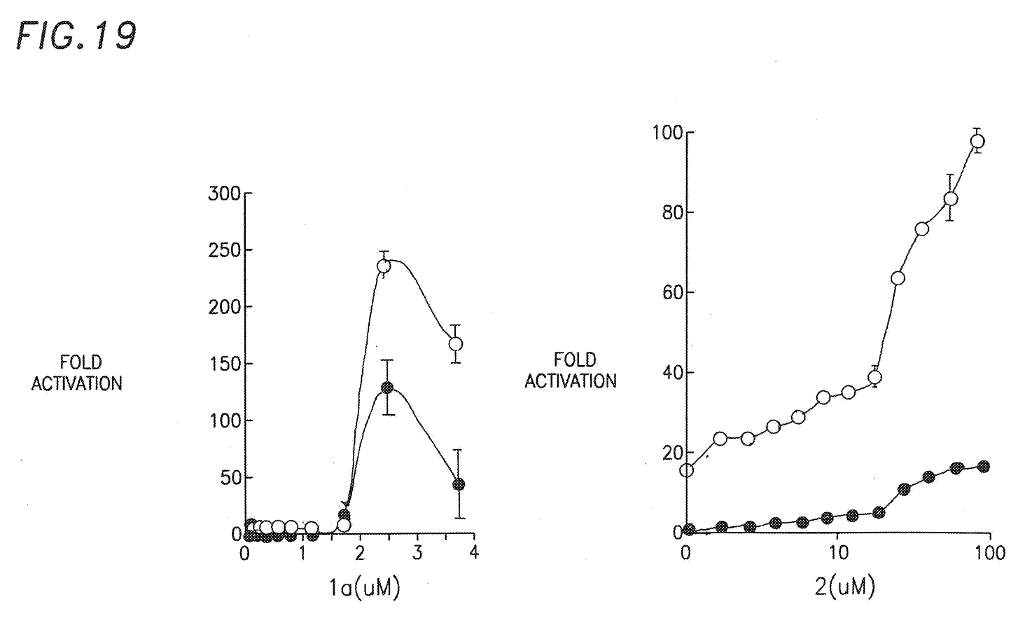

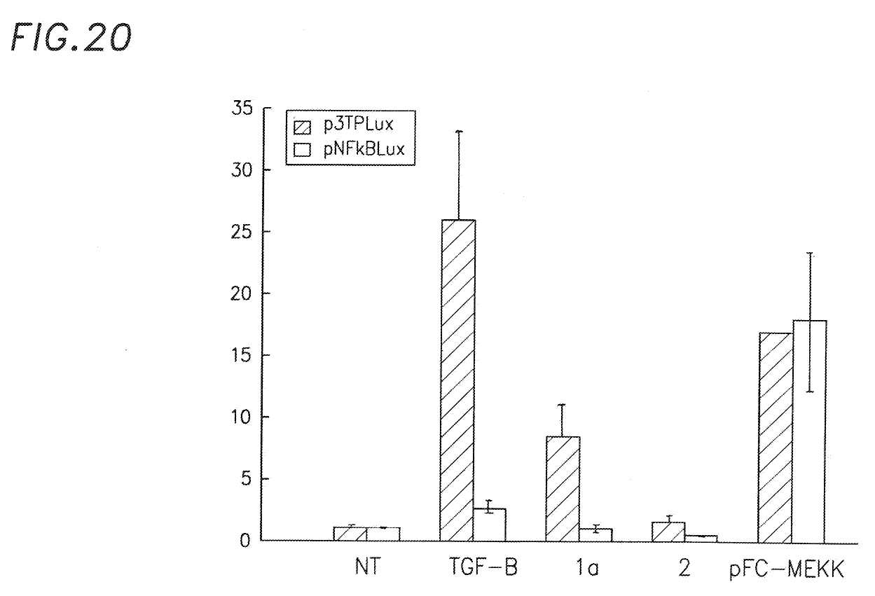

Compounds 1a and 2 each induce a dose-dependent increase in reporter gene expression; each is also synergistic with TGFβ (see FIG. 19). Neither compound activated a transiently transfected control reporter responsive to NFκB (see FIG. 20). Interestingly, each compound was less effective at activating a transiently transfected TGFβ-responsive reporter as compared with a stably transfected reporter (compare transient transfection results in FIG. 20 with stable transfection results in FIG. 18). TGFβ was equally effective at activating both types of reporters.

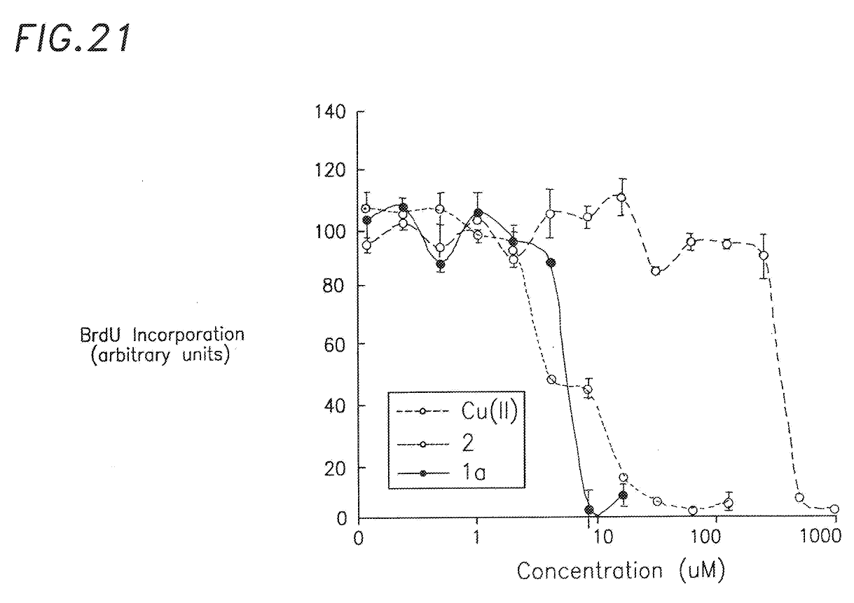

Compounds that mimic TGFβ activity would be expected to be capable of inducing cell cycle arrest or apoptosis, and in particular to block DNA replication, in responding cells. We therefore used an S-phase progression cytoblot assay to test whether our compounds could inhibit 5-bromodeoxyuridine (BrdU) incorporation into mink epithelial cells. As shown in FIG. 21, compounds 1a and 2 both inhibited BrdU incorporation. Both compounds were therefore classified as true TGFβ mimics.

We performed transcriptional profiling experiments in yeast to determine whether our compounds activated or repressed transcription of genes within that organism. Yeast do not have a TGFβ pathway per se but we nonetheless expected the transcriptional profiling might yield information about the mechanism by which our compounds alter gene expression. Compound 2 had no detectable effect on gene expression in yeast cells, perhaps indicating that the compound is not taken up into those cells. Table 1, below, summarizes the result of our transcriptional profiling experiment with compound 1a:

TABLE 1
Transcriptional Profiling of Compound 1a in Yeast Cells

| | | |
|---|---|---|
| | | EXPRESSION LEVEL |
| | | IN PRESENCE OF |
| | | COMPOUND |
| | | EXPRESSION LEVEL |
| | | IN ABSENCE OF |
| GENE | ENCODED PROTEIN | COMPOUND |

REPRESSED GENES

| | | |
|---|---|---|
| YBR150C | Zn-containing transcription | 0.48 |
| | factor | |
| YAL045C | Multi-drug resistance pump | 0.49 |
| (ARN1) | | |

ACTIVATED GENES

| | | |
|---|---|---|
| YBR072W | Heat shock protein | 2.8 |
| (HSP26) | | |
| YMR058W | Multicopper oxidase | 2.4 |
| (FET3) | | |
| YGL255W | High affinity Zn transporter | 2.3 |
| (ZRT1) | | |
| YBL005WA | TyA transposon | 2.2 |
| YDR037W | lysyl tRNA synthetase | 2.2 |
| YMR051C | TyA gag protein | 2.2 |
| YBL101WA | TyA gag protein | 2.1 |
| YBR054W | similar to HSP30 | 2.1 |
| YBR207W | similar to iron transporter | 2.1 |
| YDR534C | unknown | 2.1 |
| YBL092W | ribosomal protein | 2.1 |
| (RP32) | | |
| YDR023W | serine tRNA synthase | 2.0 |
| (SES1) | | |
| YCL064C | ser/thr deaminase | 2.0 |
| YHL040C | multidrug resistance pump | 2.0 |
| (ARN1) | | |

We expect that induction of the ARN1 multidrug resistance pump and heat shock protein genes probably reflect the stress imposed on cells by the experiment rather than any specific activity of compound 1a. Several of the other induced genes encode transition metal transporters. Without wishing to be bound by any particular theory, we propose that compound 1a binds to transition metals, and that yeast exposed to the compound respond by increasing the expression of transition metal transporters. In particular, our results suggested that compound 1a binds to copper, iron, and/or zinc in yeast cells. We therefore tested whether these metals affected the ability of compound 1a (or compound 2) to activate transcription in mammalian cells.

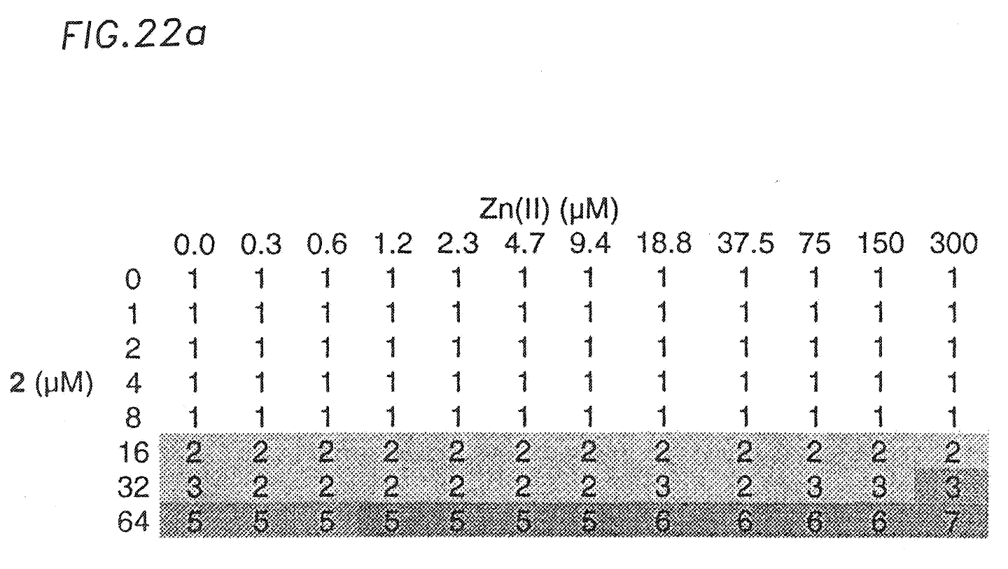

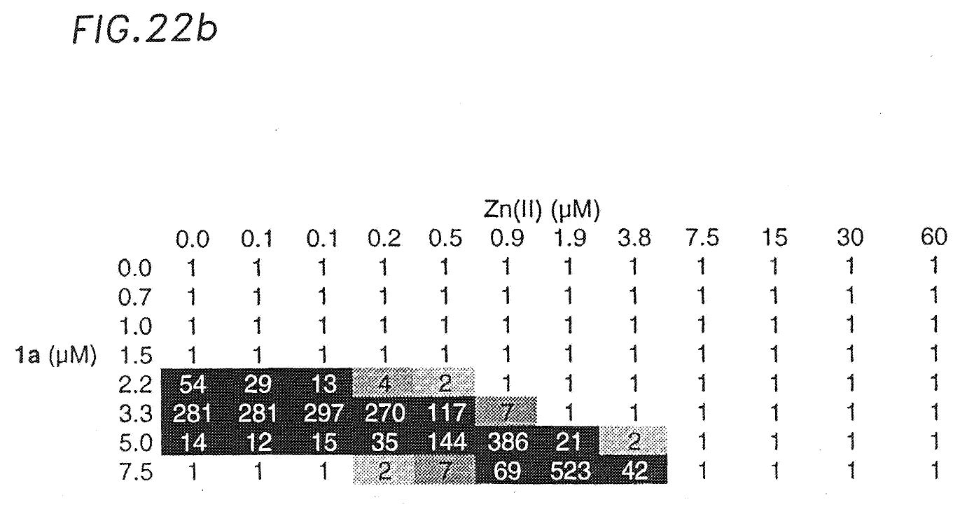

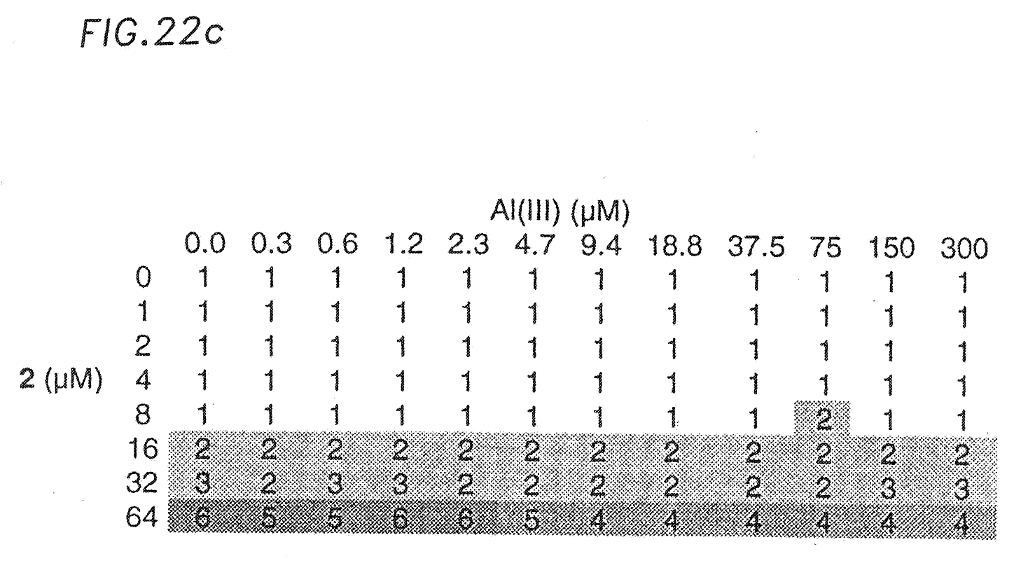

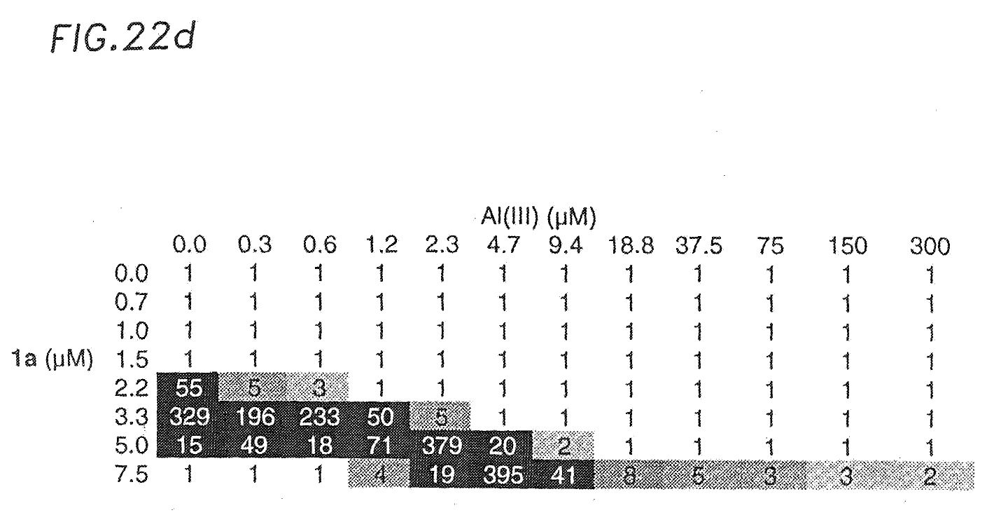

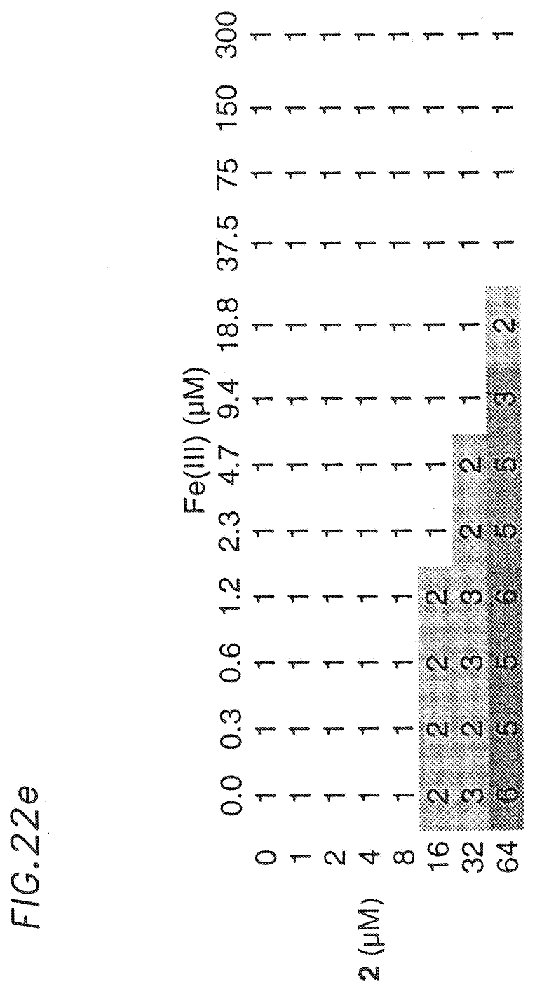

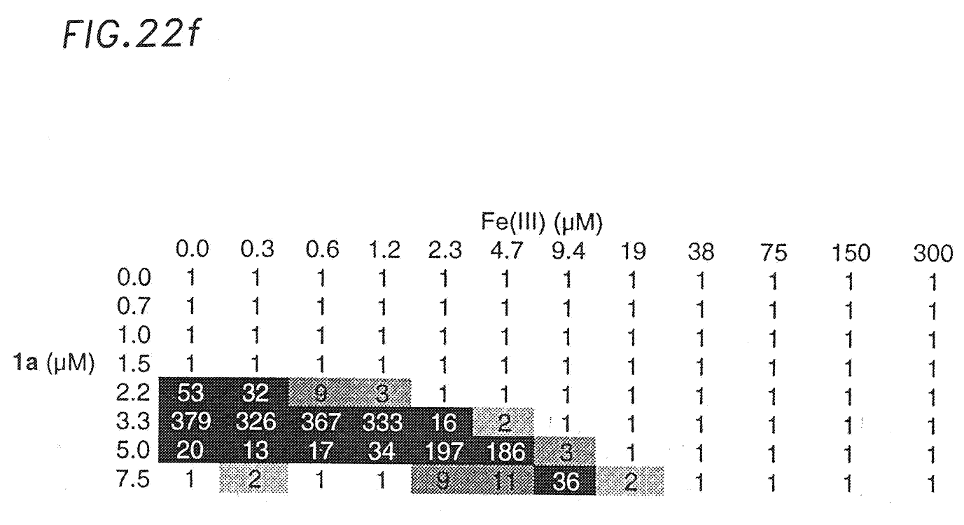

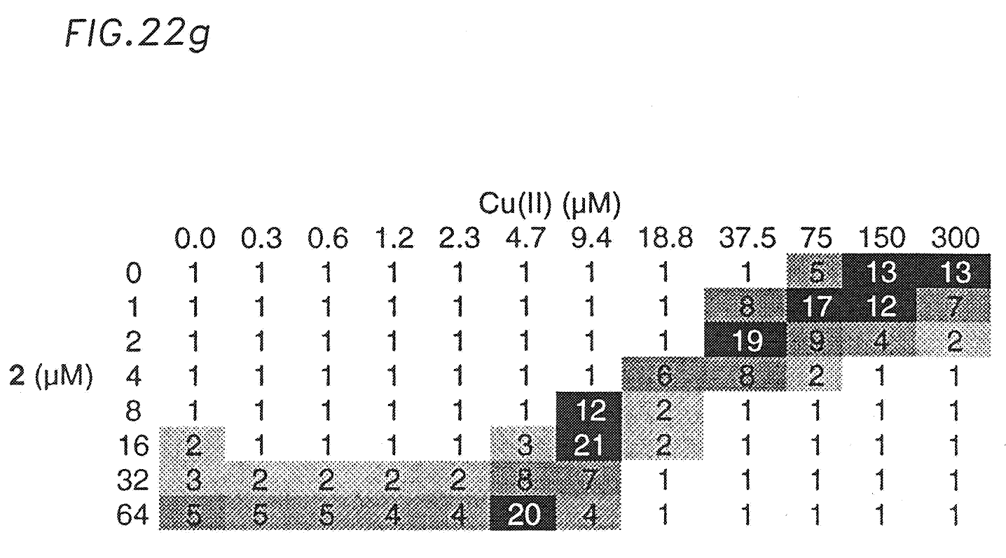

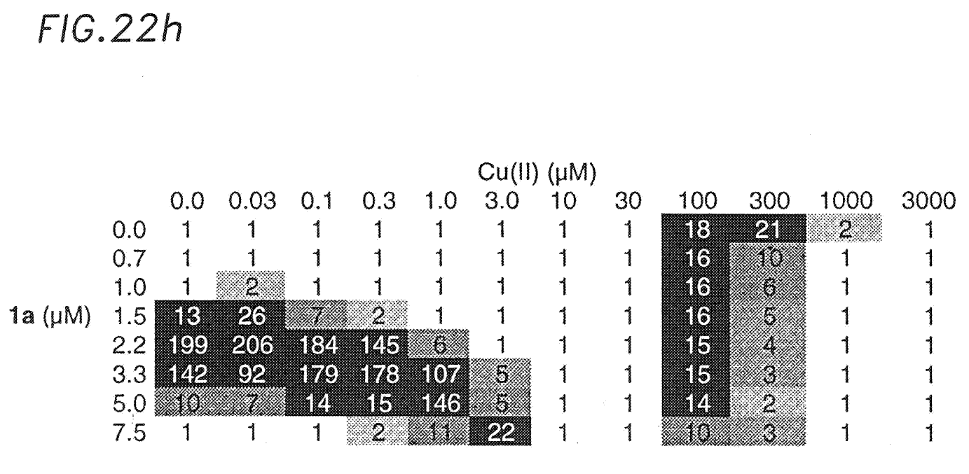

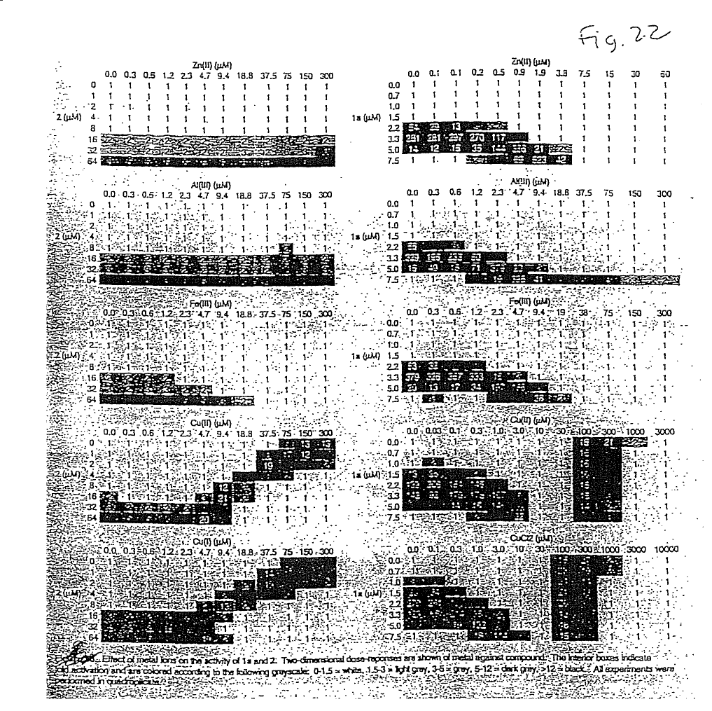

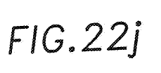

As shown in Table 2 and FIG. 22, Fe³⁺ and Co²⁺ completely suppressed the ability of 1a and 2 to activate expression of our TGFβ-responsive reporter in mink lung epithelial cells; these metals had no effect on TGFβ's ability to activate. Zn²⁺, Mn²⁺, Al³⁺ and Ni²⁺ also suppressed transcriptional activation by 1a, but had no effect on activation by 2 or TGFβ. Alkali and alkali earth metals did not affect reporter gene activation by 1a, 2, or TGFβ.

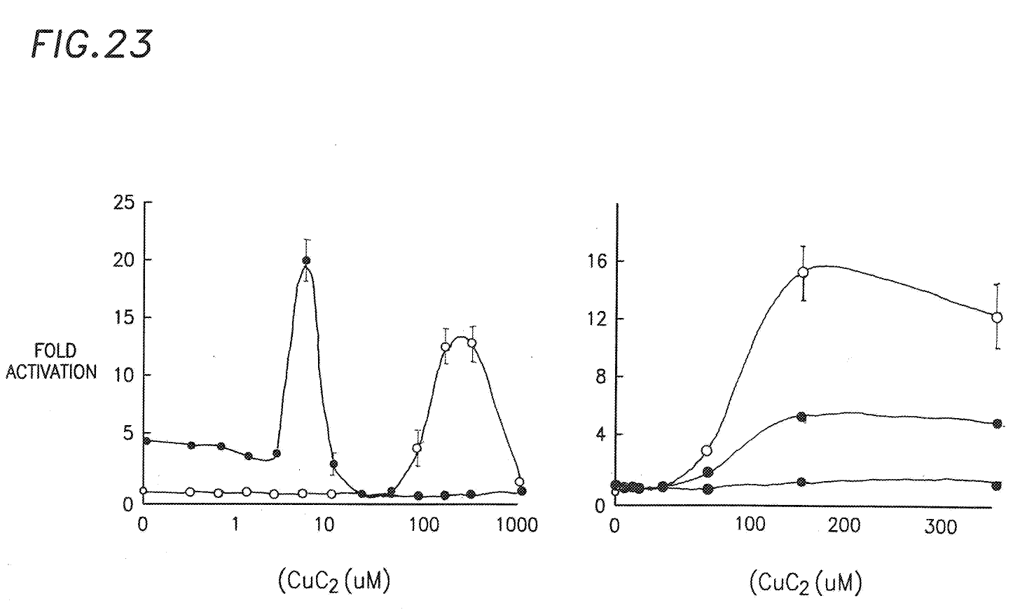

Interestingly, Cu⁺ and Cu²⁺ each had the ability to activate the reporter gene alone (see FIG. 23). Moreover, 2 synergized strongly with both Cu⁺ and Cu²⁺ but not with other metals (see FIG. 22). Transcriptional stimulation by 2 was blocked by a known copper chelator (diethyldithiocarbamate [DDC]), which also suppressed activation by copper and by TGFβ but not by 1a. In light of these results, we hypothesized that 2 acts as a copper transporter and that elevated levels of copper activate the TGFβ reporter. Fe³⁺ and Co²⁺ may inhibit 2's activity by competing with copper for binding to 2.

We tested the ability of 1a and 2 to bind directly to a number of different metals by measuring each compound's absorbance of ultraviolet (UV) light from 240-310 nm in the presence of various metals. A shift in absorbance maximum (λₘₐₓ) in the presence of the metal indicates an ability to bind to the metal. As shown in Table 2, 1a binds strongly to Zn²⁺, Fe²⁺, Cu²⁺, Cu⁺, and Al³⁺, and less well to Mn²⁺, Co²⁺, and Ni²⁺; 2 binds well to Fe³⁺, Cu²⁺, Cu⁺, and CO²⁺, and also shows some binding to Ni²⁺. Interestingly, the affinity of inhibitory metals for the compound whose activity they inhibit correlates well with the extent of inhibition (0.99 for 1a and 0.97 for 2). Also, Zn²⁺ suppressed the ability of copper to activate reporter gene expression, but Al³⁺, Ni²⁺, Co²⁺, Fe²⁺, and Mn²⁺ did not.

Without wishing to be bound by any particular theory, we propose that our reporter gene is activated by high concentrations of copper and low concentrations of zinc. We further propose that 2 stimulates expression of this reporter by acting as a copper transporter and increasing the local concentration of copper; 1a may act as a zinc chelator that decreases the local concentration of zinc. One possible explanation for the observed increase in reporter gene expression in the presence of copper and the absence of zinc would be the existence of an inhibitory protein whose activity requires zinc. High concentrations of copper may cause the copper to exchange for zinc in the protein, thereby inactivating the protein and derepressing the gene. Given that 1a and 2 both activate stably transfected reporters more effectively than transiently transfected reporters, it may be that the zinc-dependent inhibitor of gene expression is chromatin-remodeling agent. One possible candidate inhibitor TGIF, a recently-described repressor of the p3TPLux reporter (Wotton et al., Cell 97:29, 1999). Interestingly, the closest yeast homolog of TGIF is cup9, which was isolated in a screen for copper-resistant genes (Knight et al., Mol. Cell. Biol. 14:7792, 1994).

In order to probe the mechanism by which our identified compounds stimulate expression of our TGFβ-responsive reporter, we asked whether agents that affect free radical formation (i.e., H2O2 or AIBN) mimicked or suppressed their activity. We found that neither agent altered the effect of 1a, 2, or copper on our reporter construct. Also, the presence of a neutralizing TGFβ antibody did not alter the activity of 1a, 2, or copper in our system, indicating that none of these compounds acts by up-regulating TGFβ itself.

These studies have identified compounds that activate TGFβ-responsive genes and inhibit cell proliferation by some mechanism other than altering free radical formation or increasing TGFβ concentration. These compounds also bind to metals, apparently as metal transporters or metal chelators. We performed additional studies to investigate the structural elements responsible for these activities.

As mentioned above, three of the four compounds we identified (i.e., compounds 1a, 1b, and 1c) are bis(8-quinolinol)s, with methyl, hydrogen, and n-butyl R groups, respectively. These three compounds were the only soluble bis(8-quinolinol)s in the collection of compounds that we screened. It is almost certain that other bis(8-quinolinol)s would have the same activities. Certainly, short chain (e.g., fewer than about 10 carbons and preferably fewer than about 5 carbons) would likely behave in the same way.

The collection of compounds that we tested included approximately 15 compounds similar in structure to monomeric 8-hydroxyquinoline, but none of these activated transcription of our TGFβ-responsive reporter. Also, we separately tested 8-hydroxyquinoline itself and found that it did not activate our reporter. The dimeric structure of compounds 1a-c is therefore probably important for their activity.

TABLE 2
Metal Binding by 1a and 2 Correlates
with Sensitivity of Metal Antagonism

| | EC₅₀UV | EC₅₀SU | EC₅₀UV | EC₅₀Su |
|---|---|---|---|---|
| Metal^{a} | 1a (μM)^{b} | 1a (μM)^{c} | 2 (μM)^{d} | 2 (μM)^{c} |
| Zn²⁺ | <7.5 | 0.5 | 500 | >300 |
| Fe³⁺ | <7.5 | 2.0 | <10 | 9 |
| Cu²⁺ | 10 | 1.0 | <10 | * |
| Cu⁺ | 15 | 2.0 | <10 | * |
| Al³⁺ | 30 | 1.0 | 200 | >300 |
| Mn²⁺ | 80 | 200 | >1000 | >300 |
| Co²⁺ | 100 | 10 | 50 | 30 |
| Ni²⁺ | 280 | 10 | 500 | 200 |
| Mg²⁺ | >1000 | >1000 | >1000 | >1000 |
| K⁺ | >1000 | >1000 | >1000 | >1000 |
| Ca²⁺ | >1000 | >1000 | >1000 | >1000 |
| Na⁺ | >1000 | >1000 | >1000 | >1000 |
| Ba²⁺ | >1000 | >1000 | >1000 | >1000 |

^{a}All metals were used in the form of the chloride salts, except for NiSO₄ and Ba (OAc)₂.
^{b}Concentration at which the metal ion induces a 50% shift in the λₘₐₓ of a shoulder in the UV spectrum of 1a ([1a] = 15 μM) from 292 nm to 273 nm. Assuming a 1:1 stoichiometric complex, this EC₅₀ is an estimate of the dissociation constant for the complex.
^{c}Concentration at which metal ion inhibits 50% of the activation of p3TPLux by 1a or 2 in 6F mink lung cells ([1a] = 3 μM, [2] = 64 μM).
^{d}Concentration at which the metal ion induces a 50% shift in the λₘₐₓ of a shoulder in the UV spectrum of 2 from 297 nm to 310 nm ([2] = 20 μM).

What is claimed is:

1-56. (canceled)

57. A high-throughput method for screening one or more test compounds to identify those that exert an effect on post-translational modification of a polypeptide, the method comprising steps of:
a. introducing into each of a plurality of reaction vessels:
a plurality of cells; and
one or more test compounds whose effect on post-translational modification of a polypeptide is to be evaluated;
b. introducing into at least some of the reaction vessels an antibody characterized in that it associates intracellularly with a biological component whose presence or amount reveals the effect of a given test compound on post-translational modification of the polypeptide, the introducing being performed under conditions and for a time sufficient that the antibody enters one or more cells and associates intracellularly with the biological component; and
c. assaying for association between the antibody and the biological component in the reaction vessels to assess the presence or amount of the biological component, thereby revealing the effect of the test compound on the post-translational modification of the polypeptide;
wherein the plurality of reaction vessels comprises at least 96 reaction vessels.

58. A high-throughput method for obtaining a functional fingerprint of one or more test compounds; said method comprising steps of:
a. introducing into each of a plurality of reaction vessels:
a plurality of cells; and
one or more test compounds whose effects on a plurality of post-translational modification events of polypeptides are to be recorded as a functional fingerprint;
b. introducing into at least some of the reaction vessels an antibody characterized in that it associates intracellularly with a biological component whose presence or amount reveals the effect of a given test compound on a given polypeptide post-translational modification event; the introducing being performed under conditions and for a time sufficient that the antibody enters one or more cells and associates intracellularly with the biological component; wherein a plurality of antibodies are introduced into distinct reaction vessels for detection of a plurality of post-translational modification events;
c. assaying for association between the antibody and the biological component in each reaction vessel to assess the presence or amount of the biological component, thereby revealing the effect of the test compound on the given polypeptide post-translational modification event; and
d. recording the effects of each test compound on the plurality of post-translational modification events of polypeptides, thereby establishing a functional fingerprint for each test compound;
wherein the plurality of reaction vessels comprises at least 96 reaction vessels.

59. The method of claim 57 or 58 further comprising the step of removing unassociated antibody from each reaction vessel.

60. (canceled)

61. (canceled)

62. (canceled)

63. The method of claim 57 or 58 wherein the antibody is conjugated to horseradish peroxidase.

64. The method of claim 57 or 58 wherein the method further comprises introducing a secondary ligand that binds specifically to said antibody, and wherein the step of assaying comprises assaying for bound secondary ligand.

65. (canceled)

66. The method of claim 64 wherein in the step of assaying, the secondary ligand is assayed intracellularly.

67. The method of claim 64 wherein the secondary ligand is an antibody.

68. The method of claim 67 wherein the antibody is conjugated to horseradish peroxidase.

69. The method of claim 57 or 58 wherein the step of assaying utilizes a detection technique selected from the group consisting of: chemiluminescence, fluorescence, phosphorescence, radioactivity, colorimetry, Ultra-Violet spectroscopy, and Infra-Red spectroscopy.

70. (canceled)

71. The method of claim 57 or 58 wherein, in the step of introducing the cells in each of the plurality of reaction vessels, the cells adhere to the reaction vessel surface.

72. (canceled)

73. (canceled)

74. (canceled)

75. (canceled)

76. The method of claim 57 or 58 wherein the post-translational modification is a covalent modification of an intracellular polypeptide.

77. The method of claim 76 wherein the covalent modification is an intracellular biological reaction.

78. (canceled)

79. (canceled)

80. The method of claim 76 wherein the post-translational modification is glycosylation, methylation, lipidation, isoprenylation, ubiquitination, phosphorylation or acetylation.

81. (canceled)

82. (canceled)

83. The method of claim 57 or 58 wherein the cells are from the same cell-line.

84. (canceled)

85. The method of claim 57 or 58 wherein at least a subset of the cells comprises a eukaryotic cell.

86. The method of claim 57 or 58 wherein at least a subset of the cells comprises a mammalian cell.

87. The method of claim 57 or 58 wherein at least a subset of the cells comprises a human cell.

88. The method of claim 57 or 58 wherein at least one test compound is from a synthetic source.

89. The method of claim 88 wherein the test compounds are from a combinatorial library.

90. The method of claim 89 wherein the test compounds are covalently bound on a solid support, the method further comprising the step of dissociating the test compounds from the solid support.

91. The method of claim 57 or 58 wherein the reaction vessels are designed to receive a volume of liquid less or equal to approximately 200 microliters.

92. The method of claim 57 or 58 wherein the reaction vessels are designed to receive a volume of liquid less or equal to approximately 50 microliters.

93. The method of claim 57 or 58 wherein the reaction vessels are designed to receive a volume of liquid less or equal to approximately 2 microliters.

94. The method of claim 57 or 58 wherein the reaction vessels are designed to receive a volume of liquid less or equal to approximately 250 nanoliters.

95. The method of claim 57 or 58 wherein the reaction vessels are arranged in a two-dimensional array with sufficient density that the center-to-center distance between adjacent vessels is less than about 8.5 millimeters.

96. The method of claim 57 or 58 wherein the reaction vessels are arranged in a two-dimensional array with sufficient density that the center-to-center distance between adjacent vessels is less than about 4.5 millimeters.

97. The method of claim 57 or 58 wherein the reaction vessels are arranged in a two-dimensional array with sufficient density that the center-to-center distance between adjacent vessels is less than about 2.25 millimeters.

98. The method of claim 57 or 58 wherein the reaction vessels are arranged in a two-dimensional array with sufficient density that the center-to-center distance between adjacent vessels is less than about 1 millimeter.

99. The method of claim 57 or 58 wherein the number of reaction vessels is greater than or equal to approximately 384 and the reaction vessels occupy a surface smaller than or equal to approximately 128×86 mm².

100. The method of claim 57 or 58 wherein the number of reaction vessels is greater than or equal to approximately 1500 and the reaction vessels occupy a surface smaller than or equal to approximately 128×86 mm².

101. The method of claim 57 or 58 wherein the number of reaction vessels is greater than or equal to approximately 6000 and the reaction vessels occupy a surface smaller than or equal to approximately 128×86 mm².

102. The method of claim 57 or 58 wherein in the step of introducing the test compounds into the plurality of reaction vessels, the test compounds are the same or different.

103. The method of claim 57 or 58 wherein in the step of introducing the test compounds into the plurality of reaction vessels, each reaction vessel contains one test compound.

104. The method of claim 57 or 58 wherein at least one test compound is from a natural source.

105. The method of claim 58 wherein the reaction vessels are wells of a 96-, 384-, 1536- or 6144-well plate.

106. The method of claim 105 wherein the same test compound is introduced in each of the wells and a different antibody is introduced in each well.

107. The method of claim 105 wherein a the same test compound and a different antibody are introduced in each well across a row, and a different test compound and the same antibody are introduced in each well down a column.

108. The method of claim 57, wherein the antibody associates intracellularly with the polypeptide after post-translational modification.

109. The method of claim 57, wherein the antibody associates intracellularly with the polypeptide prior to posttranslational modification.

110. The method of claim 57 or 58 wherein the step of assaying utilizes chemiluminescence.
